SEQUENCE LISTING (167 sequences)

SEQ ID NO: 1 - protein, 235 aa, Saccharomyces cerevisiae
MKNIKKNQVMNLGPNSKLLKEYKSQLIELNIEQFEAGIGLILGDAYIRSRDEGKTYCMQFEWKNKAYMDHVCLLYDQWVLSPPHKKERVNHLGNLVITWGAQTFKHQAFNKLANLFIVNNKKTIPNNLVENYLTPMSLAYWFMDDGGKWDYNKNSTNKSIVLNTQSFTFEEVEYLVKGLRNKFQLNCYVKINKNKPIIYIDSMSYLIFYNLIKPYLIPQMMYKLPNTISSETFLK

SEQ ID NO: 2 - protein, 236 aa, Artificial Sequence
  derived from I-SceI sequence
MGKNIKKNQVMNLGPNSKLLKEYKSQLIELNIEQFEAGIGLILGDAYIRSRDEGKTYCMQFEWKNKAYMDHVCLLYDQWVLSPPHKKERVNHLGNLVITWGAQTFKHQAFNKLANLFIVNNKKTIPNNLVENYLTPMSLAYWFMDDGGKWDYNKNSTNKSIVLNTQSFTFEEVEYLVKGLRNKFQLNCYVKINKNKPIIYIDSMSYLIFYNLIKPYLIPQMMYKLPNTISSETFLK

SEQ ID NO: 3 - protein, 227 aa, Artificial Sequence
  derived from I-SceI sequence
MGKNIKKNQVMNLGPNSKLLKEYKSQLIELNIEQFEAGIGLILGDAYIRSRDEGKTYCMQFEWKNKAYMDHVCLLYDQWVLSPPHKKERVNHLGNLVITWGAQTFKHQAFNKLANLFIVNNKKTIPNNLVENYLTPMSLAYWFMDDGGKWDYNKNSTNKSIVLNTQSFTFEEVEYLVKGLRNKFQLNCYVKINKNKPIIYIDSMSYLIFYNLIKPYLIPQMMYKLPN

SEQ ID NO: 4 - protein, 7 aa, SV40
PKKKRKV

SEQ ID NO: 5 - protein, 234 aa, Artificial Sequence
  derived from I-SceI sequence ; S. cerevisiae
MGPKKKRKVKNIKKNQVMNLGPNSKLLKEYKSQLIELNIEQFEAGIGLILGDAYIRSRDEGKTYCMQFEWKNKAYMDHVCLLYDQWVLSPPHKKERVNHLGNLVITWGAQTFKHQAFNKLANLFIVNNKKTIPNNLVENYLTPMSLAYWFMDDGGKWDYNKNSTNKSIVLNTQSFTFEEVEYLVKGLRNKFQLNCYVKINKNKPIIYIDSMSYLIFYNLIKPYLIPQMMYKLPN

SEQ ID NO: 6 - protein, 439 aa, Escherichia coli Tn10
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGSSGGGGSGGGGSGGGGSGGGGSGGGGMSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGS

SEQ ID NO: 7 - protein, 121 aa, Artificial Sequence
  P22, protein similar to the scArcR protein
MGGMSKMPQFNLRWPREVLDLVRKVAEENGRSVNSEIYQRVMESFKKEGRIGAGGGSGGGTGGGSGGGMKGMSKMPQFNLRWPREVLDLVRKVAEENGRSVNSEIYQRVMESFKKEGRIGA

SEQ ID NO: 8 - protein, 675 aa, Artificial Sequence
  chimeric protein
MGKNIKKNQVMNLGPNSKLLKEYKSQLIELNIEQFEAGIGLILGDAYIRSRDEGKTYCMQFEWKNKAYMDHVCLLYDQWVLSPPHKKERVNHLGNLVITWGAQTFKHQAFNKLANLFIVNNKKTIPNNLVENYLTPMSLAYWFMDDGGKWDYNKNSTNKSIVLNTQSFTFEEVEYLVKGLRNKFQLNCYVKINKNKPIIYIDSMSYLIFYNLIKPYLIPQMMYKLPNTISSETFLKLSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGSSGGGGSGGGGSGGGGSGGGGSGGGGMSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGS

SEQ ID NO: 9 - protein, 682 aa, Artificial Sequence
  chimeric protein
MGPKKKRKVKNIKKNQVMNLGPNSKLLKEYKSQLIELNIEQFEAGIGLILGDAYIRSRDEGKTYCMQFEWKNKAYMDHVCLLYDQWVLSPPHKKERVNHLGNLVITWGAQTFKHQAFNKLANLFIVNNKKTIPNNLVENYLTPMSLAYWFMDDGGKWDYNKNSTNKSIVLNTQSFTFEEVEYLVKGLRNKFQLNCYVKINKNKPIIYIDSMSYLIFYNLIKPYLIPQMMYKLPNTISSETFLKLSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGSSGGGGSGGGGSGGGGSGGGGSGGGGMSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGS

SEQ ID NO: 10 - protein, 383 aa, Artificial Sequence
  chimeric protein
MGGMSKMPQFNLRWPREVLDLVRKVAEENGRSVNSEIYQRVMESFKKEGRIGAGGGSGGGTGGGSGGGMKGMSKMPQFNLRWPREVLDLVRKVAEENGRSVNSEIYQRVMESFKKEGRIGARSGGGSGGGTGGGSGGGAPKKKRKVLEMKNIKKNQVMNLGPNSKLLKEYKSQLIELNIEQFEAGIGLILGDAYIRSRDEGKTYCMQFEWKNKAYMDHVCLLYDQWVLSPPHKKERVNHLGNLVITWGAQTFKHQAFNKLANLFIVNNKKTIPNNLVENYLTPMSLAYWFMDDGGKWDYNKNSTNKSIVLNTQSFTFEEVEYLVKGLRNKFQLNCYVKINKNKPIIYIDSMSYLIFYNLIKPYLIPQMMYKLPNTISSETFLK

SEQ ID NO: 11 - protein, 368 aa, Artificial Sequence
  chimeric protein
MGGMSKMPQFNLRWPREVLDLVRKVAEENGRSVNSEIYQRVMESFKKEGRIGAGGGSGGGTGGGSGGGMKGMSKMPQFNLRWPREVLDLVRKVAEENGRSVNSEIYQRVMESFKKEGRIGARSAPKKKRKVLEMKNIKKNQVMNLGPNSKLLKEYKSQLIELNIEQFEAGIGLILGDAYIRSRDEGKTYCMQFEWKNKAYMDHVCLLYDQWVLSPPHKKERVNHLGNLVITWGAQTFKHQAFNKLANLFIVNNKKTIPNNLVENYLTPMSLAYWFMDDGGKWDYNKNSTNKSIVLNTQSFTFEEVEYLVKGLRNKFQLNCYVKINKNKPIIYIDSMSYLIFYNLIKPYLIPQMMYKLPNTISSETFLK

SEQ ID NO: 12 - protein, 30 aa, Artificial Sequence
  linker peptide
IGARSGGGSGGGTGGGSGGGAPKKKRKVLE

SEQ ID NO: 13 - DNA, 18 bp, Artificial Sequence
  chimeric recognition site
tagggataacagggtaat

SEQ ID NO: 14 - DNA, 34 bp, Artificial Sequence
  chimeric recognition site
ctatcaatgatagcgctagggataacagggtaat

SEQ ID NO: 15 - DNA, 35 bp, Artificial Sequence
  chimeric recognition site
ctatcaatgatagacgctagggataacagggtaat

SEQ ID NO: 16 - DNA, 36 bp, Artificial Sequence
  chimeric recognition site
ctatcaatgatagtacgctagggataacagggtaat

SEQ ID NO: 17 - DNA, 31 bp, Artificial Sequence
  chimeric recognition site
tagggataacagggtaatactagtagagtgc

SEQ ID NO: 18 - DNA, 32 bp, Artificial Sequence
  chimeric recognition site
tagggataacagggtaatacttagtagagtgc

SEQ ID NO: 19 - DNA, 33 bp, Artificial Sequence
  chimeric recognition site
tagggataacagggtaatactatagtagagtgc

SEQ ID NO: 20 - DNA, 34 bp, Artificial Sequence
  chimeric recognition site
tagggataacagggtaatactagtagtagagtgc

SEQ ID NO: 21 - DNA, 4065 bp, Artificial Sequence
  plasmid
ccnnnnnnnnnngaattcgaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggctaacaggaggaattaa

SEQ ID NO: 22 - DNA, 711 bp, Artificial Sequence
  Insert of VC-SAH40-4
atgggtaagaacattaagaagaaccaggtgatgaacctgggccctaactctaagctgcttaaggaatacaagtctcagctgattgagctgaacattgagcagttcgaggctggcataggcctgattctgggcgatgcttacattaggtctagggatgagggcaagacctactgcatgcagttcgagtggaagaacaaggcttacatggatcacgtgtgcctgctgtacgatcagtgggtgctgtctcctcctcacaagaaggagagggtgaaccacttgggaaacctggtgattacctggggcgctcaaaccttcaagcaccaggctttcaacaagctggctaacctgttcattgtgaacaacaagaagaccattcctaacaacctggtggagaactacctgacccctatgtctctggcttactggttcatggatgatggcggcaagtgggattacaacaagaactctaccaacaagtctattgtgctgaacacccagtctttcaccttcgaggaggtggaatacctggtgaagggcctgaggaacaagttccagctgaactgctacgtgaagattaacaagaacaagcctattatttacattgattctatgtcttacctgattttctacaacctgattaagccttacctgattcctcagatgatgtacaagctgcctaacaccatctcttctgagaccttcctgaagtga

SEQ ID NO: 23 - DNA, 2028 bp, Artificial Sequence
  Insert of SAH54-4
atgggtaagaacattaagaagaaccaggtgatgaacctgggccctaactctaagctgcttaaggaatacaagtctcagctgattgagctgaacattgagcagttcgaggctggcataggcctgattctgggcgatgcttacattaggtctagggatgagggcaagacctactgcatgcagttcgagtggaagaacaaggcttacatggatcacgtgtgcctgctgtacgatcagtgggtgctgtctcctcctcacaagaaggagagggtgaaccacttgggaaacctggtgattacctggggcgctcaaaccttcaagcaccaggctttcaacaagctggctaacctgttcattgtgaacaacaagaagaccattcctaacaacctggtggagaactacctgacccctatgtctctggcttactggttcatggatgatggcggcaagtgggattacaacaagaactctaccaacaagtctattgtgctgaacacccagtctttcaccttcgaggaggtggaatacctggtgaagggcctgaggaacaagttccagctgaactgctacgtgaagattaacaagaacaagcctattatttacattgattctatgtcttacctgattttctacaacctgattaagccttacctgattcctcagatgatgtacaagctgcctaacaccatctcttctgagaccttcctgaagcttagtagactcgataagtctaaggtgatcaactctgctcttgagcttcttaacgaagtgggaatcgagggactcactactagaaagctcgctcagaagttgggagttgagcagcctactctttactggcacgtgaagaacaagagagctttgcttgacgctctcgctatcgagatgctcgatagacatcacactcatttctgccctcttgagggagaatcttggcaggatttcctcagaaacaacgctaagtctttcagatgcgctctcctctctcatagagatggtgctaaggttcaccttggaactagacctactgagaagcagtacgagactcttgaaaaccagcttgctttcctttgtcagcagggattctctcttgagaacgctctttacgctttgtctgctgtgggacatttcactcttggatgcgtgcttgaggatcaagagcatcaggtggcaaaagaagagagagagactcctactactgattctatgcctcctcttctcagacaagctatcgagcttttcgatcatcaaggtgctgaacctgctttccttttcggactcgaacttatcatctgcggacttgagaagcaacttaagtgcgagtctggatcttctggtggtggaggatctggtggaggtggaagtggaggtggtggatcaggtggtggaggtagcggaggtggaggaatgtcaagattggacaagagcaaggtcatcaacagcgctcttgagttgctcaatgaagttggtattgagggacttacaaccaggaagttggctcaaaagctcggtgttgagcagccaacactctattggcatgtcaagaacaagcgtgctctccttgatgctttggccatcgagatgcttgatcgtcaccatacccacttttgtcctcttgagggggagagctggcaagatttccttcgtaacaacgccaagtcattccgttgtgctttgctctcacatcgtgatggtgctaaagtgcatctcggtacaagaccaaccgagaagcaatacgaaacattggagaaccagttggctttcttgtgtcaacaggggttctcactcgaaaatgccctttacgctctctcagctgttggtcacttcactctcggttgcgttttggaggatcaggaacaccaagtcgctaaagaggaaagagagacacctacaaccgatagcatgcctcctttgttgagacaggccatcgagttgtttgaccatcaaggtgccgagccagcttttttgttcgggcttgagcttatcatttgcgggctcgaaaagcagttgaagtgcgagtcaggttcttga

SEQ ID NO: 24 - DNA, 2049 bp, Artificial Sequence
  Insert of VC-SAH53-10
atgggtcctaagaagaagagaaaggttaagaacattaagaagaaccaggtgatgaacctgggccctaactctaagctgcttaaggaatacaagtctcagctgattgagctgaacattgagcagttcgaggctggcataggcctgattctgggcgatgcttacattaggtctagggatgagggcaagacctactgcatgcagttcgagtggaagaacaaggcttacatggatcacgtgtgcctgctgtacgatcagtgggtgctgtctcctcctcacaagaaggagagggtgaaccacttgggaaacctggtgattacctggggcgctcaaaccttcaagcaccaggctttcaacaagctggctaacctgttcattgtgaacaacaagaagaccattcctaacaacctggtggagaactacctgacccctatgtctctggcttactggttcatggatgatggcggcaagtgggattacaacaagaactctaccaacaagtctattgtgctgaacacccagtctttcaccttcgaggaggtggaatacctggtgaagggcctgaggaacaagttccagctgaactgctacgtgaagattaacaagaacaagcctattatttacattgattctatgtcttacctgattttctacaacctgattaagccttacctgattcctcagatgatgtacaagctgcctaacaccatctcttctgagaccttcctgaagcttagtagactcgataagtctaaggtgatcaactctgctcttgagcttcttaacgaagtgggaatcgagggactcactactagaaagctcgctcagaagttgggagttgagcagcctactctttactggcacgtgaagaacaagagagctttgcttgacgctctcgctatcgagatgctcgatagacatcacactcatttctgccctcttgagggagaatcttggcaggatttcctcagaaacaacgctaagtctttcagatgcgctctcctctctcatagagatggtgctaaggttcaccttggaactagacctactgagaagcagtacgagactcttgaaaaccagcttgctttcctttgtcagcagggattctctcttgagaacgctctttacgctttgtctgctgtgggacatttcactcttggatgcgtgcttgaggatcaagagcatcaggtggcaaaagaagagagagagactcctactactgattctatgcctcctcttctcagacaagctatcgagcttttcgatcatcaaggtgctgaacctgctttccttttcggactcgaacttatcatctgcggacttgagaagcaacttaagtgcgagtctggatcttctggtggtggaggatctggtggaggtggaagtggaggtggtggatcaggtggtggaggtagcggaggtggaggaatgtcaagattggacaagagcaaggtcatcaacagcgctcttgagttgctcaatgaagttggtattgagggacttacaaccaggaagttggctcaaaagctcggtgttgagcagccaacactctattggcatgtcaagaacaagcgtgctctccttgatgctttggccatcgagatgcttgatcgtcaccatacccacttttgtcctcttgagggggagagctggcaagatttccttcgtaacaacgccaagtcattccgttgtgctttgctctcacatcgtgatggtgctaaagtgcatctcggtacaagaccaaccgagaagcaatacgaaacattggagaaccagttggctttcttgtgtcaacaggggttctcactcgaaaatgccctttacgctctctcagctgttggtcacttcactctcggttgcgttttggaggatcaggaacaccaagtcgctaaagaggaaagagagacacctacaaccgatagcatgcctcctttgttgagacaggccatcgagttgtttgaccatcaaggtgccgagccagcttttttgttcgggcttgagcttatcatttgcgggctcgaaaagcagttgaagtgcgagtcaggttcttga

SEQ ID NO: 25 - DNA, 1152 bp, Artificial Sequence
  Insert of VC-SAH28-5
atgggaggaatgtctaagatgcctcagttcaaccttagatggcctagagaggttctcgatcttgttagaaaggtggcagaagagaacggaagatcagtgaactctgaaatctaccagagggtgatggaatctttcaagaaagagggaaggattggagcaggtggaggatctggtggaggaacaggtggtggttctggtggtggaatgaagggaatgagtaaaatgccacaattcaacctcaggtggccaagggaagttttggatcttgtgaggaaagtggctgaggaaaacggaagaagtgtgaactcagagatataccaaagagttatggaaagttttaagaaagagggtagaatcggagctagatctggtggtggtagtggaggtggaactggtggaggttcaggtggaggcgcccctaagaagaagagaaaggttctcgagatgaagaacattaagaagaaccaggtgatgaacctgggccctaactctaagctgcttaaggaatacaagtctcagctgattgagctgaacattgagcagttcgaggctggcataggcctgattctgggcgatgcttacattaggtctagggatgagggcaagacctactgcatgcagttcgagtggaagaacaaggcttacatggatcacgtgtgcctgctgtacgatcagtgggtgctgtctcctcctcacaagaaggagagggtgaaccacttgggaaacctggtgattacctggggcgctcaaaccttcaagcaccaggctttcaacaagctggctaacctgttcattgtgaacaacaagaagaccattcctaacaacctggtggagaactacctgacccctatgtctctggcttactggttcatggatgatggcggcaagtgggattacaacaagaactctaccaacaagtctattgtgctgaacacccagtctttcaccttcgaggaggtggaatacctggtgaagggcctgaggaacaagttccagctgaactgctacgtgaagattaacaagaacaagcctattatttacattgattctatgtcttacctgattttctacaacctgattaagccttacctgattcctcagatgatgtacaagctgcctaacaccatctcttctgagaccttcctgaagtga

SEQ ID NO: 26 - DNA, 1107 bp, Artificial Sequence
  Insert of VC-SAH46-4
atgggaggaatgtctaagatgcctcagttcaaccttagatggcctagagaggttctcgatcttgttagaaaggtggcagaagagaacggaagatcagtgaactctgaaatctaccagagggtgatggaatctttcaagaaagagggaaggattggagcaggtggaggatctggtggaggaacaggtggtggttctggtggtggaatgaagggaatgagtaaaatgccacaattcaacctcaggtggccaagggaagttttggatcttgtgaggaaagtggctgaggaaaacggaagaagtgtgaactcagagatataccaaagagttatggaaagttttaagaaagagggtagaatcggagctagatccgcccctaagaagaagagaaaggttctcgagatgaagaacattaagaagaaccaggtgatgaacctgggccctaactctaagctgcttaaggaatacaagtctcagctgattgagctgaacattgagcagttcgaggctggcataggcctgattctgggcgatgcttacattaggtctagggatgagggcaagacctactgcatgcagttcgagtggaagaacaaggcttacatggatcacgtgtgcctgctgtacgatcagtgggtgctgtctcctcctcacaagaaggagagggtgaaccacttgggaaacctggtgattacctggggcgctcaaaccttcaagcaccaggctttcaacaagctggctaacctgttcattgtgaacaacaagaagaccattcctaacaacctggtggagaactacctgacccctatgtctctggcttactggttcatggatgatggcggcaagtgggattacaacaagaactctaccaacaagtctattgtgctgaacacccagtctttcaccttcgaggaggtggaatacctggtgaagggcctgaggaacaagttccagctgaactgctacgtgaagattaacaagaacaagcctattatttacattgattctatgtcttacctgattttctacaacctgattaagccttacctgattcctcagatgatgtacaagctgcctaacaccatctcttctgagaccttcctgaagtga

SEQ ID NO: 27 - DNA, 4905 bp, Artificial Sequence
  Construct II
agcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagnnnnnnnnnnaagcttgactctcttaagggagcgtcgagtacgcgcccggggagcccaagggcacgccctggcacccgaagctctagtatcaaatttggcacaaaaagcaaaattaaaatactgataattgccaacacaattaacatctcaatcaaggtaaatgctttttgctttttttgccaaagctatcttccgtgatcagagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactctagtgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggc

SEQ ID NO: 28 - DNA, 260 bp, Artificial Sequence
  Insert of VC-SAH6-1
ttgccatgttttacggcagtgagagcagagatagcgctgatgtccggcggtgcttttgccgttacgcaccaccccgtcagtagctgaacaggagggacagctggcgaaagggggatgtgctgcaaggcgattaagttgggtaacgccagggttttcccagtcacgacgttgtaaaacgacggccagtgagcgcgcgtaatacgactcactatagggcgaattgggtactcgagtacgctagggataacagggtaatatag

SEQ ID NO: 29 - DNA, 4580 bp, Artificial Sequence
  VC-SAH7-1
ctagtgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttact

SEQ ID NO: 30 - DNA, 38 bp, Artificial Sequence
  Insert of VX-SAH60-5
ctatcaatgatagcgctagggataacagggtaatatag

SEQ ID NO: 31 - DNA, 39 bp, Artificial Sequence
  Insert of VX-SAH61-1
ctatcaatgatagacgctagggataacagggtaatatag

SEQ ID NO: 32 - DNA, 40 bp, Artificial Sequence
  Insert of VX-SAH62-1
ctatcaatgatagtacgctagggataacagggtaatatag

SEQ ID NO: 33 - DNA, 5221 bp, Artificial Sequence
  Construct III
agcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtggttacagtcttgcgcgacatgcgtcaccacggtgatatcgtccacccaggtgttcggcgtggtgtagagcattacgctgcgatggattccggcatagttaaagaaatcatggaagtaagactgctttttcttgccgttttcgtcggtaatcaccattcccggcgggatagtctgccagttcagttcgttgttcacacaaacggtgatacgtacacttttcccggcaataacatacggcgtgacatcggcttcaaatggcgtatagccgccctgatgctccatcacttcctgattattgacccacactttgccgtaatgagtgaccgcatcgaaacgcagcacgatacgctggcctgcccaacctttcggtataaagacttcgcgctgataccagacgttgcccgcataattacgaatatctgcatcggcgaactgatcgttaaaactgcctggcacagcaattgcccggctttcttgtaacgcgctttcccaccaacgctgatcaattccacagttttcgcggtccagactgaatgcccacaggccgtcgagttttttgatttcacgggttggggtttctacaggactctagannnnnnnnnngcggccgctggcaccacctgccagtcaacagacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactctagtgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggc

SEQ ID NO: 34 - DNA, 35 bp, Artificial Sequence
  Insert of VX-SAH132
tagggataacagggtaatactagtagagtgcctag

SEQ ID NO: 35 - DNA, 36 bp, Artificial Sequence
  Insert of VX-SAH1133
tagggataacagggtaatacttagtagagtgcctag

SEQ ID NO: 36 - DNA, 37 bp, Artificial Sequence
  Insert of VX-SAH134
tagggataacagggtaatactatagtagagtgcctag

SEQ ID NO: 37 - DNA, 38 bp, Artificial Sequence
  Insert of VX-SAH135
tagggataacagggtaatactagtagtagagtgcctag

SEQ ID NO: 38 - DNA, 8885 bp, Artificial Sequence
  Construct IV
ccnnnnnnnnnnttaattaacgaagagcaagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgcctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaagcttggacaatcagtaaattgaacggagaatattattcataaaaatacgatagtaacgggtgatatattcattagaatgaaccgaaaccggcggtaaggatctgagctacacatgctcaggttttttacaacgtgcacaacagaattgaaagcaaatatcatgcgatcataggcgtctcgcatatctcattaaagcagggcatgccggtcgagtcaaatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatggtagactcgacggatccacgtgtggaagatatgaatttttttgagaaactagataagattaatgaatatcggtgttttggttttttcttgtggccgtctttgtttatattgagatttttcaaatcagtgcgcaagacgtgacgtaagtatccgagtcagtttttatttttctactaatttggtcgaagctttgggcggatcctctagaattcgaatccaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagcccgggttgctctt

SEQ ID NO: 39 - DNA, 10934 bp, Artificial Sequence
  Construct V
gtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtgccnnnnnnnnnnctagagtcctgtagaaaccccaacccgtgaaatcaaaaaactcgacggcctgtgggcattcagtctggaccgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaagcggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaatcaacaactctcctggcgcaccatcgtcggctacagcctcgggaattgctaccgagctcgaatttccccgatcgttcaaacatttggcaataaagtttcttaagattgaatcctgttgccggacttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggaataagcttggcgtaatcatggtcatagctgtttcctactagatctgattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtccatggaacgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctagctagaaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtaacgcgtggtgattttgtgccgagctgccggtcggggagctgttggctggctggtggcaggatatattgtggtgtaaacaaattgacgcttagacaacttaataacacattgcggacgtctttaatgtactgaattaacatccgtttgatacttgtctaaaattggctgatttcgagtgcatctatgcataaaaacaatctaatgacaattattaccaagcaggatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaccggatctggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggacccaagctctagatcttgctgcgttcggatattttcgtggagttcccgccacagacccggatgatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggcctcctgtcaagctctgagtcgttgtaaaacgacggccagtgaattgagctcggtaccgagtcaaagattcaaatagaggacctaacagaactcgccgtaaagactggcgaacagttcatacagagtctcttacgactcaatgacaagaagaaaatcttcgtcaacatggtggagcacgacacgcttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacagggtacgtacctagaatacaaagaagaggaagaagaaacctctacagaagaaagtgatggatccccgggatcatctacttctgaagactcagactcagactaagcaggtgacgaacgtcaccaatcccaattcgatctacatccgtcct

SEQ ID NO: 40 - DNA, 202 bp, Artificial Sequence
  Insert of VX-SAH13
agcggccgcctagggatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgcattaccctgttatccctagcgctatcattgatagttatccctat

SEQ ID NO: 41 - DNA, 203 bp, Artificial Sequence
  Insert of VX-SAH114
agcggccgcctagggatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgcattaccctgttatccctagcgtctatcattgatagttatccctat

SEQ ID NO: 42 - DNA, 204 bp, Artificial Sequence
  Insert of VX-SAH115
agcggccgcctagggatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgcattaccctgttatccctagcgtactatcattgatagttatccctat

SEQ ID NO: 43 - DNA, 44 bp, Artificial Sequence
  Insert of VX-SAH16
acggccgcctaggcactctactagtattaccctgttatccctat

SEQ ID NO: 44 - DNA, 45 bp, Artificial Sequence
  Insert of VX-SAH17
acggccgcctaggcactctactaagtattaccctgttatccctat

SEQ ID NO: 45 - DNA, 46 bp, Artificial Sequence
  Insert of VX-SAH18
acggccgcctaggcactctactatagtattaccctgttatccctat

SEQ ID NO: 46 - DNA, 47 bp, Artificial Sequence
  Insert of VC-SAH19
acggccgcctaggcactctactactagtattaccctgttatccctat

SEQ ID NO: 47 - DNA, 693 bp, Artificial Sequence
  Insert of VC-SAH44-32
atgggacaggtgatgaacctgggccctaactctaagctgcttaaggaatacaagtctcagctgattgagctgaacattgagcagttcgaggctggcataggcctgattctgggcgatgcttacattaggtctagggatgagggcaagacctactgcatgcagttcgagtggaagaacaaggcttacatggatcacgtgtgcctgctgtacgatcagtgggtgctgtctcctcctcacaagaaggagagggtgaaccacttgggaaacctggtgattacctggggcgctcaaaccttcaagcaccaggctttcaacaagctggctaacctgttcattgtgaacaacaagaagaccattcctaacaacctggtggagaactacctgacccctatgtctctggcttactggttcatggatgatggcggcaagtgggattacaacaagaactctaccaacaagtctattgtgctgaacacccagtctttcaccttcgaggaggtggaatacctggtgaagggcctgaggaacaagttccagctgaactgctacgtgaagattaacaagaacaagcctattatttacattgattctatgtcttacctgattttctacaacctgattaagccttacctgattcctcagatgatgtacaagctgcctaacaccatctcttctgagaccttcctgaagtga

SEQ ID NO: 48 - DNA, 684 bp, Artificial Sequence
  Insert of VX-SAH43-8
atgggtaagaacattaagaagaaccaggtgatgaacctgggccctaactctaagctgcttaaggaatacaagtctcagctgattgagctgaacattgagcagttcgaggctggcataggcctgattctgggcgatgcttacattaggtctagggatgagggcaagacctactgcatgcagttcgagtggaagaacaaggcttacatggatcacgtgtgcctgctgtacgatcagtgggtgctgtctcctcctcacaagaaggagagggtgaaccacttgggaaacctggtgattacctggggcgctcaaaccttcaagcaccaggctttcaacaagctggctaacctgttcattgtgaacaacaagaagaccattcctaacaacctggtggagaactacctgacccctatgtctctggcttactggttcatggatgatggcggcaagtgggattacaacaagaactctaccaacaagtctattgtgctgaacacccagtctttcaccttcgaggaggtggaatacctggtgaagggcctgaggaacaagttccagctgaactgctacgtgaagattaacaagaacaagcctattatttacattgattctatgtcttacctgattttctacaacctgattaagccttacctgattcctcagatgatgtacaagctgcctaactga

SEQ ID NO: 49 - DNA, 705 bp, Artificial Sequence
  Inset of VC-SAH42-13
atgggtcctaagaagaagagaaaggttaagaacattaagaagaaccaggtgatgaacctgggccctaactctaagctgcttaaggaatacaagtctcagctgattgagctgaacattgagcagttcgaggctggcataggcctgattctgggcgatgcttacattaggtctagggatgagggcaagacctactgcatgcagttcgagtggaagaacaaggcttacatggatcacgtgtgcctgctgtacgatcagtgggtgctgtctcctcctcacaagaaggagagggtgaaccacttgggaaacctggtgattacctggggcgctcaaaccttcaagcaccaggctttcaacaagctggctaacctgttcattgtgaacaacaagaagaccattcctaacaacctggtggagaactacctgacccctatgtctctggcttactggttcatggatgatggcggcaagtgggattacaacaagaactctaccaacaagtctattgtgctgaacacccagtctttcaccttcgaggaggtggaatacctggtgaagggcctgaggaacaagttccagctgaactgctacgtgaagattaacaagaacaagcctattatttacattgattctatgtcttacctgattttctacaacctgattaagccttacctgattcctcagatgatgtacaagctgcctaactga

SEQ ID NO: 50 - DNA, 666 bp, Artificial Sequence
  Insert of VC-SAH45-3
atgggacaggtgatgaacctgggccctaactctaagctgcttaaggaatacaagtctcagctgattgagctgaacattgagcagttcgaggctggcataggcctgattctgggcgatgcttacattaggtctagggatgagggcaagacctactgcatgcagttcgagtggaagaacaaggcttacatggatcacgtgtgcctgctgtacgatcagtgggtgctgtctcctcctcacaagaaggagagggtgaaccacttgggaaacctggtgattacctggggcgctcaaaccttcaagcaccaggctttcaacaagctggctaacctgttcattgtgaacaacaagaagaccattcctaacaacctggtggagaactacctgacccctatgtctctggcttactggttcatggatgatggcggcaagtgggattacaacaagaactctaccaacaagtctattgtgctgaacacccagtctttcaccttcgaggaggtggaatacctggtgaagggcctgaggaacaagttccagctgaactgctacgtgaagattaacaagaacaagcctattatttacattgattctatgtcttacctgattttctacaacctgattaagccttacctgattcctcagatgatgtacaagctgcctaactga

SEQ ID NO: 51 - DNA, 708 bp, Artificial Sequence
  Insert of VC-SAH105
atgaagaacattaagaagaaccaggtgatgaacctgggccctaactctaagctgcttaaggaatacaagtctcagctgattgagctgaacattgagcagttcgaggctggcataggcctgattctgggcgatgcttacattaggtctagggatgagggcaagacctactgcatgcagttcgagtggaagaacaaggcttacatggatcacgtgtgcctgctgtacgatcagtgggtgctgtctcctcctcacaagaaggagagggtgaaccacttgggaaacctggtgattacctggggcgctcaaaccttcaagcaccaggctttcaacaagctggctaacctgttcattgtgaacaacaagaagaccattcctaacaacctggtggagaactacctgacccctatgtctctggcttactggttcatggatgatggcggcaagtgggattacaacaagaactctaccaacaagtctattgtgctgaacacccagtctttcaccttcgaggaggtggaatacctggtgaagggcctgaggaacaagttccagctgaactgctacgtgaagattaacaagaacaagcctattatttacattgattctatgtcttacctgattttctacaacctgattaagccttacctgattcctcagatgatgtacaagctgcctaacaccatctcttctgagaccttcctgaagtga

SEQ ID NO: 52 - DNA, 679 bp, Artificial Sequence
  Insert of VC-SAH106
atgaagaacattaagaagaaccaggtgatgaacctgggccctaactctaagctgcttaaggaatacaagtctcagctgattgagctgaacattgagcagttcgaggctggcataggcctgattctgggcgatgcttacattaggtctagggatgagggcaagacctactgcatgcagttcgagtggaagaacaaggcttacatggatcacgtgtgcctgctgtacgatcagtgggtgctgtctcctcctcacaagaaggagagggtgaaccacttgggaaacctggtgattacctggggcgctcaaaccttcaagcaccaggctttcaacaagctggctaacctgttcattgtgaacaacaagaagaccattcctaacaacctggtggagaactacctgacccctatgtctctggcttactggttcatggatgatggcggcaagtgggattacaacaagaactctaccaacaagtctattgtgctgaacacccagtctttcaccttcgaggaggtggaatacctggtgaagggcctgaggaacaagttccagctgaactgctacgtgaagattaacaagaacaagcctattatttacattgattctatgtcttacctgattttctacaacctgattaagccttacctgattcctcagatgatgtacaagctgcctaact

SEQ ID NO: 53 - DNA, 6527 bp, Artificial Sequence
  pGBT9
gcttgcatgcaacttcttttctttttttttcttttctctctcccccgttgttgtctcaccatatccgcaatgacaaaaaaatgatggaagacactaaaggaaaaaattaacgacaaagacagcaccaacagatgtcgttgttccagagctgatgaggggtatctcgaagcacacgaaactttttccttccttcattcacgcacactactctctaatgagcaacggtatacggccttccttccagttacttgaatttgaaataaaaaaaagtttgctgtcttgctatcaagtataaatagacctgcaattattaatcttttgtttcctcgtcattgttctcgttccctttcttccttgtttctttttctgcacaatatttcaagctataccaagcatacaatcaactccaagcttgaagcaagcctcctgaaagatgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcgccggaattcccggggatccgtcgacctgcagccaagctaattccgggcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccggcaagtgcacaaacaatacttaaataaatactactcagtaataacctatttcttagcatttttgacgaaatttgctattttgttagagtcttttacaccatttgtctccacacctccgcttacatcaacaccaataacgccatttaatctaagcgcatcaccaacattttctggcgtcagtccaccagctaacataaaatgtaagctttcggggctctcttgccttccaacccagtcagaaatcgagttccaatccaaaagttcacctgtcccacctgcttctgaatcaaacaagggaataaacgaatgaggtttctgtgaagctgcactgagtagtatgttgcagtcttttggaaatacgagtcttttaataactggcaaaccgaggaactcttggtattcttgccacgactcatctccatgcagttggacgatatcaatgccgtaatcattgaccagagccaaaacatcctccttaggttgattacgaaacacgccaaccaagtatttcggagtgcctgaactatttttatatgcttttacaagacttgaaattttccttgcaataaccgggtcaattgttctctttctattgggcacacatataatacccagcaagtcagcatcggaatctagagcacattctgcggcctctgtgctctgcaagccgcaaactttcaccaatggaccagaactacctgtgaaattaataacagacatactccaagctgcctttgtgtgcttaatcacgtatactcacgtgctcaatagtcaccaatgccctccctcttggccctctccttttcttttttcgaccgaattaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccaggaagatccgaggcctagcttctaattcttccaacatacaatgggagtttggccgagtggtttaaggcgtcagatttaggtggatttaacctctaaaatctctgatatcttcggatgcaagggttcgaatcccttagctctcattattttttgctttttctcttgaggtcacatgatcgcaaaatggcaaatggcacgtgaagctgtcgatattggggaactgtggtggttggcaaatgactaattaagttagtcaaggcgccatcctcatgaaaactgtgtaacataataaccgaagtgtcgaaaaggtggcaccttgtccaattgaacacgctcgatgaaaaaaataagatatatataaggttaagtaaagcgtctgttagaaaggaagtttttcctttttcttgctctcttgtcttttcatctactatttccttcgtgtaatacagggtcgtcagatacatagatacaattctattacccccatccatacaatgggccatatggcttctagctatccttatgacgtgcctgactatgccagcctgggaggaccttctagtcctaagaagaagagaaaggtggcggccgcattagcccgaagatcttcgggctgatctcccatgtctctactggtggtggtgcttctttggaattattggaaggtaaggaattgccaggtgttgctttcttatccgaaaagaaataaattgaattgaattgaaatcgatagatcaatttttttcttttctctttccccatcctttacgctaaaataatagtttattttattttttgaatattttttatttatatacgtatatatagactattatttatcttttaatgattattaagatttttattaaaaaaaaattcgctcctcttttaatgcctttatgcagtttttttttcccattcgatatttctatgttcgggttcagcgtattttaagtttaataactcgaaaattctgcgttcgttaaagctaggcctcggatcttcctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgtt

SEQ ID NO: 54 - DNA, 11312 bp, Artificial Sequence
  Construct VI
aaagttgccatgattacgccaagcttgactagagaattcgaatccaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagcccggatcccgccaccnnnnnnnnnnctcgagcatgcaggcatgccctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtccaggtaccccactttgtacaagaaagctgggtccatgattagccaagcttgcatgccgtcgaccagatctgatatctgcggccgcctcgagcatatgctagaggatccccgggtaccagcctgcttttttgtacaaacttgccatgattacgccaagcttgcatgccgatcccccccactccgccctacactcgtatatatatgcctaaacctgccccgttcctcatatgtgatattattatttcattattaggtataagatagtaaacgataaggaaagacaatttattgagaaagccatgctaaaatatagatagatataccttagcaggtgtttattttacaacataacataacatagtagctagccagcaggcaggctaaaacatagtatagtctatctgcagggggtacggtcgactctagactagagtcgcggccgctacaggaacaggtggtggcggccctcggtgcgctcgtactgctccacgatggtgtagtcctcgttgtgggaggtgatgtccagcttggcgtccacgtagtagtagccgggcagctgcacgggcttcttggccatgtagatggacttgaactccaccaggtagtggccgccgtccttcagcttcagggccttgtgggtctcgcccttcagcacgccgtcgcgggggtacaggcgctcggtggaggcctcccagcccatggtcttcttctgcatcacggggccgtcggaggggaagttcacgccgatgaacttcaccttgtagatgaagcagccgtcctgcagggaggagtcctgggtcacggtcgccacgccgccgtcctcgaagttcatcacgcgctcccacttgaagccctcggggaaggacagcttcttgtagtcggggatgtcggcggggtgcttcacgtacaccttggagccgtactggaactggggggacaggatgtcccaggcgaagggcagggggccgcccttggtcaccttcagcttcacggtgttgtggccctcgtaggggcggccctcgccctcgccctcgatctcgaactcgtggccgttcacggtgccctccatgcgcaccttgaagcgcatgaactcggtgatgacgttctcggaggaggccatggtggcgaccgggggctcgactctagatgaaatcgaaattcagagttttgatagtgagagcaaagagggacggacttatgaggatttcgagtatttcaagagatggtacttgttgatcggacggctacgatgatctcgatttggttaatccagtatctcgcggtgtatggagttatggtagggttaatggtcaatttcatctaacggtagagaatgatgtaattagataagaatcttgagatactggtttagattggatgagtgtagggtccatcttatcttgataagtggatggtttttagagacacagtgaatattagccaatcgaagttccatatcaccatcatcatctgtataattttgtttttttggaagataataatgattgaaattttggtagattttatttttcattatttaccttgtatgttgagtggtcttcaaattattgaacgtgacagattcacaagaaagtagattttttataaatgaaattttacttattttaaaggtatctctatttaatttcttttgtttatggttgtctgtcagcatttgacttgcagtttcatgctcatagtcatatacgttattctaggcttttttgaatatcttattacttttttcgtaatacaattttataattttatcaaagttatacaactataactaaaattagggttttctacaaaacaaaaaaatcttctaattttttttgttgtagccagtttactcgtaagttacaaaaaaatacaaatgaacccacatgtattatgcgtttaactaggattaccatgtactttcatgtactcaattcaccctatactctttttttttttttttctagttccacccaatctataaaattctgtccatttgaccaaattcaattaatttctgtaattgcgatttaaaattaatattacatgttcactatttctcgatttgagggaacccgagtttaaatatgataaaaatgttgacccatcactacaaatatgttatagtttatacttaatagtggtgtttttggggataattgatgaattaagtaaacatgattcttcttatgaagttgattgagtgattattgtatgtaaacctatgtgattgatgttattggttgattgagtgattattgtattagtatgtaagcaaagatgattgttcttatgaggtaatttgttactcattcatccttttgcatatgagaaattgtgttagcgtacgcaaaacaatagagaacataaaagatatgtgtatttatttaaggtgacttttgttaatgatattgtagtatctatacatttatatataacttgttgaatttgagtataagctatcaggatccgggggatcctctagagtcgaggtacccaacttttctatacaaagttgatagcttggcgtaatcgatagcttggcgtaatcatggtcatagctgtttcctactagatctgattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtccatggaacgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgggggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctagctagaaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtaacgcgtggtgattttgtgccgagctgccggtcggggagctgttggctggctggtggcaggatatattgtggtgtaaacaaattgacgcttagacaacttaataacacattgcggacgtctttaatgtactgaattaacatccgtttgatacttgtctaaaattggctgatttcgagtgcatctatgcataaaaacaatctaatgacaattattaccaagcagagcttgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatctagagcttgggtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatcctgagtcgttgtaaaacgacggccagtgaattatccggccagtgaattatcaactatgtataat

SEQ ID NO: 55 - DNA, 10765 bp, Artificial Sequence
  VC-SCB583-40
ttccatggacatacaaatggacgaacggataaaccttttcacgcccttttaaatatccgattattctaataaacgctcttttctcttaggtttacccgccaatatatcctgtcaaacactgatagtttaaactgaaggcgggaaacgacaatcagatctagtaggaaacagctatgaccatgattacgccaagcttattccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagtccggcaacaggattcaatcttaagaaactttattgccaaatgtttgaacgatcggggaaattcgagctcggtagcaattcccgaggctgtagccgacgatggtgcgccaggagagttgttgattcattgtttgcctccctgctgcggtttttcaccgaagttcatgccagtccagcgtttttgcagcagaaaagccgccgacttcggtttgcggtcgcgagtgaagatccctttcttgttaccgccaacgcgcaatatgccttgcgaggtcgcaaaatcggcgaaattccatacctgttcaccgacgacggcgctgacgcgatcaaagacgcggtgatacatatccagccatgcacactgatactcttcactccacatgtcggtgtacattgagtgcagcccggctaacgtatccacgccgtattcggtgatgataatcggctgatgcagtttctcctgccaggccagaagttctttttccagtaccttctctgccgcttccaaatcgccgctttggacataccatccgtaataacggttcaggcacagcacatcaaagagatcgctgatggtatcggtgtgagcgtcgcagaacattacattgacgcaggtgatcggacgcgtcgggtcgagtttacgcgttgcttccgccagtggcgcgaaatattcccgtgcaccttgcggacgggtatccggttcgttggcaatactccacatcaccacgcttgggtggtttttgtcacgcgctatcagctctttaatcgcctgtaagtgcgcttgctgagtttccccgttgactgcctcttcgctgtacagttctttcggcttgttgcccgcttcgaaaccaatgcctaaagagaggttaaagccgacagcagcagtttcatcaatcaccacgatgccatgttcatctgcccagtcgagcatctcttcagcgtaagggtaatgcgaggtacggtaggagttggccccaatccagtccattaatgcgtggtcgtgcaccatcagcacgttatcgaatcctttgccacgcaagtccgcatcttcatgacgaccaaagccagtaaagtagaacggtttgtggttaatcaggaactgttcgcccttcactgccactgaccggatgccgacgcgaagcgggtagatatcacactctgtctggcttttggctgtgacgcacagttcatagagataaccttcacccggttgccagaggtgcggattcaccacttgcaaagtcccgctagtgccttgtccagttgcaaccacctgttgatccgcatcacgcagttcaacgctgacatcaccattggccaccacctgccagtcaacagacgcgtggttacagtcttgcgcgacatgcgtcaccacggtgatatcgtccacccaggtgttcggcgtggtgtagagcattacgctgcgatggattccggcatagttaaagaaatcatggaagtaagactgctttttcttgccgttttcgtcggtaatcaccattcccggcgggatagtctgccagttcagttcgttgttcacacaaacggtgatacgtacacttttcccggcaataacatacggcgtgacatcggcttcaaatggcgtatagccgccctgatgctccatcacttcctgattattgacccacactttgccgtaatgagtgaccgcatcgaaacgcagcacgatacgctggcctgcccaacctttcggtataaagacttcgcgctgataccagacgttgcccgcataattacgaatatctgcatcggcgaactgatcgttaaaactgcctggcacagcaattgcccggctttcttgtaacgcgctttcccaccaacgctgatcaattccacagttttcgcggtccagactgaatgcccacaggccgtcgagttttttgatttcacgggttggggtttctacaggactctagctggcacagagttaccgggtgaatttcgctaccttaggatcgtaaccaatatgtctcacggcgttttcggactagactattaccctgttatccctaggcggccgctggcaccacctgccagtcaacagacgcgtggttacagtcttgcgcgacatgcgtcaccacggtgatatcgtccacccaggtgttcggcgtggtgtagagcattacgctgcgatggattccggcatagttaaagaaatcatggaagtaagactgctttttcttgccgttttcgtcggtaatcaccattcccggcgggatagtctgccagttcagttcgttgttcacacaaacggtgatacgtacacttttcccggcaataacatacggcgtgacatcggcttcaaatggcgtatagccgccctgatgctccatcacttcctgattattgacccacactttgccgtaatgagtgaccgcatcgaaacgcagcacgatacgctggcctgcccaacctttcggtataaagacttcgcgctgataccagacgttgcccgcataattacgaatatctgcatcggcgaactgatcgttaaaactgcctggcacagcaattgcccggctttcttgtaacgcgctttcccaccaacgctgatcaattccacagttttcgcgatccagactgaatgcccacaggccgtcgagttttttgatttcacgggttggggtttctacaggacggatgtagatcgaattgggattggtgacgttcgtcacctgcttagtctgagtctgagtcttcagaagtagatgatcccggggatccatcactttcttctgtagaggtttcttcttcctcttctttgtattctaggtacgtaccctgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagcgtgtcgtgctccaccatgttgacgaagattttcttcttgtcattgagtcgtaagagactctgtatgaactgttcgccagtctttacggcgagttctgttaggtcctctatttgaatctttgactcggtaccgagctcgaattcactggccgtcgttttacaacgactcagccagcttgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatcggtcgatcgactcagatctgggtaactggcctaactggccttggaggagctggcaactcaaaatccctttgccaaaaaccaacatcatgccatccaccatgcttgtatccagccgcgcgcaatgtaccccgcgctgtgtatcccaaagcctcatgcaacctaacagatggatcgtttggaaggcctataacagcaaccacagacttaaaaccttgcgcctccatagacttaagcaaatgtgtgtacaatgtagatcctaggcccaacctttgatgcctatgtgacacgtaaacagtactctcaactgtccaatcgtaagcgttcctagccttccagggcccagcgtaagcaataccagccacaacaccctcaacctcagcaaccaaccaagggtatctatcttgcaacctctctaggtcatcaatccactcttgtggtgtttgtggctctgtcctaaagttcactgtagacgtctcaatgtaatggttaacgatgtcacaaaccgcggccatatcggctgctgtagctggcctaatctcaactggtctcctctccggagacatgtcgagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatcctgcttggtaataattgtcattagattgtttttatgcatagatgcactcgaaatcagccaattttagacaagtatcaaacggatgttaattcagtacattaaagacgtccgcaatgtgttattaagttgtctaagcgtcaatttgtttacaccacaatatatcctgccaccagccagccaacagctccccgaccggcagctcggcacaaaatcaccacgcgttaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaatttctagctagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttcccccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcg

SEQ ID NO: 56 - protein, 235 aa, Saccharomyces cerevisiae
MKNIKKNQVMNTGPNSKLLKEYKSQLIELNIEQFEAGIGLILGDAYIRSRDEGKTYCMQFEWKNKAYMDHVCLLYDQWVLSPPHKKERVNHLGNLVITWGAQTFKHQAFNKLANLFIVNNKKTIPNNLVENYLTPMSTAYWFMDDGGKWDYNKNSTNKSIVLNTQSFTFEEVEYLVKGLRNKFQLNCYVKINKNKPIIYIDSMSYTIFYNLIKPYLIPQMMYKTPNTISSETFLK

SEQ ID NO: 57 - protein, 238 aa, Zygosaccharomyces bisporus
MKFIKKEQIKNLGPNSKLLKQYKSQLTNLTSEQLEIGVGLLLGDAYIRSRDNGKTNCIQFEWKNKAYIDHICLKFDEWVLSPPHKKMRINHLGNEVITWGAQTFKHEAFNELSKLFIINNKKHIINNLIEDYVTPKSLAYWFMDDGGKWDYNKGSMNKSIVLNTQCFTIDEVNSLINGLNTKFKLNCSMKFNKNKPIIYIPHNSYNIYYELISPYIITEMRYKLPSYEGTSKDYNKIH

SEQ ID NO: 58 - protein, 228 aa, Lachancea thermotolerans
MTMKYITKQQIKNLGPNSKLLKQYKAQLTRLTTVQLEAGVGLILGDAYIRSRDEGKTYCMQFEWKNEAYINHVCKLYDEWVLSSPHKKVRTNHLGNEVVTWGAQTFKHKAFNELAELFIINNNKHINPDLVNQYITPRSLAYWFMDDGGKWDYNTNSNNKSIVLNTQGFSIQEVQYLIDGLNIKFNLNCIMKFNKNKPIIFIPSDNYKHYYDLIIPYIIPEMKYKLPT

SEQ ID NO: 59 - protein, 230 aa, Pichia canadensis
MKKQIINKKDLLGLGPNSKLIKDYKKQWTTLSKIQEETLIGNILGDVYIKKLKRNKHFLLQFEWKNKAYIEHIVRVFDEYVISPPTLYERKNHLGNKVITWRAQTFEHKAFDKLGYYFMENHKKIIKPDLVLNYITERSLAYWFMDDGGKWDYNKKTKNKSLVLHTQGFKKEEVEILINDLNIKFNLNCSIKFNKNKPIIYIPNKDYELFYNLVNPYIIPEMKYKLLFNV

SEQ ID NO: 60 - protein, 163 aa, Chlamydomonas reinhardtii
MNTKYNKEFLLYLAGFVDGDGSIIAQIKPNQSYKFKHQLSLAFQVTQKTQRRWFLDKLVDEIGVGYVRDRGSVSDYILSEIKPLHNFLTQLQPFLKLKQKQANLVLKIIWRLPSAKESPDKFLEVCTWVDQIAALNDSKTRKTTSETVRAVLDSLSEKKKSSP

SEQ ID NO: 61 - protein, 171 aa, Carteria lunzensis
MNKFTPDQLLYLAGLIDGDGSIIAQLVSRKDYTWEFQIRLTVQVTQLKKRRWFLEELQKEIGAGSVRDRDTVSDYILTETSNVYKFLKDLQPHLRLKQKQANLVLRIIEQLPSSKASKEIFLELCNVVDHVATLNDTKKRKYTAEIVAAKLKELKECVVPVETSEETNSGI

SEQ ID NO: 62 - protein, 182 aa, Carteria olivieri
MKDLQEKDLIYLAGFIDADGSIFAQLISNNDYKFNYQIRVTVQITQLTKRKLFLTHIRDLIGVGTIRDRKNVSDYVLVEPRFVYKLLTQLQPFLRLKKKQANLVLKIIEQLPSSKDSQPEFLKLCKQVDQIAALNDSKKRKHTTSSVVMSLGHELPEKVSKEVNVPVETSDLIEIEEDPSSI

SEQ ID NO: 63 - protein, 167 aa, Scenedesmus obliquus
MTNNNMQNKGMKIIDKDELIYLAGFIDGDGSLIAQMVRRHDYKFKYQIKCTVQITQLKKRRHFLEKIQESIGYGIIRDRGTISDYVLVEPKCVYWLLKQLSPFLRLKKKQADLIIRIIEQLTSSKNSAVLFVQLCRLTDQVALLNDSKSRTITAEVVETTLRELGLI

SEQ ID NO: 64 - protein, 166 aa, Haematococcus lacustris
MKNINSTRFSHLTNEQKAYLAGFIDCDGSLMAQIVRKPDYAYKFQIRVTIQLSQRTSRIHFLKEIASEVGYGYVVSRNNMSDYVITQANIVYELLSLLLPYLRMKVKQANLILKIIQELPSAKVSKDKFIELCILANQVSILNTPNKILKNTWQVVKAELESEDLQ

SEQ ID NO: 65 - protein, 218 aa, Chlamydomonas moewusii
MSNFILKPGEKLPQDKLEELKKINDAVKKTKNFSKYLIDLRKLFQIDEVQVTSESKLFLAGFLEGEASLNISTKKLATSKFGLVVDPEFNVTQHVNGVKVLYLALEVFKTGRIRHKSGSNATLVLTIDNRQSLEEKVIPFYEQYVVAFSSPEKVKRVANFKALLELFNNDAHQDLEQLVNKILPIWDQMRKQQGQSNEGFPNLEAAQDFARNYKKGIK

SEQ ID NO: 66 - protein, 213 aa, Chlorococcum echinozygotum
MQNYTLKPGEVLPANVSQQLAKINNDFSKSSDFAKYLSNLRQLFQIEPIQVTSEAKLYLAGFVEGEGSLNISAKKTRHARFGVVVDPEFSITQHVNGFKPVYLALEVFKTGRIRHKGGSNATMVLTIDNRKSLEEKVIPFYEQYVAGFSSSSKNNRVTKFKTLLDLFNKGSHKDKDLLINDILPIWDELRQQKGQKNQAFKDLNEAATYIRQK

SEQ ID NO: 67 - protein, 229 aa, Chlorogonium elongatum
MNSSSENRKFFFLNPGEKLPEDIVTKLKQINDSFSKHSDFSRYKREIKELFQIAHIFVTEDSKRFLGGFLEGEASLNVSAKKLTNAKFGLLIDPEFSITQHVNGISNLYLALEVFQTGRISLKNGSNATMVFKIDNRQNLQQKVVPFYETYVNRYGSPNKKARVVLFLQLLDLFNQKGHENLQVFVEKMLPIWDKMRMQKGQSNEAFPDLDTAQRYVKNFWHNKNNNLT

SEQ ID NO: 68 - protein, 244 aa, Ankistrodesmus stipitatus
MQIKPLDITIVQSGIFLKPGEKISQEILEKLRKINKKYSETKNFPEYERSVRELFKLKPVQIKEKTMQFLAGFCEGEASMSAGAKKNSTSHFKVYIDPEFNLTQHVNGISNLYLALVSFKTGRIRHKISSNATFVFTIDNRQNLKEKVLPFYEKYVHPFGSPVKVRRTEMLKKLLSLFDEKAHLNSDRMINEVLPLWDAMRIQVGQSNETFQSLGEAQDYIRNAVRPLSSQGLVLKHKQKGDGN

SEQ ID NO: 69 - protein, 216 aa, Chlamydomonas monadina
MIIKSGEKIPDNILKELQGINEKYTKDRDFNIYLDRLGKLFNISKQNIRTEKKLFLAGYLEGEGSLSFSIKKNLNAKYGVTLDPEFNVTQHINGVEQLYTYLQIFQTGRITYKSGSNATLVFKISNRRSLEEKVIPFWNMYVAPYATQAKQQRFLKFQKVLELFREDCHTKLDCLTCEMLPLWDSMRIQKGQSNESFPDLQSAVDYVQTFVKKSKK

SEQ ID NO: 70 - protein, 218 aa, Chlamydomonas humicola
MSLTQQQKDLIFGSLLGDGNLQTGSVGRTWRYRALHKSEHQTYLFHKYEILKPLCGENTLPTESIVFDERTNKEVKRWFFNTLTNPSLKFFADMFYTYDQNTQKWVKDVPVKVQTFLTPQALAYFYIDDGALKWLNKSNAMQICTESFSQGGTIRIQKALKTLYNIDTTLTKKTLQDGRIGYRIAIPEASSGAFREVIKPFLVDCMRYKVSDGNKGHL

SEQ ID NO: 71 - protein, 218 aa, Chlamydomonas zebra; identical to SEQ ID NO: 70

SEQ ID NO: 72 - protein, 222 aa, Chlamydomonas monadina
MLTQHSFAMLEQRNLIFGSLLGDGNLQTGSNGRTWRYRAIQQQKHQAYLFHKYQILSPLCNTPPAFSQTFDPRTNNTSSRYTFNTLVSPCLDLYAQMFYTYDPSQGTWKKDVPSDIYLDKHLTPEAIAYWYMDDGALKWFNKSNAMRICTESFSLGGVMRLKRVLLERYNIVTRLNVKKLQNSISYRIAIPEISSEAFRDLIRPYLVDCMRYKVSDGYRGHL

SEQ ID NO: 73 - protein, 242 aa, Ankistrodesmus stipitatus
MTLTQHQKELLVGTLLGDGNLQTETRGRTWRYRAIQKAEHKDYLFHKYEILKEFCSTEPQLSRVADVRTNKTYERWMFSTKVHDSLRFYGNLFYTYDNKTQRMVKDIPVNIEKFLTPATVAYWYMDDGSLKYPGKSNALRICTESFSDDGVRRLQRALKNLYGIEASQTQKNKIVNGNKLPVGLRIAINERASTAFRELIEPYLVDCMKYKVSDGKKGRLLVLKQANSSENISAENSIHTEG

SEQ ID NO: 74 - protein, 222 aa, Neochloris aquatica
MTTFDQLSWNQQQVDLIVGTLLGDGNLSSESLTAGWRYRAAQKTEHLQYLEHKYQILKDSCGTSINNGDYYDPRTNKIYKRHYFNTLVHPDFKFFGEMFYTWDPILQKYKKDVPVDIHKYLTPAAIAYFYMDDGALKWKGQSNAMRICTESFSEEGVKRLQAAFWCHYKIYVSLTPHKKNGQFVGYRLFINEENSSRFRTLVAPHLVHCMRYKVSDGNYGTL

SEQ ID NO: 75 - protein, 194 aa, Desulfurococcus mobilis
MHNNENVSGISAYLLGLIIGDGGLYKLKYKGNRSEYRVVITQKSENLIKQHIAPLMQFLIDELNVKSKIQIVKGDTRYELRVSSKKLYYYFANMLERIRLFNMREQIAFIKGLYVAEGDKTLKRLRIWNKNKALLEIVSRWLNNLGVRNTIHLDDHRHGVYVLNISLRDRIKFVHTILSSHLNPLPPERAGGYT

SEQ ID NO: 76 - protein, 234 aa, Thermoproteus sp.
  misc_feature (185)..(185): Xaa can be any naturally occurring amino acid
MSVAYLLGLIVGDGGLYALRYRGGRTEYRVVITQKDERVVEKAVVMLEALLRELGLKSRVQVIRGRSRTEVRVSSKALWQFFNNVLSNLEGFQPSERAAFIEGLYDAEGDKSGRRARIWNKNLRLLELVKNWLSEFGIESTIHLDDKRHGVYVLEVPSPYRDRFFKLIHPPQPPDSSGVHEWINXVPTVPARGPANPPPGAQSWDPRRGEKSLWSFTAACRCGGAGDAERRQEQ

SEQ ID NO: 77 - protein, 234 aa, Thermoproteus sp.
MSVAYLLGLIVGDGGLYALRYRGGRTEYRVVITQKDEGVVEKAVVMLEALLRELGLKSRVQVIRGRSRTEVRVSSKALWQFFNNVLSNLEGFQPSERAAFIEGLYDAEGDKSGRRARIWNKNLQLLELVKNWLSEFGIESTIYLDDKRHGVYVLEVPSPYRDRFFKLIHPPQPPDSSGVHEWINEVPTVPARGPANPPPGAQSWDPRRGEKSLWSFTAACRCGGAGDAERRQEQ

SEQ ID NO: 78 - protein, 235 aa, Pyrobaculum calidifontis
MSSVAYLLGLIVGDGGLYLLRYKGGRTEYRVVVTQKDAAIAENAAKMFHSLLKELGLGSKVQVISGRTRVEVRVSSKRLWQLFNDKLANLEGLAPDERIAFIRGLYDAEGDKTGRRARLWNKNRRLLDLVGSWLRELGIESRVYLDDKRHGVYVLEVPSPYRRRFFELLYPPQPPDSSGVHEWINEVPTVPARGPANPPPGAQSWDPRRGEKSLWSFTAACRCGGAGGAERRWER

SEQ ID NO: 79 - protein, 1071 aa, Saccharomyces cerevisiae
MAGAIENARKEIKRISLEDHAESEYGAIYSVSGPVVIAENMIGCAMYELVKVGHDNLVGEVIRIDGDKATIQVYEETAGLTVGDPVLRTGKPLSVELGPGLMETIYDGIQRPLKAIKEESQSIYIPRGIDTPALDRTIKWQFTPGKFQVGDHISGGDIYGSVFENSLISSHKILLPPRSRGTITWIAPAGEYTLDEKILEVEFDGKKSDFTLYHTWPVRVPRPVTEKLSADYPLLTGQRVLDALFPCVQGGTTCIPGAFGCGKTVISQSLSKYSNSDAIIYVGCFAKGTNVLMADGSIECIENIEVGNKVMGKDGRPREVIKLPRGRETMYSVVQKSQHRAHKSDSSREVPELLKFTCNATHELVVRTPRSVRRLSRTIKGVEYFEVITFEMGQKKAPDGRIVELVKEVSKSYPISEGPERANELVESYRKASNKAYFEWTIEARDLSLLGSHVRKATYQTYAPILYENDHFFDYMQKSKFHLTIEGPKVLAYLLGLWIGDGLSDRATFSVDSRDTSLMERVTEYAEKLNLCAEYKDRKEPQVAKTVNLYSKVVRGNGIRNNLNTENPLWDAIVGLGFLKDGVKNIPSFLSTDNIGTRETFLAGLIDSDGYVTDEHGIKATIKTIHTSVRDGLVSLARSLGLVVSVNAEPAKVDMNGTKHKISYAIYMSGGDVLLNVLSKCAGSKKFRPAPAAAFARECRGFYFELQELKEDDYYGITLSDDSDHQFLLANQVVVHNCGERGNEMAEVLMEFPELYTEMSGTKEPIMKRTTLVANTSNMPVAAREASIYTGITLAEYFRDQGKNVSMIADSSSRWAEALREISGRLGEMPADQGFPAYLGAKLASFYERAGKAVALGSPDRTGSVSIVAAVSPAGGDFSDPVTTATLGITQVFWGLDKKLAQRKHFPSINTSVSYSKYTNVLNKFYDSNYPEFPVLRDRMKEILSNAEELEQVVQLVGKSALSDSDKITLDVATLIKEDFLQQNGYSTYDAFCPIWKTFDMMRAFISYHDEAQKAVANGANWSKLADSTGDVKHAVSSSKFFEPSRGEKEVHGEFEKLLSTMQERFAESTD

SEQ ID NO: 80 - protein, 1022 aa, Saccharomyces cerevisiae
  misc_feature (318)..(318): Xaa can be any naturally occurring amino acid
GAIYSVSGPVVIAENMIGCAMYELVKVGHDNLVGEVIRIDGDKATIQVYEETAGLTVGDPVLRTGKPLSVELGPGLMETIYDGIQRPLKAIKEESQSIYIPRGIDTPALDRTIKWQFTPGKFQVGDHISGGDIYGSVFENSLISSHKILLPPRSRGTITWIAPAGEYTLDEKILEVEFDGKKSDFTLYHTWPVRVPRPVTEKLSADYPLLTGQRVLDALFPCVQGGTTCIPGAFGCGKTVISQSLSKYSNSDAIIYVGCFAKGTNVLMADGSIECIENIEVGNKVMGKDGRPREVIKLPRGRETMYSVVQKSQHRAHXSDSSREVPELLKFTCNATHELVVRTPRSVRRLSRTIKGVEYFEVITFEMGQKKAPDGRIVELVKEVSKSYPISEGPERANELVESYRKASNKAYFEWTIEARDLSLLGSHVRKATYQTYAPILYENDHFFDYMQKSKFHLTIEGPKVLAYLLGLWIGDGLSDRATFSVDSRDTSLMERVTEYAEKLNLCAEYKDRKEPQVAKTVNLYSKVVRGNGIRNNLNTENPLWDAIVGLGFLKDGVKNIPSFLSTDNIGTRETFLAGLIDSDGYVTDEHGIKATIKTIHTSVRDGLVSLARSLGLVVSVNAEPAKVDVNGTKHKISYAIYMSGGDVLLNVLSKCAGSKKFRPAPAAAFARECRGFYFELQELKEDDYYGITLSDDSDHQFLLANQVVVHNCGERGNEMAEVLMEFPELYTEMSGTKEPIMKRTTLVANTSNMPVAAREASIYTGITLAEYFRDQGKNVSMIADSSSRWAEALREISGRLGEMPADQGFPAYLGAKLASFYERAGKAVALGSPDRTGSVSIVAAVSPAGGDFSDPVTTATLGITQVFWGLDKKLAQRKHFPSINTSVSYSKYTNVLNKFYDSNYPEFPVLRDRMKEILSNAEELEQVVQLVGKSALSDSDKITLDVATLIKEDFLQQNGYSTYDAFCPIWKTFDMMRAFISYHDEAQKAVANGANWSKLADSTGDVKHAVSSSKFFEPSRG

SEQ ID NO: 81 - protein, 1045 aa, Saccharomyces pastorianus
ISLEDHAESEYGAIYSVSGPVVIAENMIGCAMYELVKVGHDNLVGEVIRIDGDKATIQVYEETAGLTVGDPVLRTGKPLSVELGPGLMETIYDGIQRPLKAIKEESQSIYIPRGIDTPALDRTIKWQFTPGKFQVGDHISGGDIYGSVFENSLISSHKILLPPRSRGTITWIAPAGEYTLDEKILEVEFDGKKSDFTLYHTWPGRVPRPVTEKLSADYPLLTGQRVLDALFPCVQGGTTCIPGAFGCGKTVISQSLSKYSNSDAIIYVGCFAKGTNVLMADGSIECIENIEVGNKVMGKDGRPREVIKLPRGSETMYSVVQKSQHRAHKSDSSREMPELLKFTCNATHELVVRTPRSVRRLSRTIKGVEYFEVITFEMGQKKAPDGRIVELVKEVSKSYPVSEGPERANELVESYRKASNKAYFEWTIEARDLSLLGSHVRKATYQTYAPILYENDHFFDYMQKSKFHLTIEGPKVLAYLLGLWIGDGLSDRATFSVDSRDTSLMERVTEYAEKLNLCAEYKDRKEPQVAKTVNLYSKVVRGNGVRNNLNTENPLWDAIIGLGFLKDGVKNIPSFLSTDNIGTRETFLAGLIDSDGYVTDEHGIKATIKTIHTSVRDGLVSLARSLGLVASVNAEPAKVDMNGTKHKISYAIYMSGGDVLLNVLSKCAGSKKFRPAPVATFVRECQGFYFELQELKENDYYGITLSDDSDHQFLLANQVVVHNCGERGNEMAEVLMEFPELYTEMSGTKEPIMKRTTLVANTSNMPVAAREASIYTGITLAEYFRDQGKNVSMIADSSSRWAEALREISGRLGEMPADQGFPAYLGAKLASFYERAGKAVALGSPDRTGSVSIVAAVSPAGGDFSDPVTTATLGITQVFWGLDKKLAQRKHFPSINTSVSYSKYTNVLNKFYDSNYPEFPVLRDRMKEILSNAEELEQVVQLVGKSALSDDKITLDVATLIKEDFLQQNGYSTYDAFCPIWKTFDMMRAFISYHDEAQKAVANGANWSKLADSTGDVKHAVSSSKFFEPSRGEKEVHGEFEKLLS

SEQ ID NO: 82 - protein, 545 aa, Saccharomyces cariocanus
  misc_feature (291)..(291): Xaa can be any naturally occurring amino acid
ISQSLSKYSNSDAIIYVGCFAKGTTVLMADGSIECIENIKIGDKVMGKDGKPREVIKLPRGNETMYSVVQKSQHRAHKTDSSREVPDLLKFTCNSTHELVVRTPRSVRRVSRTMKGVEYFEVISFEMVQKKVPDGRIIELVKEVSKSYPASEGPERADELVESYRKASTKPYFEWTVEARDLSLLGSHVRKATYQTYAPILYENDYFFNYMENSKFHPTIEAPKVLAYFLGLWIGDGLTDRTTFSIDSRDTSLMERVTEYAEKLDLCAEYKDRKEPKVAKTVNLYSKSVRXNGIRNNLNTENPLWDAIVGLGFLKDGVKNIPSFLSTDNIGTRETFLAGLIDSDGYVTDEHGITATVKTIHTSVRDGLVSVARSLGLVISVNAEPAKIDMSGTSHKMCYAIYMSGGDILLNVLSKCASFKKFRPAPVAPPVRECRGFYFELQELEEDDYYGITLSDDSDHQFLLANQVVVHNCGERGNEMAEVLMEFPELYTEMSGTKEPIMKRTTLVANTSNMPVAAREASIYTGITLAEYFRDQGKNVSMIAD

SEQ ID NO: 83 - protein, 547 aa, Zygosaccharomyces bailii
  misc_feature (185)..(185): Xaa can be any naturally occurring amino acid
ISQSLSKYSNSDAIIYVGCFAKGTEVMMHDGSVKAIETIEAGEAVMGTDGQPRKVVGLPRGREVMYKVSQKTAHRVHKTDETRAAPVALFEYNCNATHKLVVRTPRSCRSITRKMQGVDYNEVIFFDLKKKKLEDGREIEIVKEVSRSYPAAEGAEKAAQMVKDYYDAARGKEFFEWTIEARDVXELGAHVRKATHQVYAPVLYESDFFFHYVKNSKFALRSEASTALAYLLGLWVGDGLSDRAVLSVDSEDSSLLERITGYADILDLSAEYKDREIPKRAKTVCLYPKTIRGNDIRRNLNTDNPVWNAIVDLGYLKDGVKNVPSYLFSDSICHREVFLAGLIDSDGHVRGDDGLSVTIKTIHKTVMEGTVAVARSLGLIVSVNTEEAKIDKNDVNHRFVYAIYISGGDALLSVLAHCAAAKKFRAPPSNEVVRGLKKVFFEMEELKEDDYYGITLAKESDHQFLLANQLVVHNCGERGNEMAEVLMEFPELFTEKNGRKEPIMKRTTLVANTSNMPVAAREASIYTGITLAEYFRDQGKNISMIAD

SEQ ID NO: 84 - protein, 170 aa, Monomastix spec.
MTTKNTLQPTEAAYIAGFLDGDGSIYAKLIPRPDYKDIKYQVSLAISFIQRKDKFPYLQDIYDQLGKRGNLRKDRGDGIADYTIIGSTHLSIILPDLVPYLRIKKKQANRILHIINLYPQAQKNPSKFLDLVKIVDDVQNLNKRADELKSTNYDRLLEEFLKAGKIESSP

SEQ ID NO: 85 - protein, 167 aa, Monomastix spec.
MKTLESTLAAYIAGFLDGDGSIYAKVISRPDYAVIKYQISVSLSFCQRKDRYTYLQDIYEALDKCGSLRKDRGDGIADYTITGPAHLSIVLPHLLPYLRIKKKQANLVLHIINQYPAAKKNHLEFLSLVKLVDQIQNLNKKPDEPKATNYQSLLEEFQTAGRIQSSP

SEQ ID NO: 86 - protein, 180 aa, Escherichia coli
MTKLQPNTVIRAALDLLNEVGVDGLTTRKLAERLGVQQPALYWHFRNKRALLDALAEAMLAENHTHSVPRADDDWRSFLIGNARSFRQALLAYRDGARIHAGTRPGAPQMETADAQLRFLCEAGFSAGDAVNALMTISYFTVGAVLEEQAGDSDAGERGGTVEQAPLSPLLRAAIDAFDE

SEQ ID NO: 87 - protein, 208 aa, Klebsiella pneumoniae
MTKLQPNTVIRAALDLLNEVGVDGLTTRKLAERLGVQQPALYWHFRNKRALLDALAEAMLAENHTHSVPRADDDWRSFLIGNARSFRQALLAYRDGARIHAGTRPGAPQMETADAQLRFLCEAGFSAGDAVNALMTISYFTVGAVLEEQAGGTVEQAPLSPLLRAAIDAFDEAGPDAAFEQGLAVIVDGLAKRRLVVRNVEGPRKGDD

SEQ ID NO: 88 - protein, 213 aa, Klebsiella pneumoniae
MIKLQPNTVIRVALDLLNEVGVEALTTRKLAKRLGVQQPALYWHFRNKRALLDALAEAMLAENHTHSVPRVDDDWRSFLIGNARSFRQALLAYRDGARIHAGTRPGAPQMEVVDAQLRFLCEAGFSAWDAVNALMTISYFTVGAVLEEQAGDSDAGERGGTIEQAPLLRAVIDTFDEAGPDAVFELGLAVIVDGLAKRRLVARNIQGPRKGDD

SEQ ID NO: 89 - protein, 203 aa, Laribacter hongkongensis
MTKLQPNTVIRAALDLLNEVGVDGLTTRKLAERLGVQQPALYWHFRNKRALLDALAEAMLAENHTHSVPRADDDWRSFLKGNACSFRRALLAYRDGARIHAGTRPAAPQMEKADAQLRFLCDAGFSAGDATYALMAISYFTVGAVLEQQASEADAEERGEDQLTTSASTMPARLQSAMKIVYEGGPDAAFERGLALIIGGLER

SEQ ID NO: 90 - protein, 206 aa, Aeromonas salmonicida
MKKLQREAVIRTALELLNDVGMEGLTTRRLAERLGVQQPALYWHFRNKRALLDALAEAMLTINHTHSTPRDEDDWRSFLKGNACSFRRALLAYRDGARIHAGTRPAAPQMEKADAQLRFLCDAGFLAGDATYALMAISYFTVGAVLEQQASEADAEERGEDQLTTSASTMPARLQSAMKIVYEGGPDAAFERGLALIIGGLEQVRL

SEQ ID NO: 91 - protein, 61 aa, Escherichia coli
KLQPNTVIRAALDLLNEVGVDGLTTRKLAERLGVQQPALYWHFRNKRALLDALAEAMLAEN

SEQ ID NO: 92 - protein, 61 aa, Klebsiella pneumoniae
KLQPNTVIRVALDLLNEVGVEALTTRKLAKRLGVQQPALYWHFRNKRALLDALAEAMLAEN

SEQ ID NO: 93 - protein, 60 aa, Acinetobacter baumannii
KLDKGTVIAAALELLNEVGMDSLTTRKLAERLKVQQPALYWHFQNKRALLDALAEAMLAE

SEQ ID NO: 94 - protein, 61 aa, Aeromonas salmonicida
KLQREAVIRTALELLNDVGMEGLTTRRLAERLGVQQPALYWHFRNKRALLDALAEAMLTIN

SEQ ID NO: 95 - protein, 60 aa, Ochrobactrum anthropi
KLHRDAVIQTALELLNEVGEEGLTTRRLAERLGVQQPALYWHFKNKRVLLDALAETILAE

SEQ ID NO: 96 - protein, 74 aa, Staphylococcus aureus
KFAKDRIIKLICHLCQTVGYDQDEFYEIKQFLTIQLMSDMAGISRETAGHIIHELKDEKLVVKDHKNWLVSKHL

SEQ ID NO: 97 - protein, 74 aa, Staphylococcus aureus
KLAKERVTKILRYLCQTVGYDHDEFYEIKHFMTIQLLSDMAGISRETTSHIINELKEEKILFKNSKNWLVSKDL

SEQ ID NO: 98 - protein, 74 aa, Staphylococcus epidermidis
KLAKERVTKILRYLCQTVGYDHDEFYEIKHFMTIQLLSDMAGISRETTSHIINELREEKILFKNSKNWLVSKDL

SEQ ID NO: 99 - protein, 74 aa, Staphylococcus epidermidis
KLAKERVTKILRYLCHTVGYDNEEFYEIKQFMTIQLLSDMAGISRETTGHIINELREDKVLFKSNKNWLISKEL

SEQ ID NO: 100 - protein, 72 aa, Staphylococcus haemolyticus
KLARERIEKVLYYLCHAIGYDQDEFYEIKHIMTIQLLSDLAGISRETTGHIVHELKEEKKLIKNGKNWMVIK

SEQ ID NO: 101 - protein, 58 aa, Escherichia coli
MKPVTLYDVAEYAGVSYQTVSRVVNQASHVSAKTREKVEAAMAELNYIPNRVAQQLAG

SEQ ID NO: 102 - protein, 58 aa, Klebsiella pneumoniae
VKPVTLYDVAEYAGVSYQTVSRVVNQASHVSAKTREKVEAAMAQLNYIPNRVAQQLAG

SEQ ID NO: 103 - protein, 58 aa, Citrobacter koseri
VKPVTLYDVADRAGVSYQTVSRVVNQASHVSAKTREKVEAAMAELNYIPNRVAQQLAG

SEQ ID NO: 104 - protein, 57 aa, Erwinia carotovora
KPITLHDVAEYAGVSYQTVSRVLNQAPHVSSRTRNKVEQAMAALNYTPNRVAQQLAG

SEQ ID NO: 105 - protein, 58 aa, Enterobacter cancerogenus
MKAITLYDVARLAGVSYQTVSRVINEAEHVSARTREKVLRAMAELHYVPNRGAQQLAG

SEQ ID NO: 106 - protein, 80 aa, Clostridium butyricum
MKFRIGELADKCGVNKETIRYYERLGLIPEPERTEKGYRMYSQQTVDRLHFIKRMQELGFTLNEIDKLLGVVDRDEAKCR

SEQ ID NO: 107 - protein, 80 aa, Bacillus macroides
MQFRIGELADKCGVNKETIRYYERLGLIPEPDRTEKGYRMYSKQTVDRLNFIKRMQELGFTLNEIDKLLGVVDRDEAKCR

SEQ ID NO: 108 - protein, 80 aa, Bacillus licheniformis
MQYRIGELAEKCSVNKETIRYYERLGLIPEPNRTEKGYRMYSLQTIDRLNFIKRMQELGFTLNEIDKLLGVVDRDEAKCR

SEQ ID NO: 109 - protein, 80 aa, Lysinibacillus fusiformis
MDFRVGEIAKKCNINKETIRYYERLGLIPEPDRTEKGYRMYSQQTVDRLNFIKRMQELGFTLNEIDKFLGVVDRDEAKCR

SEQ ID NO: 110 - protein, 80 aa, Bacillus cereus
MQFRIGELAEKCSVNKETIRYYERIGLIPEPDRTESGYRMYSQQIIDRLNFIKGMQELGFTLNEIDKLLGVVDRDESKCR

SEQ ID NO: 111 - protein, 78 aa, Geobacillus sp.
YRIGELAETCHVNKETIRYYERKGLIPETERTEGGYRLYTEETVRRIQFIKRLQGLGFTLAEIDKLLGVVDRDRDRCK

SEQ ID NO: 112 - protein, 100 aa, Escherichia coli
IHSILDWIEDNLESPLSLEKVSERSGYSKWHLQRMFKKETGHSLGQYIRSRKMTEIAQKLKESNEPILYLAERYGFESQQTLTRTFKNYFDVPPHKYRMT

SEQ ID NO: 113 - protein, 100 aa, Salmonella paratyphi
IHSILDWIEDNLESPLSLEKVSERSGYSKWHLQRMFKKETGHSLGQYIRSRKMTEIAQKLKESNEPILYLAERYGFESQQTLTRTFKNYFDVPPHKYRIT

SEQ ID NO: 114 - protein, 100 aa, Salmonella choleraesuis; identical to SEQ ID NO: 113

SEQ ID NO: 115 - protein, 100 aa, Enterobacter cancerogenus
IHSILDWIEDNLESPLSLEKVSERSGYSKWHLQRMFKKETGHSLGQYIRSRKLTEIAQKLKESNEPILYLAERYGFESQQTLTRTFKNYFDVPPHKYRIT

SEQ ID NO: 116 - protein, 100 aa, Cronobacter turicensis
IHSILDWIEDNLESPLSLEKVSARSGYSKWHLQRMFKKETGHSLGQYIRNRKLTEIALKLKESDEPILYLAERYGFESQQTLTRTFKNYFSVPPHKYRVT

SEQ ID NO: 117 - protein, 98 aa, Pantoea sp.
IRSLLDWIEDNLGHDLHLDEVARRSGYSRWHLQRLFRQHTGFSLAEYIRQRRLTESALTLLNSDEAILQVAMSYGFDTQQAYTRTFKNYFRVTPGQLR

SEQ ID NO: 118 - protein, 98 aa, Pantoea ananatis
IRSLLEWIESNLGHDLHLDEVARRAGYSRWHLQRLFRQHTGFSLAEYIRQRRLTESALTLLNSNEAILQVAMSYGFDTQQAYTRTFKNYFMVTPGQLR

SEQ ID NO: 119 - protein, 100 aa, Serratia proteamaculans
IHDLLDWIENHLDQPLLLDNVAAKSGYSKWHLQRMFRSTTGHALGSYIRERRLSQAAQALRSSPRPILDIALQFHFDSQPSFSRAFKKQFGKTPAVYRRT

SEQ ID NO: 120 - protein, 129 aa, Escherichia coli
MTMSRRNTDAITIHSILDWIEDNLESPLSLEKVSERSGYSKWHLQRMFKKETGHSLGQYIRSRKMTEIAQKLKESNEPILYLAERYGFESQQTLTRTFKNYFDVPPHKYRMTNMQGESRFLHPLNHYNN

SEQ ID NO: 121 - protein, 144 aa, Salmonella paratyphi
MSICSRKFCRRQKRGMTMSRRNTDAITIHSILDWIEDNLESPLSLEKVSERSGYSKWHLQRMFKKETGHSLGQYIRSRKMTEIAQKLKESNEPILYLAERYGFESQQTLTRTFKNYFDVPPHKYRITNMHGESRYMLPLNHGNY

SEQ ID NO: 122 - protein, 127 aa, Salmonella choleraesuis
MSRRNTDAITIHSILDWIEDNLESPLSLEKVSERSGYSKWHLQRMFKKETGHSLGQYIRSRKMTEIAQKLKESNEPILYLAERYGFESQQTLTRTFKNYFDVPPHKYRITNMHGESRYMLPLNHGNY

SEQ ID NO: 123 - protein, 128 aa, Enterobacter cancerogenus
MTMSRRNTDAITIHSILDWIEDNLESPLSLEKVSERSGYSKWHLQRMFKKETGHSLGQYIRSRKLTEIAQKLKESNEPILYLAERYGFESQQTLTRTFKNYFDVPPHKYRITSMPGESRYLYPLKHCS

SEQ ID NO: 124 - protein, 124 aa, Cronobacter turicensis
MSRRNNDAITIHSILDWIEDNLESPLSLEKVSARSGYSKWHLQRMFKKETGHSLGQYIRNRKLTEIALKLKESDEPILYLAERYGFESQQTLTRTFKNYFSVPPHKYRVTRMPGEGKYLHPLNH

SEQ ID NO: 125 - protein, 122 aa, Pantoea sp.
MNQSQFIRSLLDWIEDNLGHDLHLDEVARRSGYSRWHLQRLFRQHTGFSLAEYIRQRRLTESALTLLNSDEAILQVAMSYGFDTQQAYTRTFKNYFRVTPGQLRRQRRVEPDRLLFPLAVAS

SEQ ID NO: 126 - protein, 135 aa, Pantoea ananatis
MCSLHICFNQENTMNQREFIRSLLEWIESNLGHDLHLDEVARRAGYSRWHLQRLFRQHTGFSLAEYIRQRRLTESALTLLNSNEAILQVAMSYGFDTQQAYTRTFKNYFMVTPGQLRRQRRVEPDRLLFPYAMAS

SEQ ID NO: 127 - protein, 152 aa, Serratia proteamaculans
MDRVNIIHDLLDWIENHLDQPLLLDNVAAKSGYSKWHLQRMFRSTTGHALGSYIRERRLSQAAQALRSSPRPILDIALQFHFDSQPSFSRAFKKQFGKTPAVYRRTTRWDVAEMRPQAIEPLNGHRHESPGVHLWYAGKSLDGVCTNWVGQH

SEQ ID NO: 128 - protein, 34 aa, Artificial Sequence
  TAL repeat 34
LTPEQVVAIASNGGGKQALETVQRLLPVLCQAHG

SEQ ID NO: 129 - protein, 35 aa, Artificial Sequence
  TAL repeat 35
LTPEQVVAIASNGGGKQALETVQRLLPVLCQAPHD

SEQ ID NO: 130 - DNA, 13 bp, Artificial Sequence
  scTet recognition sequence
ytatcattgatag

SEQ ID NO: 131 - DNA, 18 bp, Artificial Sequence
  DNA recogntion sequence of TraR
atgtgcagatctgcacat

SEQ ID NO: 132 - DNA, 20 bp, Artificial Sequence
  DNA recognition site variant of LacR
tgtttgatatcatataaaca

SEQ ID NO: 133 - DNA, 23 bp, Artificial Sequence
  LacR recogntion site variant 2
gaattgtgagcggataacaattt

SEQ ID NO: 134 - DNA, 22 bp, Artificial Sequence
  LacR recognition site variant 3
gaatgtgagcgagtaacaaccg

SEQ ID NO: 135 - DNA, 22 bp, Artificial Sequence
  LacR recognition site variant 4
cggcagtgagcgcaacgcaatt

SEQ ID NO: 136 - DNA, 21 bp, Artificial Sequence
  LacR recogntion site variant 5
gaattgtaagcgcttacaatt

SEQ ID NO: 137 - DNA, 20 bp, Artificial Sequence
  MarR recognition site
ayngcacnnwnnryyaaayn

SEQ ID NO: 138 - DNA, 31 bp, Artificial Sequence
  MerR recognitition site
ttkacynnnnnnnnnnnnnnnnnnntaaggt

SEQ ID NO: 139 - DNA, 22 bp, Artificial Sequence
  I-CreI cutting or recognition site
caaaacgtcgtgagacagtttc

SEQ ID NO: 140 - DNA, 22 bp, Artificial Sequence
  I-CeuI cuttin or recognition site
ataacggtcctaaggtagcgaa

SEQ ID NO: 141 - DNA, 30 bp, Artificial Sequence
  I-DmoI cutting or recognition site
atgccttgccgggtaagttccggcgcgcat

SEQ ID NO: 142 - DNA, 31 bp, Artificial Sequence
  DNA recognition site of I-AniI
gcgcgctgaggaggtttctctgtaaagcgca

SEQ ID NO: 143 - protein, 254 aa, Artificial Sequence
  endonuclease fragment of Aspergillus nidulans protein
GSDLTYAYLVGLFEGDGYFSITKKGKYLTYELGIELSIKDVQLIYKIKKILGIGIVSFRKINEIEMVALRIRDKNHLKSFILPIFEKYPMFSNKQYDYLRFRNALLSGIISLEDLPDYTRSDEPLNSIESIINTSYFSAWLVGFIEAEGCFSVYKLNKDDDYLIASFDIAQRDGDILISAIRKYLSFTTKVYLDKTNCSKLKVTSVRSVENIIKFLQNAPVKLLGNKKLQYLLWLKQLRKISRYSEKIKIPSNY

SEQ ID NO: 144 - protein, 15 aa, Artificial Sequence
  sequence motif of I-SceI
HVCLLYDQWVLSPPH

SEQ ID NO: 145 - protein, 11 aa, Artificial Sequence
  sequence motif of I-SceI
LAYWFMDDGGK

SEQ ID NO: 146 - protein, 27 aa, Artificial Sequence
  sequence motif of I-SceI
KTIPNNLVENYLTPMSLAYWFMDDGGK

SEQ ID NO: 147 - protein, 19 aa, Artificial Sequence
  sequence motif of I-SceI
KPIIYIDSMSYLIFYNLIK

SEQ ID NO: 148 - protein, 19 aa, Artificial Sequence
  sequence motif of I-SceI
KPIIYIDSMSYLIFYNLIK

SEQ ID NO: 149 - protein, 9 aa, Artificial Sequence
  alternative C-terminal sequence of I-SceI
TIKSETFLK

SEQ ID NO: 150 - protein, 9 aa, Artificial Sequence
  optimized C-terminus of I-SceI
AIANQAFLK

SEQ ID NO: 151 - protein, 27 aa, Artificial Sequence
  amino acid linker sequence comprising a nuclear localisation signal
RSGGGSGGGTGGGSGGGAPKKKRKVLE

SEQ ID NO: 152 - protein, 12 aa, Artificial Sequence
  short amino acid linker
RSAPKKKRKVLE

SEQ ID NO: 153 - DNA, 22 bp, Artificial Sequence
  DNA recognition site of I-MsoI
cagaacgtcgtgagacagttcc

SEQ ID NO: 154 - DNA, 30 bp, Artificial Sequence
  DNA recognition site of Pi-SceI
atctatgtcgggtgcggagaaagaggtaat

SEQ ID NO: 155 - protein, 6 aa, Artificial Sequence
  amino acid linker sequence 1
GSGSGS

SEQ ID NO: 156 - protein, 5 aa, Artificial Sequence
  amino acid linker sequence 2
GGSGG

SEQ ID NO: 157 - protein, 8 aa, Artificial Sequence
  amino acid linker 3
GGSGGSGG

SEQ ID NO: 158 - protein, 8 aa, Artificial Sequence
  amino acid linker 4
GSGSGGSG

SEQ ID NO: 159 - protein, 238 aa, Artificial Sequence
  I-SceI comprising a deletion of 5 amino acids at the C-terminus
MGPKKKRKVKNIKKNQVMNLGPNSKLLKEYKSQLIELNIEQFEAGIGLILGDAYIRSRDEGKTYCMQFEWKNKAYMDHVCLLYDQWVLSPPHKKERVNHLGNLVITWGAQTFKHQAFNKLANLFIVNNKKTIPNNLVENYLTPMSLAYWFMDDGGKWDYNKNSTNKSIVLNTQSFTFEEVEYLVKGLRNKFQLNCYVKINKNKPIIYIDSMSYLIFYNLIKPYLIPQMMYKLPNTISS

SEQ ID NO: 160 - protein, 1164 aa, Xanthomonas campestris pv. vesicatoria
  N-terminus (1)..(288)
  Repeat 1 (289)..(322)
  Repeat 2 (323)..(356)
  Repeat 3 (357)..(390)
  Repeat 4 (391)..(424)
  Repeat 5 (425)..(458)
  Repeat 6 (459)..(492)
  Repeat 7 (493)..(526)
  Repeat 8 (527)..(560)
  Repeat 9 (561)..(594)
  Repeat 10 (595)..(628)
  Repeat 11 (629)..(662)
  Repeat 12 (663)..(696)
  Repeat 13 (697)..(730)
  Repeat 14 (731)..(764)
  Repeat 15 (765)..(798)
  Repeat 16 (799)..(832)
  Repeat 17 (833)..(866)
  Repeat 17.5 (867)..(886)
  C-terminus (887)..(1164)
MDPIRSRTPSPARELLPGPQPDGVQPTADRGVSPPAGGPLDGLPARRTMSRTRLPSPPAPSPAFSAGSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPQQVVAIASNGGGKQALETVQRLLPVLCQAHGLTPQQVVAIASNSGGKQALETVQRLLPVLCQAHGLTPEQVVAIASNGGGKQALETVQRLLPVLCQAHGLTPEQVVAIASNIGGKQALETVQALLPVLCQAHGLTPEQVVAIASNIGGKQALETVQALLPVLCQAHGLTPEQVVAIASNIGGKQALETVQALLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASNSGGKQALETVQALLPVLCQAHGLTPEQVVAIASNSGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGRPALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRASSRKRSRSDRAVTGPSAQQSFEVRVPEQRDALHLPLSWRVKRPRTSIGGGLPDPGTPTAADLAASSTVMREQDEDPFAGAADDFPAFNEEELAWLMELLPQ

SEQ ID NO: 161 - protein, 1321 aa, Xanthomonas campestris pv. armoraciae
  N-terminus (1)..(288)
  Repeat 1 (289)..(323)
  Repeat 2 (324)..(358)
  Repeat 3 (359)..(393)
  Repeat 4 (394)..(428)
  Repeat 5 (429)..(463)
  Repeat 6 (464)..(498)
  Repeat 7 (499)..(533)
  Repeat 8 (534)..(568)
  Repeat 9 (569)..(603)
  Repeat 10 (604)..(638)
  Repeat 11 (639)..(673)
  Repeat 12 (674)..(708)
  Repeat 13 (709)..(743)
  Repeat 14 (744)..(778)
  Repeat 15 (779)..(813)
  Repeat 16 (814)..(848)
  Repeat 17 (849)..(883)
  Repeat 18 (884)..(918)
  Repeat 19 (919)..(953)
  Repeat 20 (954)..(988)
  Repeat 21 (989)..(1023)
  Repeat 21.5 (1024)..(1043)
  C-terminus (1044)..(1321)
MDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNNGGKQALETVQRLLPVLCQAPHDLTPEQVVAIASIGGGKQALETVQRLLPVLCQAPHDLTPEQVVAIASNGGGKQALETVQRLLPVLCQAPHCLTPEQVVAIASNIGGKQALETVQALLPVLCQAPHCLTPEQVVAIASNGGGKQALETVQRLLPVLCQAPHDLTPEQVVAIASNGGGKQALETVQRLLPVLCQAPHDLTPEQVVAIASHDGGKQALETVQRLLPVLCQAPHDLTPQQVVAIASNGGGKQALETVQRLLPVLCQAPHDLTPEQVVAIASHDGGKQALETVQRLLPVLCQAPHDLTPEQVVAIASNIGGKQALETVQALLPVLCQAPHCLTPEQVVAIASHDGGKQALETVQALLPVLCQAPHDLTPEQVVAIASNIGGKQALETVQRLLPVLCQAPHDLTREQVVAIASHDGGKQALETVQRLLPVLCQAPHDLTPEQVVAIASNGGGKQALETVQRLLPVLCQAPHDLTPEQVVAIASHDGGKQALETVQRLLPVLCQAPHDLTPEQVVAIASNGGGKQALETVQRLLPVLCQAPHDLTPEQVVAIASHDGGKQALETVQRLLPVLCQAPHDLTPEQVVAIASHDGGKQALETVQRLLPVLCQAPHDLTPEQVVAIASNGGGKQALETVQRLLPVLCQAPHDLTPEQVVAIASNGGGKQALETVQALLPVLCQAPHDLTPEQVVAIASNIGGKQALETVQRLLPVLCQAPHDLTPEQVVAIASNGGGKQALESIFAQLSRPDPALAALTNDRLVALACIGGRSALNAVKDGLPNALTLIRRANSRIPERTSHLVADHTQVVRVLGFFQCHSHPAQAFDEAMTQFGMSRHGLLQLFRRVGVTELEARSGTLPPASQRWDRILQASGMKRAKPSPTSTQTPDQASLHAFADSLERDLDAPSPMHEGDQTRASSRKRSRSDRAVTGPSAQQSFEVRVPEQRDALHLPLLSWGVKRPRTRIGGLLDPGTPMDADLVASSTVVWEQDADPFAGTADDFPAFNEEELAWLMELLPH

SEQ ID NO: 162 - protein, 960 aa, Xanthomonas campestris pv. armoraciae
  N-terminus (1)..(288)
  Repeat 1 (289)..(322)
  Repeat 2 (323)..(356)
  Repeat 3 (357)..(390)
  Repeat 4 (391)..(424)
  Repeat 5 (425)..(458)
  Repeat 6 (459)..(492)
  Repeat 7 (493)..(526)
  Repeat 8 (527)..(560)
  Repeat 9 (561)..(594)
  Repeat 10 (595)..(628)
  Repeat 11 (629)..(662)
  Repeat 11.5 (663)..(682)
  C-terminus (683)..(960)
MDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNIGGKQALETVQRLLPVLCQAHGLTPQQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASHDGGKQALETVQRLLPVLCQAHGLTPQQVVAIASHDGGKQALETVQRLLPVLCQAHGLTPQQVVAIASNSGGKQALETVQRLLPVLCQAHGLTPQQVVAIASNSGGKQALETVQRLLPVLCQAHGLTPQQVVAIASNSGGKQALETVQRLLPVLCQAHGLTPEQVVAIASHDGGKQALETVQRLLPVLCQAHGLTPEQVVAIASNIGGKQALETVQRLLPVLCQAHGLTPQQVVAIASNGGGRPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRASSRKRSRSDRAVTGPSAQQSFEVRVPEQRDALHLPLLSWGVKRPRTRIGGLLDPGTPMDADLVASSTVVWEQDADPFAGTADDFPAFNEEELAWLMELLPQ

SEQ ID NO: 163 - protein, 1062 aa, Xanthomonas campestris pv. armoraciae
  N-terminus (1)..(288)
  Repeat 1 (289)..(322)
  Repeat 2 (323)..(356)
  Repeat 3 (357)..(390)
  Repeat 4 (391)..(424)
  Repeat 5 (425)..(458)
  Repeat 6 (459)..(492)
  Repeat 7 (493)..(526)
  Repeat 8 (527)..(560)
  Repeat 9 (561)..(594)
  Repeat 10 (595)..(628)
  Repeat 11 (629)..(662)
  Repeat 12 (663)..(696)
  Repeat 13 (697)..(730)
  Repeat 14 (731)..(764)
  Repeat 14.5 (765)..(784)
  C-terminus (785)..(1062)
MDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNIGGKQALETVQALLPVLCQAHGLTPQQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASNSGGKQALETVQALLPVLCQAHGLTPQQVVAIASNSGGKQALETVQALLPVLCQAHGLTPEQVVAIASNIGGKQALETVQRLLPVLCQAHGLTPQQVVAIASHDGGKQALETVQRLLPVLCQAHGLTPQQVVAIASNGGGKQALETVQRLLPVLCQAHGLTPEQVVAIASNIGGKQALETVQALLPVLCQAHGLTPQQVVAIASNSGGKQALETVQALLPVLCQAHGLTPEQVVAIASNIGGKQALETVQRLLPVLCQAHGLTPEQVVAIASNGGGKQALETVQRLLPVLCQAHGLTPEQVVAIASNIGGKQALETVQALLPVLCQAHGLTPEQVVAIASNGGGRPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRASSRKRSRSDRAVTGPSAQQSFEVRVPEQRDALHLPLSWRVKRPRTSIGGGLPDPGTPTAADLAASSTVMREQDEDPFAGAADDFPAFNEEELAWLMELLPQ

SEQ ID NO: 164 - DNA, 19 bp, Artificial Sequence
  DNA binding site of AvBs3
tctntaaacctnnccctct

SEQ ID NO: 165 - DNA, 23 bp, Artificial Sequence
  DNA binding site of Hax2
tgttattctcacactctccttat

SEQ ID NO: 166 - DNA, 13 bp, Artificial Sequence
  DNA binding site of Hac3
tacacccnnncat

SEQ ID NO: 167 - DNA, 16 bp, Artificial Sequence
  DNA binding site of Hax4
tacctnnactanatat

(19) United States
(12) Patent Application Publication
(10) Pub. No.: US 2012/0324603 A1
(43) Pub. Date: Dec. 20, 2012

(54) Chimeric Endonucleases and Uses Thereof

(75) Inventors: Andrea Hlubek, Quedlinburg (DE); Christian Biesgen, Quedlinburg (DE); Hans Wolfgang Höffken, Ludwigshafen (DE)

(73) Assignee: BASF Plant Sceience Company GmbH, Ludwigshafen (DE)

(21) Appl. No.: 13/511,727

(22) PCT Filed: Nov. 26, 2010
(86) PCT No.: PCT/IB10/55453

§ 371 Date: May 24, 2012

Related U.S. Application Data
(60) Provisional application No. 61/264,715, filed on Nov. 27, 2009.
(60) Provisional application No. 61/365,809, filed on Jul. 20, 2010.
(60) Provisional application No. 61/365,836, filed on Jul. 20, 2010.

(30) Foreign Application Priority Data
Nov. 27, 2009 (EP) 09177375.4
Jul. 20, 2010 (EP) 10170164.7
Jul. 20, 2010 (EP) 10170199.3

(51) Int. Cl. C12N 9/22; C12N 15/62; C12N 15/63; C07H 21/04; A01H 5/00; C12N 1/19; C12N 5/10; C12N 1/13; C12Q 1/68; C12N 15/87; C12N 15/55; C12N 1/15

(52) U.S. Cl. 800/298; 435/199; 536/23.2; 435/320.1; 536/24.2; 435/418; 435/254.11; 435/254.2; 435/325; 435/366; 435/415; 435/257.2; 435/411; 435/414; 435/417; 435/412; 435/6.1; 435/462

(57) ABSTRACT

The invention relates to chimeric endonucleases, comprising an endonuclease and a heterologous DNA binding domain, as well as methods of targeted integration, targeted deletion or targeted mutation of polynucleotides using chimeric endonucleases.

### FIELD OF THE INVENTION

The invention relates to chimeric endonucleases, comprising a endonuclease and a heterologous DNA binding domain, as well as methods of targeted integration, targeted deletion or targeted mutation of polynucleotides using chimeric endonucleases.

### BACKGROUND OF THE INVENTION

Genome engineering is a common term to summarize different techniques to insert, delete, substitute or otherwise manipulate specific genetic sequences within a genome and has numerous therapeutic and biotechnological applications. More or less all genome engineering techniques use recombinases, integrases or endonucleases to create DNA double strand breaks at predetermined sites in order to promote homologous recombination.

In spite of the fact that numerous methods have been employed to create DNA double strand breaks, the development of effective means to create DNA double strand breaks at highly specific sites in a genome remains a major goal in gene therapy, agrotechnology, and synthetic biology.

One approach to achieve this goal is to use nucleases with specificity for a sequence that is sufficiently large to be present at only a single site within a genome. Nucleases recognizing such large DNA sequences of about 15 to 30 nucleotides are therefore called “meganucleases” or “homing endonucleases” and are frequently associated with parasitic or selfish DNA elements, such as group 1 self-splicing introns and inteins commonly found in the genomes of plants and fungi. Meganucleases are commonly grouped into four families: the LAGLIDADG family, the GIY-YIG family, the His-Cys box family and the HNH family. These families are characterized by structural motifs, which affect catalytic activity and the sequence of their DNA recognition sequences.

Natural meganucleases from the LAGLIDADG family have been used to effectively promote site-specific genome modifications in insect and mammalian cell cultures, as well as in many organisms, such as plants, yeast or mice, but this approach has been limited to the modification of either homologous genes that conserve the DNA recognition sequence or to preengineered genomes into which a recognition sequence has been introduced. In order to avoid these limitations and to promote the systematic implementation of DNA double strand break stimulated gene modification new types of nucleases have been created.

One type of new nucleases consists of artificial combinations of unspecific nucleases to a highly specific DNA binding domain. The effectiveness of this strategy has been demonstrated in a variety of organisms using chimeric fusions between an engineered zinc finger DNA-binding domain and the non-specific nuclease domain of the FokI restriction enzyme (e.g. WO03/089452) a variation of this approach is to use an inactive variant of a meganuclease as DNA binding domain fused to an unspecific nuclease like FokI as disclosed in Lippow et al., “Creation of a type IIS restriction endonuclease with a long recognition sequence”, Nucleic Acid Research (2009), Vol. 37, No. 9, pages 3061 to 3073.

An alternative approach is to genetically engineer natural meganucleases in order to customize their DNA binding regions to bind existing sites in a genome, thereby creating engineered meganucleases having new specificities (e.g WO07093918, WO2008/093249, WO09114321). However, many meganucleases which have been engineered with respect to DNA cleavage specificity have decreased cleavage activity relative to the naturally occurring meganucleases from which they are derived (US2010/0071083). Most meganucleases do also act on sequences similar to their optimal binding site, which may lead to unintended or even detrimental off-target effects. Several approaches have already been taken to enhance the efficiency of meganuclease induced homologous recombination e.g. by fusing nucleases to the ligand binding domain of the rat Glucocorticoid Receptor in order to promote or even induce the transport of this modified nuclease to the cell nucleus and therefore its target sites by the addition of dexamethasone or similar compounds (WO2007/135022). Despite that fact, there is still a need in the art to develop meganucleases having high induction rates of homologous recombination and/or a high specificity for their binding site, thereby limiting the risk of off-target effects.

### BRIEF SUMMARY OF THE INVENTION

The invention provides chimeric endonucleases comprising at least one endonuclease having DNA double strand break inducing activity and at least one heterologous DNA binding domain. Preferably at least one endonuclease of the chimeric endonuclease is a LAGLIDADG endonuclease. In one embodiment, at least one LAGLIDADG endonuclease is I-SceI, I-CreI, I-CeuI, I-ChuI, I-DmoI, PI-SceI, I-MsoI, or I-AniI, or a LAGLIDADG endonuclease having at least 45% amino acid sequence identity to any one of these. In another embodiment of the invention, at least one LAGLIDADG endonuclease has at least 80% amino acid sequence identity to a polypeptide described by SEQ ID NO: 1, 2, 3 or 159. The LAGLIDADG endonuclease may be wild-type, engineered, optimized or optimized engineered LAGLIDADG endonucleases.

The heterologous DNA binding domain is preferably a transcription factor or an inactive nuclease, or a fragment comprising a DNA binding domain of a transcription factor or a nuclease. In one embodiment at least one heterologous DNA binding domain is an inactive I-SceI, I-CreI, I-CeuI, I-ChuI, I-DmoI, Pi-SceI, I-MsoI, or I-AniI or an inactive homolog of these having at least 45% amino acid sequence identity. In one embodiment the heterologous DNA binding domain is an inactive version of a LAGLIDADG endonucleases having an amino acid sequence as described by at least one of SEQ ID NO: 1, 2, 3, 5, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 142 or 159, preferably having an amino acid sequence as described by any one of SEQ ID NO: 1, 2, 3, 5 or 159.

In another embodiment of the invention the heterologous DNA binding domain is a transcription factor or an DNA binding domain of a transcription factor. Preferably the transcription factor or the DNA binding domain of a transcription factor comprises a HTH domain. Even more preferred, the transcription factor or the DNA binding domain of a transcription factor comprises a HTH domain comprising an amino acid sequence of at least 80% sequence identity to at least one amino acid sequence described by SEQ ID NO: 91, 92, 93, 94, 95, 96, 97, 98, 99, 100, 101, 102, 103, 104, 105, 106, 107, 108, 109, 110, 111, 112, 113, 114, 115, 116, 117, 118 or 119, preferably described by 91, 92, 93, 94, 95, 112, 113, 114, 115, 116, 117, 118 or 119. In one embodiment of the invention, the heterologous DNA binding domain comprises a polypeptide having at least 80% amino acid sequence identity to a polypeptide described by SEQ ID NO: 6, 7 or 8. Preferably the chimeric endonuclease comprises a linker (or synonymous linker polypeptide) to connect at least one endonuclease with at least one heterologous DNA binding domain. The chimeric endonuclease may comprise one or more NLS-sequences or one or more SecIII or SecIV secretion signals or a combination of one or more NLS-sequences and one or more SecIII or SecIV secretion signals or a combination of one or more SecIII and SecIV secretion signals with one or more NLS-sequences. In one embodiment of the invention the DNA binding activity of the heterologous DNA binding domain is inducible. In another embodiment of the invention, the DNA double strand break inducing activity of the endonulcease is inducible by expression of the second monomer of a homo- or heterodimeric endonuclease, preferably a homo- or heterodimeric LAGLIDADG endonuclease. The chimeric endonucleases may comprise at least one NLS-sequence or at least one SecIII or at least one SecIVsecretion signal or a combination of one or more NLS-sequences, one or more SecIII secretion signals or one or more SecIV secretion signals.

The invention does further provide isolated polynucleotides coding for a chimeric endonuclease. Preferably the isolated polynucleotide coding for a chimeric endonuclease is codon optimized, or has a low content of RNA instability motifes, or has a low content of cryptic splice sites, or has a low content of alternative start codons, or has a low content of restriction sites, or has a low content of RNA secondary structures, or has a combination of the features described above. A further embodiment of the invention is an expression cassette comprising an isolated polynucleotide coding for a chimeric endonuclease in functional combination with a promoter and an terminator sequence. An additional group of isolated polynucleotides provided by the invention are isolated polynucleotides comprising a chimeric recognition sequence having a length of about 15 to about 300 nucleotides and comprising a recognition sequence of an endonuclease and a recognition sequence of a heterologous DNA binding domain. Preferably the chimeric recognition sequence comprises a DNA recognition sequence of a LAGLIDADG endonuclease, even more preferred a DNA recognition sequence of a LAGLIDADG endonuclease having an amino acid sequence as described by at least one of SEQ ID NOs: 1, 2, 3, 5, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 142 or 159, preferably having an amino acid sequence as described by SEQ ID NO: 1, 2, 3, 5 or 159. In a further embodiment of the invention, the chimeric recognition site comprises a DNA recognition sequence of I-SceI, I-CreI, I-DmoI, I-MsoI, I-CeuI, I-ChuI, Pi-SceI or I-AniI, and a recognition sequence of a heterologous DNA binding domain having at least 50% sequence amino acid sequence identity to scTet, scArc, LacR, MerR or MarA or to a DNA binding domain fragment of scTet, scArc, LacR, MerR or MarA. Preferred polynucleotides provided by the invention comprise a chimeric recognition sequence, comprising a DNA recognition sequence of I-SceI and a recognition sequence of scTet or scArc, wherein the DNA recognition sequence of I-SceI and the recognition sequence of scTet or scArc are directly connected, or are connected via a linker sequence of 1 to 10 nucleotides. In a preferred embodiment the isolated polynucleotide comprises a chimeric recognition sequence comprising a polynucleotide sequence as described by any one of SEQ ID NOs: 14, 15, 16, 17, 18, 19 or 20.

The invention does further provide a vector, host cell or non human organism comprising an isolated polynucleotide coding for a chimeric endonuclease, or an isolated polynucleotide as described above, or an expression cassette, or an isolated polynucleotide comprising a chimeric recognition sequence or a chimeric endonuclease or comprising a combination of one or more of these. Preferably the non-human organism is a plant.

The invention provides methods of using the chimeric endonucleases and chimeric recognition sequences described herein to induce or facilitate homologous recombination or end joining events. Preferably methods for targeted integration or excision of sequences. Preferably the sequences being excised are marker genes.

One embodiment of the invention is a method for providing a chimeric endonuclease, comprising the steps of: a) providing at least one endonuclease coding region, b) providing at least one heterologous DNA binding domain coding region, c) providing a polynucleotide having a potential DNA recognition sequence or potential DNA recognition sequences of the endonuclease or endonucleases of step a) and having a potential recognition sequence or having potential recognition sequences of the heterologous DNA binding domain or heterologous DNA binding domains of step b), d) creating a translational fusion of the coding regions of all endonucleases of step b) and all heterologous DNA binding domains of step c), e) expressing a chimeric endonuclease from the translational fusion created in step d), f) testing the chimeric endonuclease expressed in step e) for cleavage of the polynucleotide of step c).

The invention does further provide a method for homologous recombination of polynucleotides comprising the following steps: a) providing a cell competent for homologous recombination, b) providing a polynucleotide comprising a chimeric recognition site flanked by a sequence A and a sequence B, c) providing a polynucleotide comprising sequences A′ and B′, which are sufficiently long and homologous to sequence A and sequence B, to allow for homologous recombination in said cell and d) providing a chimeric endonuclease as described herein or an expression cassette as described herein, e) combining b), c) and d) in said cell and f) detecting recombined polynucleotides of b) and c), or selecting for or growing cells comprising recombined polynucleotides of b) and c). Preferably the method for homologous recombination of polynucleotides leads to a homologous recombination, wherein a polynucleotide sequence comprised in the competent cell of step a) is deleted from the genome of the growing cells of step f). A further method of the invention is a method for targeted mutation comprising the following steps: a) providing a cell comprising a polynucleotide comprising a chimeric recognition site of an chimeric endonuclease, b) providing an chimeric endonuclease being able to cleave the chimeric recognition site of step a), c) combining a) and b) in said cell and d) detecting mutated polynucleotides, or selecting for growing cells comprising mutated polynucleotides. In another preferred embodiment of the invention, the methods described above comprise a step, wherein the chimeric endonuclease and the chimeric recognition site are combined in at least one cell via crossing of organisms, via transformation or via transport mediated via a Sec III or SecIV peptide fused to the optimized endonuclease.

### BRIEF DESCRIPTION OF THE FIGURES

FIG. 1 depicts a sequence alignment of different I-SceI homologs, wherein 1 is SEQ ID NO: 1, 2 is SEQ ID NO: 56, 3 is SEQ ID NO: 57, 4 is SEQ ID NO: 58, 5 is SEQ ID NO: 59.

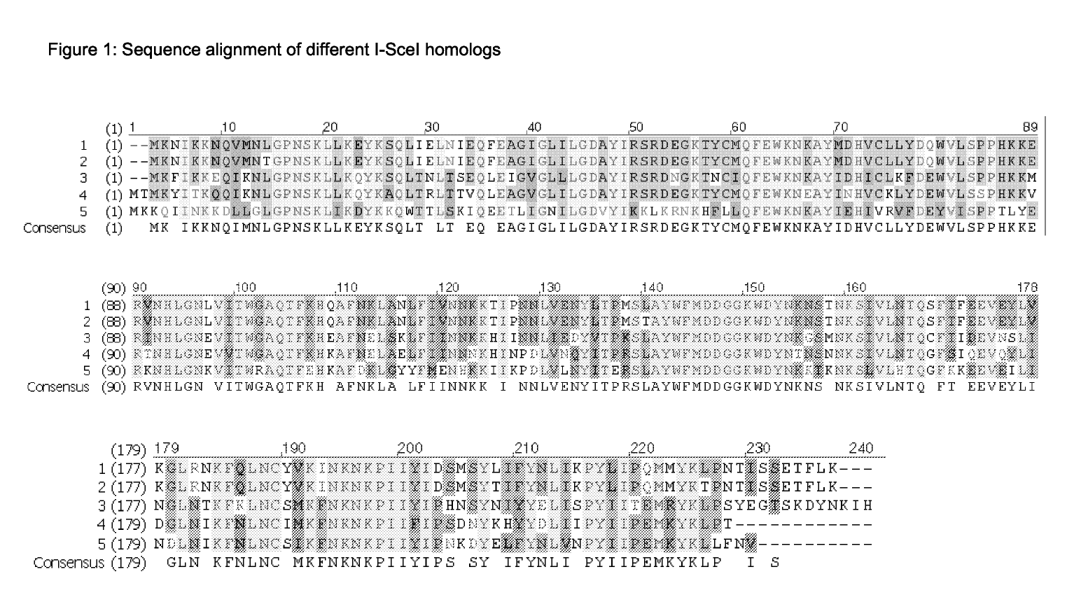

FIG. 2 depicts a sequence alignment of different I-CreI homologs, wherein 1 is SEQ ID NO: 60, 2 is SEQ ID NO: 61, 3 is SEQ ID NO: 62, 4 is SEQ ID NO: 63, 5 is SEQ ID NO: 64.

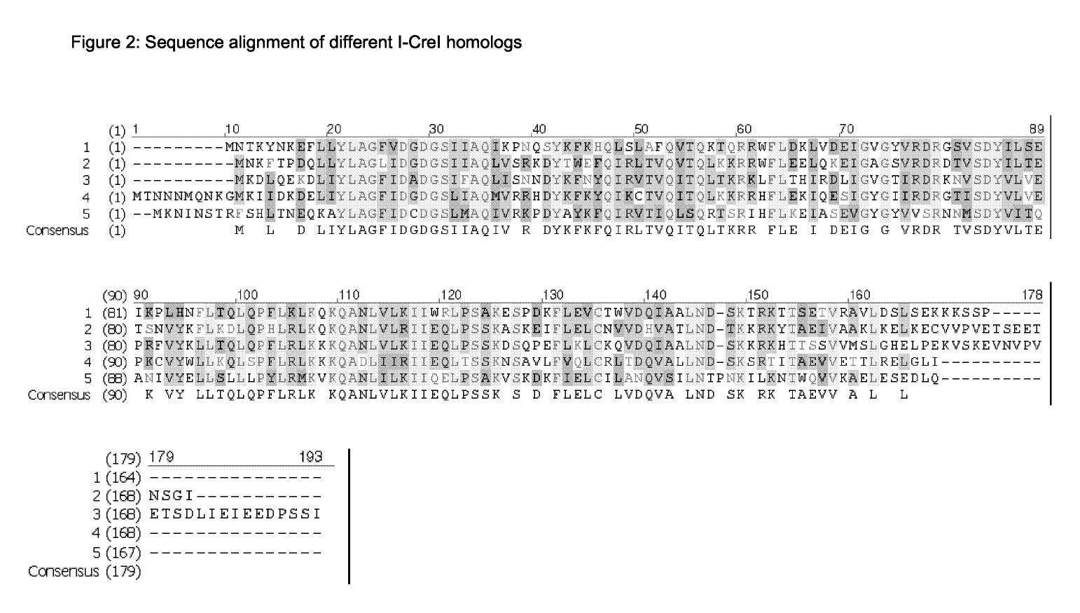

FIGS. 3a to 3c depicts a sequence alignment of different PI-SceI homologs, wherein 1 is SEQ ID NO: 79, 2 is SEQ ID NO: 80, 3 is SEQ ID NO: 81, 4 is SEQ ID NO: 82, 5 is SEQ ID NO: 83.

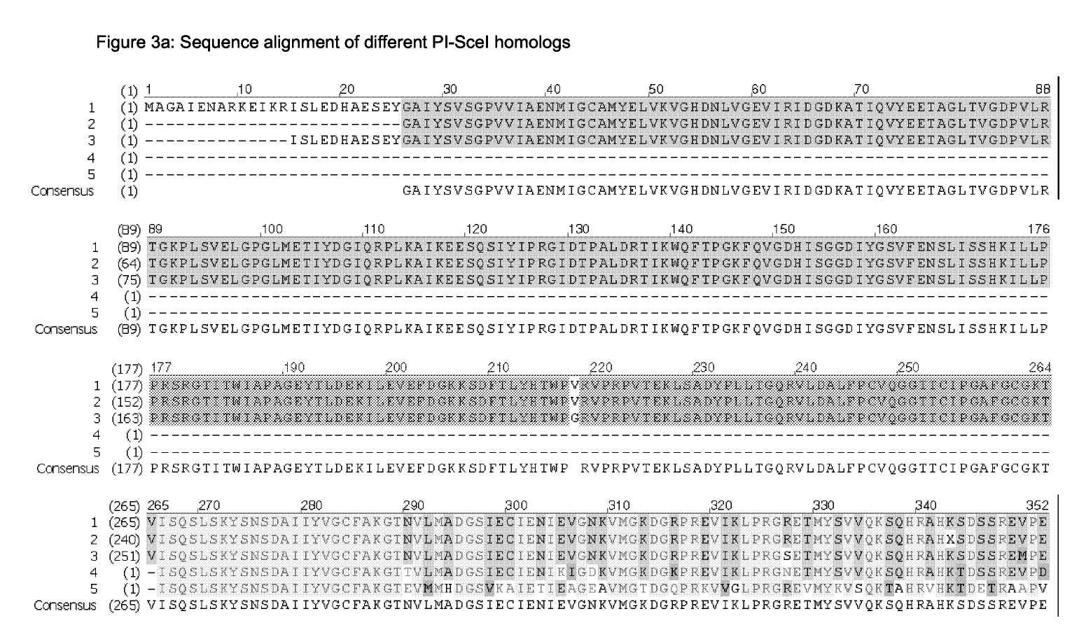

FIG. 4 depicts a sequence alignment of different I-CeuI homologs, wherein 1 is SEQ ID NO: 65, 2 is SEQ ID NO: 66, 3 is SEQ ID NO: 67, 4 is SEQ ID NO: 68, 5 is SEQ ID NO: 69.

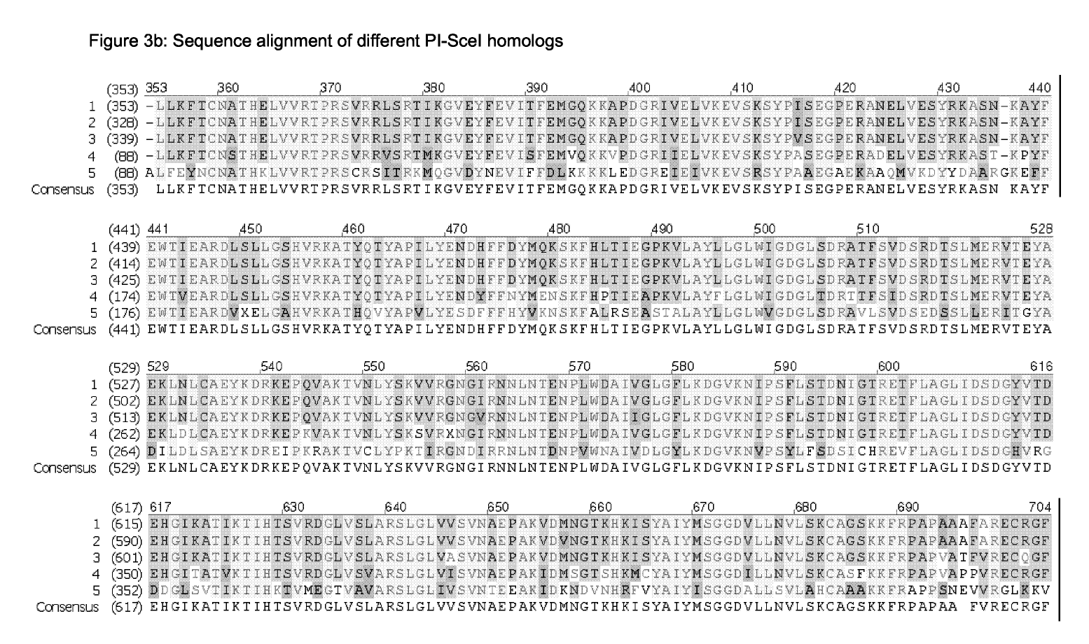

FIG. 5 depicts a sequence alignment of different I-ChuI homologs, wherein 1 is SEQ ID NO: 70, 2 is SEQ ID NO: 71, 3 is SEQ ID NO: 72, 4 is SEQ ID NO: 73, 5 is SEQ ID NO: 74.

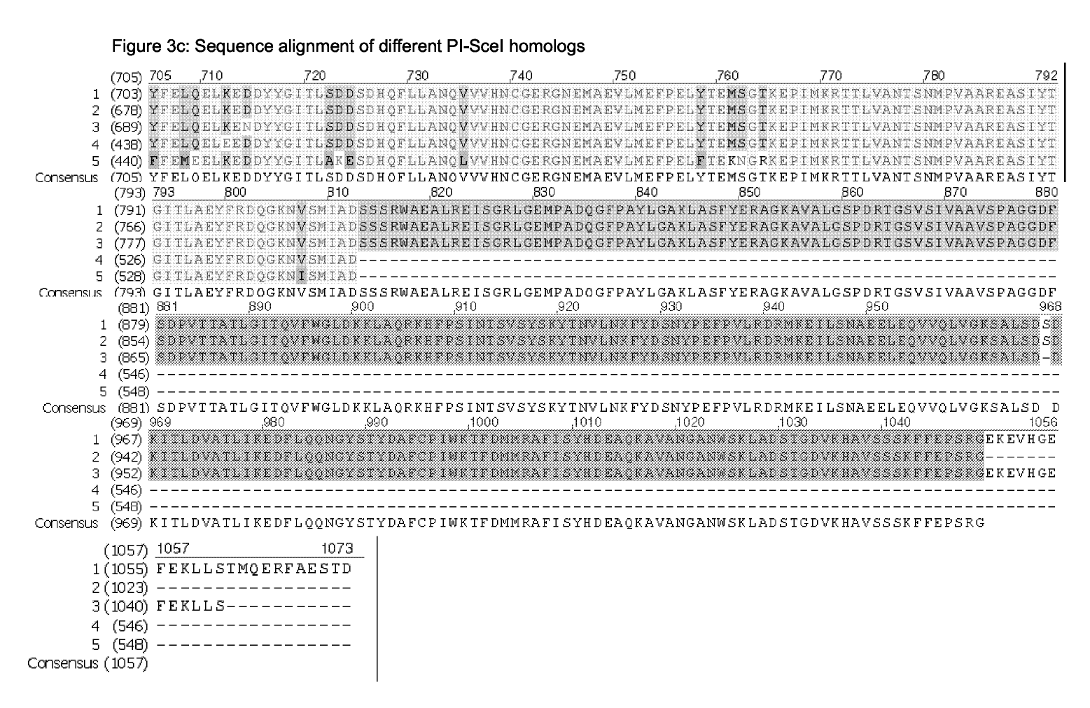

FIG. 6 depicts a sequence alignment of different I-DmoI homologs, wherein 1 is SEQ ID NO: 75, 2 is SEQ ID NO: 76, 3 is SEQ ID NO: 77, 4 is SEQ ID NO: 78.

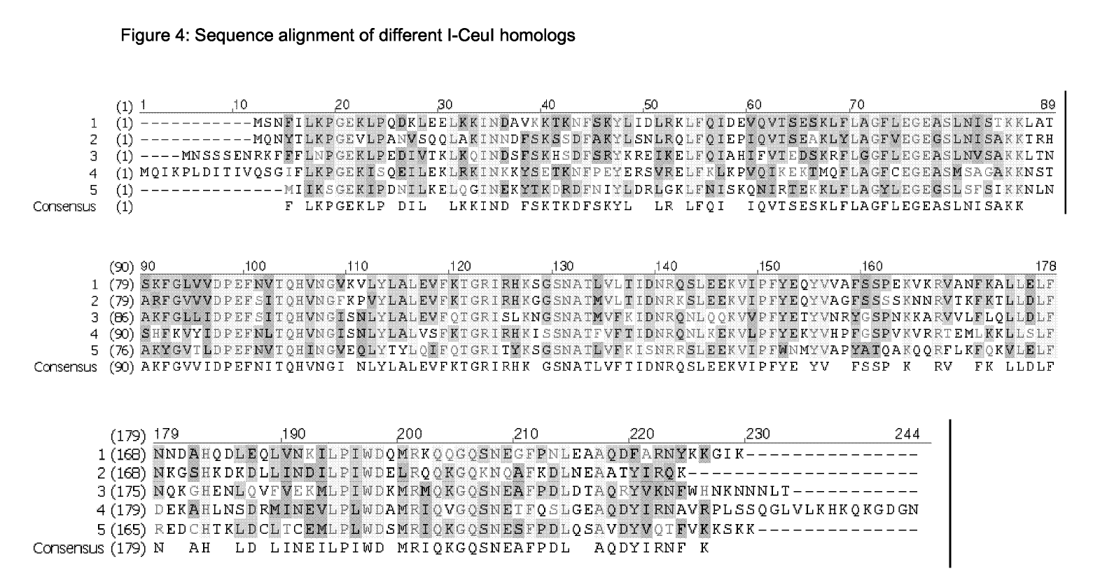

FIG. 7 depicts a sequence alignment of different I-MsoI homologs, wherein 1 is SEQ ID NO: 84 and 2 is SEQ ID NO: 85.

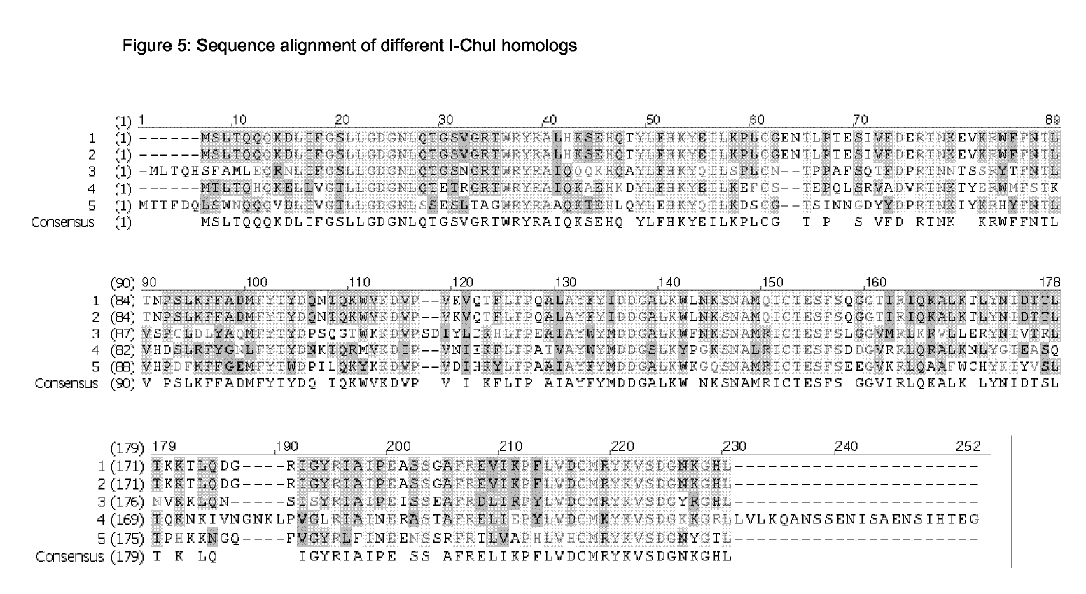

FIG. 8 depicts a sequence alignment of different TetR homologs, wherein 1 is SEQ ID NO: 86, 2 is SEQ ID NO: 87, 3 is SEQ ID NO: 88, 4 is SEQ ID NO: 89, 5 is SEQ ID NO: 90.

FIG. 9a depicts a sequence alignment of HTH domains of different TetR homologs, wherein 1 is SEQ ID NO: 91, 2 is SEQ ID NO: 92, 3 is SEQ ID NO: 93, 4 is SEQ ID NO: 94, 5 is SEQ ID NO: 95.

FIG. 9b depicts a sequence alignment of HTH domains of different ArcR homologs, wherein 1 is SEQ ID NO: 96, 2 is SEQ ID NO: 97, 3 is SEQ ID NO: 98, 4 is SEQ ID NO: 99, 5 is SEQ ID NO: 100.

FIG. 10a depicts a sequence alignment of HTH domains of different LacR homologs, wherein 1 is SEQ ID NO: 101, 2 is SEQ ID NO: 102, 3 is SEQ ID NO: 103, 4 is SEQ ID NO: 104, 5 is SEQ ID NO: 105.

FIG. 10b depicts a sequence alignment of HTH domains of different MerR homologs, wherein 1 is SEQ ID NO: 106, 2 is SEQ ID NO: 107, 3 is SEQ ID NO: 108, 4 is SEQ ID NO: 109, 5 is SEQ ID NO: 110, 6 is SEQ ID NO: 111.

FIG. 11 depicts a sequence alignment of HTH domains of different MarA homologs, wherein 1 is SEQ ID NO: 112, 2 is SEQ ID NO: 113, 3 is SEQ ID NO: 114, 4 is SEQ ID NO: 115, 5 is SEQ ID NO: 1116, 6 is SEQ ID NO: 117, 7 is SEQ ID NO: 118, 8 is SEQ ID NO: 119.

FIG. 12 depicts a sequence alignment of different MarA homologs, wherein 1 is SEQ ID NO: 120, 2 is SEQ ID NO: 121, 3 is SEQ ID NO: 122, 4 is SEQ ID NO: 123, 5 is SEQ ID NO: 124, 6 is SEQ ID NO: 125, 7 is SEQ ID NO: 126, 8 is SEQ ID NO: 127.

### DESCRIPTION OF THE INVENTION

The invention provides chimeric endonucleases, which can be used as alternative DNA double strand break inducing enzymes. The invention also includes methods of using these chimeric endonucleases.

### Chimeric Endonucleases of the Invention

The chimeric endonucleases of the invention comprise at least one endonuclease having DNA double strand break inducing activity and at least one heterologous DNA binding domain.

### The Endonuclease

Endonucleases suitable for the invention induce DNA double strand breaks in a DNA recognition sequence of at least 4, at least 6, at least 8, at least 10, at least 14, at least 16, at least 18 or at least 20 base pairs.

Preferred endonucleases induce double strand breaks in a DNA recognition sequence of at least 14 base pairs, more preferred of at least 16 base pairs, even more preferred of at least 18 base pairs.

The term “DNA recognition sequence” generally refers to those sequences which, under the conditions in a cell e.g. in a plant cell, enables recognition and cleavage by the endonuclease. Examples for DNA recognition sequences as well as endonucleases cutting those DNA recognition sequences can be found in Table 8 below.

Many different endonucleases are known to the person skilled in the art. Examples are homing endonucleases such as: F-SceI, F-SceII, F-SuvI, F-TevII, I-AmaI, I-AniI, I-CeuI, I-CeuAIIP, I-ChuI, I-CmoeI, I-CpaI, I-CpaII, I-CreI, I-CrepsbIP, I-CrepsbIIP, I-CrepsbIIIP, I-CrepsbIVP, I-CsmI, I-CvuI, I-CvuAIP, I-DdiI, I-DdiII, I-DirI, I-DmoI, I-HmuI, I-HspNIP, I-LlaI, I-MsoI, I-NaaI, I-Nan I, I-NcIIP, I-NgrIP, I-NitI, I-NjaI, I-Nsp236IP, I-PakI, I-PboIP, I-PcuIP, I-PcuAI, I-PcuVI, I-PgrIP, I-PobIP, I-Port, I-PorIIP, I-PpbIP, I-PpoI, I-SPBetaIP, I-ScaI, I-SceI, I-SceII, I-SceIII, I-SceIV, I-SceV, I-SceVI, I-SceVII, I-SexOP, I-SneIP, I-SpomCP, I-SpomIP, I-SpomIIP, I-SquIP, I-Ssp6803I, I-SthPhiJP, I-SthPhiST3P, I-SthPhiS3bP, I-TdeIP, I-TevI, I-TevII, I-TevIII, I-UarAP, I-UarHGPA1P, I-UarHGPA13P, I-VinIP, I-ZbiIP, PI-MtuI, PI-MtuHIP, PI-MtuHIIP, PI-PfuI, PI-PfuII, PI-PkoI, PI-PkoII, PI-PspI, PI-Rma43812IP, PI-SPBetaIP, PI-SceI, PI-TfuI, PI-TfuII, PI-ThyI, PI-TliI, PI-TliII, H-DreI, I-BasI, I-BmoI, I-PogI, I-TwoI, PI-MgaI, PI-PabI, PI-PabII.

Preferred homing endonucleases are GIY-YIG-, His-Cys box-, HNH- or LAGLIDADG-endonucleases. The GIY-YIG endonucleases have a GIY-YIG module of 70 to 100 amino acids length, which includes four or five conserved sequence motifs with four invariant residues (Van Roey et al (2002), Nature Struct. Biol. 9:806 to 811). His-Cys box endonucleases comprise a highly conserved sequence of histidines and cysteines over a region of several hundred amino acid residues. The HNH-endonucleases are defined by sequence motifs containing two pairs of conserved histidines surrounded by asparagine residues. Further information on His-Cys box- and HNH endonucleases is provided by Chevalier et al. (2001), Nucleic Acids Res. 29(18): 3757 to 3774).

Preferably, the homing endonuclease used in the chimeric endonucleases belongs to the group of LAGLIDADG endonucleases.

LAGLIDADG endonucleases can be found in the genomes of algae, fungi, yeasts, protozoan, chloroplasts, mitochondria, bacteria and archaea. LAGLIDADG endonucleases comprise at least one conserved LAGLIDADG motif. The name of the LAGLIDADG motif is based on a characteristic amino acid sequence appearing in all LAGLIDADG endonucleases. The term LAGLIDADG is an acronym of this amino acid sequence according to the one-letter-code as described in the STANDARD ST.25 i.e. the standard adopted by the PCIPI Executive Coordination Committee for the presentation of nucleotide and amino acid sequence listings in patent applications.

However, the LAGLIDADG motif is not fully conserved in all LAGLIDADG endonucleases, (see for example Chevalier et al. (2001), Nucleic Acids Res. 29(18): 3757 to 3774, or Dalgaard et al. (1997), Nucleic Acids Res. 25(22): 4626 to 4638), so that some LAGLIDADG endonucleases comprise some amino acid changes in their LAGLIDADG motif. LAGLIDADG endonucleases comprising only one LAGLIDADG motif usually act as homo- or heterodimers. LAGLIDADG endonucleases comprising two LAGLIDADG motifs act as monomers and comprise usually a pseudo-dimeric structure.

LAGLIDADG endonucleases can be isolated for example from polynucleotides of organisms mentioned for exemplary purposes in Table 1, 2, 3, 4, 5 and 6, or de novo synthesized by techniques known in the art, e.g. using sequence information available in public databases known to the person skilled in the art, for example Genbank Benson (2010), Nucleic Acids Res 38:D46-51 or Swissprot Boeckmann (2003), Nucleic Acids Res 31:365-70

A collection of LAGLIDADG endonucleases can be found in the PFAM-Database for protein families. The PFAM-Database accession number PF00961 describes the LAGLIDADG 1 protein family, which comprises about 800 protein sequences. PFAM-Database accession number PF03161 describes members of the LAGLIDADG 2 protein family, comprising about 150 protein sequences. An alternative collection of LAGLIDADG endonucleases can be found in the InterPro data base, e.g. InterPro accession number IPR004860.

The term LAGLIDADG endonucleases shall also encompass artificial homo- and heterodimeric LAGLIDADG endonucleases, which can be created e.g. by modifying the protein-protein interaction regions of the monomers in order to promote homo- or heterodimer formation. Examples of artificial heterodimeric LAGLIDADG endonuclease comprising the LAGLIDADG endonuclease I-Dmo I as one domain can be found in WO2009/074842 and WO2009/074873.

In addition to that, the term LAGLIDADG endonucleases shall also encompass artificial single chain endonucleases, which can be created by making translational fusions of monomers of homo- or heterodimeric LAGLIDADG endonucleases.

Accordingly in one embodiment of the invention, the chimeric endonucleases of the invention comprise at least one LAGLIDADG endonuclease.

In further embodiments the LAGLIDADG endonuclease comprised in the chimeric endonuclease can be a monomeric, homodimeric, artificial homo- or heterodimeric or artificial single chain LAGLIDADG endonuclease.

In one embodiment the LAGLIDAG endonuclease is a monomeric, homodimeric, heterodimeric, or artificial single chain LAGLIDADG endonuclease. Preferably the endonuclease is a monomeric or artificial single chain LAGLIDADG endonuclease.

Preferred LAGLIDADG endonucleases are: I-AniI, I-Sce I, I-Chu I, I-Dmo I, I-Cre I, I-Csm I, PI-Sce I, PI-Tli I, PI-Mtu I, I-Ceu I, I-Sce II, I-Sce III, HO, PI-Civ I, PI Ctr I, PI-Aae I, PI-Bsu I, PI-Dha I, PI-Dra I, PI-Mav I, PI-Mch I, PI-Mfu I, PI-Mfl I, PI-Mga I, PI-Mgo I, PI-Min I, PI-Mka I, PI-Mle I, PI-Mma I, PI-Msh I, PI-Msm I, I-Mso I, PI-Mth I, PI-Mtu I, PI-Mxe I, PI-Npu I, PI-Pfu I, PI-Rma I, PI-Spb I, PI-Ssp I, PI-Fac I, PI-Mja I, PI-Pho I, PI-Tag I, PI-Thy I, PI-Tko I, and PI-Tsp I and homologs of any one of these having at least 49%, 51%, 58%, 60%, 70%, 80%, 85%, 90%, 92%, 93%, 94%, 95%, 96%, 97%, 98% or 99% sequence identity on amino acid level; more preferred are: I-Sce I, I-Chu I, I-Dmo I, I-Cre I, I-Csm I, PI-Pfu I, PI-Sce I, PI-Tli I, I-Mso I, PI-Mtu I, I-Ceu I, I-Sce II, I-Sce III, and HO and homologs of any one of these having at least 49%, 51%, 58%, 60%, 70%, 80%, 85%, 90%, 92%, 93%, 94%, 95%, 96%, 97%, 98% or 99% sequence identity on amino acid level; even more preferred are, I-Sce I, I-Chu I, I-Dmo I, I-Cre I, I-Csm I, PI-Sce I, PI-Pfu I, PI-Tli I, I-Mso I, PI-Mtu I and I-Ceu I and homologs of any one of these having at least 49%, 51%, 58%, 60%, 70%, 80%, 85%, 90%, 92%, 93%, 94%, 95%, 96%, 97%, 98% or 99% sequence identity on amino acid level; still more preferred are I-Dmo I, I-Cre I, I-Sce I, I-Mso I and I-Chu I and homologs of any one of these having at least 49%, 51%, 58%, 60%, 70%, 80%, 85%, 90%, 92%, 93%, 94%, 95%, 96%, 97%, 98% or 99% sequence identity on amino acid level, most preferred is I-Sce I and homologs of I-Sce I having at least 49%, 51%, 58%, 60%, 70%, 80%, 85%, 90%, 92%, 93%, 94%, 95%, 96%, 97%, 98% or 99% sequence identity on amino acid level.

Preferred monomeric LAGLIDADG endonucleases are: I-AniI, I-Sce I, I-Chu I, I-Dmo I, I-Csm I, PI-Sce I, PI-Tli I, PI-Mtu I, I-Sce II, I-Sce III, HO, PI-Civ I, PI Ctr I, PI-Aae I, PI-Bsu I, PI-Dha I, PI-Dra I, PI-Mav I, PI-Mch I, PI-Mfu I, PI-Mfl I, PI-Mga I, PI-Mgo I, PI-Min I, PI-Mka I, PI-Mle I, PI-Mma I, PI-Msh I, PI-Msm I, PI-Mth I, PI-Mtu I, PI-Mxe I, PI-Npu I, PI-Pfu I, PI-Rma I, PI-Spb I, PI-Ssp I, PI-Fac I, PI-Mja I, PI-Pho I, PI-Tag I, PI-Thy I, PI-Tko I, and PI-Tsp I; and homologs of any one these having at least 49%, 51%, 58%, 60%, 70%, 80%, 85%, 90%, 92%, 93%, 94%, 95%, 96%, 97%, 98% or 99% sequence identity on amino acid level.

More preferred monomeric LAGLIDADG endonucleases are: I-Sce I, I-Chu I, I-Dmo I, I-Csm I, PI-Pfu I, PI-Sce I, PI-Tli I, PI-Mtu I, I-Sce II, I-Sce III, and HO and homologs of any one of these having at least 49%, 51%, 58%, 60%, 70%, 80%, 85%, 90%, 92%, 93%, 94%, 95%, 96%, 97%, 98% or 99% sequence identity on amino acid level.

Even more preferred monomeric LAGLIDADG endonucleases are: I-Sce I, I-Chu I, I-Dmo I, I-Csm I, PI-Sce I, PI-Tli I, and PI-Mtu I; homologs of any one these having at least 49%, 51%, 58%, 60%, 70%, 80%, 85%, 90%, 92%, 93%, 94%, 95%, 96%, 97%, 98% or 99% sequence identity on amino acid level.

Still more preferred monomeric LAGLIDADG endonucleases are: I-Dmo I, I-Sce I, and I-Chu I; homologs of any one these having at least 49%, 51%, 58%, 60%, 70%, 80%, 85%, 90%, 92%, 93%, 94%, 95%, 96%, 97%, 98% or 99% sequence identity on amino acid level.

One type of homolog LAGLIDADG endonucleases are artificial single chain LAGLIDADG endonucleases, which may comprise two sub-units of the same LAGLIDADG endonuclease, such as single-chain I-Cre, single-chain I-Ceu I or single-chain I-Ceu II as disclosed in WO03078619, or which may comprise two sub-units of different LAGLIDADG endonucleases. Artificial single chain LAGLIDADG endonucleases, which comprise two sub-units of different LAGLIDADG endonucleases are called hybrid meganucleases.

Preferred artificial single chain LAGLIDADG endonucleases are single-chain I-CreI, single-chain I-CeuI or single-chain I-CeuII and hybrid meganucleases like: I-Sce/I-Chu I, I-Sce/PI-Pfu I, I-Chu/I-Sce I, I-Chu/PI-Pfu I, I-Sce/I-Dmo I, I Dmo I/I-See I, I-Dmo I/PI-Pfu I, I-DmoI/I-Cre I, I-Cre I/I-Dmo I, I-Cre I/PI-Pfu I, I-Sce I/I-Csm I, I-Sce I/I-Cre I, I-Sce I/PI-Sce I, I-Sce I/PI-TliI, I-Sce I/PI-Mtu I, I-Sce I/I-Ceu I, I-Cre I/I-Ceu I, I-Chu I/I-Cre I, I-Chu I/I-Dmo I, I-Chu I/I-Csm I, I-Chu I/PI-Sce I, I-Chu I/PI-Tli I, I-Chu I/PI-Mtu I, I-Cre I/I-Chu I, I-Cre I/I-Csm I, I-Cre I/PI-Sce I, I Cre I/PI-Tli I, I-Cre I/PI-Mtu I, I-Cre I/I-Sce I, I-Dmo I/I-Chu I, I-Dmo I/I-Csm I, I Dmo I/PI-Sce I, I-Dmo I/PI-Tli I, I-Dmo I/PI-Mtu I, I-Csm I/I-Chu I, I-Csm I/PI-Pfu I, I-Csm I/I-CreI, I-Csm I/I-DmoI, I-Csm I/PI-SceI, I-Csm I/PI-Tli I, I-Csm I/PI-Mtu I, I-Csm I/I-Sce I, PI-Sce I/I-Chu I, PI-Sce I/I-Pfu I, PI-Sce I/I-Cre I, PI-Sce I/I Dmo I, PI-Sce I/I-Csm I, PI-Sce I/PI-Tli I, PI-Sce I/PI-Mtu I, PI-Sce I/I-Sce I, PI-Tli I/I Chu I, PI-Tli I/PI-Pfu I, PI-Tli I/I-Cre I, PI-Tli I/I-Dmo I, PI-Tli I/I-Csm I, PI-Tli I/PI Sce I, PI-Tli I/PI-Mtu I, PI-Tli 1/I-Sce I, PI-Mtu I/I-Chu I, PI-Mtu I/PI-Pfu I, PI-Mtu I/I-Cre I, PI-Mtu I/I-Dmo I, PI-Mtu I/I-Csm I, PI-Mtu I/I-Sce I, PI-Mtu I/PI-Tli I, and PI-Mtu I/I-SceI disclosed in WO03078619, in WO09/074,842, WO2009/059195 and in WO09/074,873, as well as LIG3-4SC being disclosed in WO09/006,297, or single chain I-Cre I V2 V3 being disclosed in Sylvestre Grizot et al., “Efficient targeting of a SCID gene by an engineered single-chain homing endonuclease”, Nucleic Acids Research, 2009, Vol. 37, No. 16, pages 5405 to 5419. A particular preferred single chain LAGLIDADG endonuclease is single-chain I-Cre I.

Preferred dimeric LAGLIDADG endonucleases are: I-Cre I, I-Ceu I, I-Sce II, I-Mso I and I-Csm I and homologs of any one these having at least 49%, 51%, 58%, 60%, 70%, 80%, 85%, 90%, 92%, 93%, 94%, 95%, 96%, 97%, 98% or 99% sequence identity on amino acid level.

Preferred heterodimeric LAGLIDADG endonucleases are disclosed in WO 07/034,262, WO 07/047,859 and WO08093249.

Homologs of LAGLIDADG endonucleases can for example be cloned from other organisms or can be created by mutating LAGLIDADG endonucleases, e.g. by replacing, adding or deleting amino acids of the amino acid sequence of a given LAGLIDADG endonuclease, which preferably has no effect on its DNA-binding-affinity, its dimer formation affinity or will change its DNA recognition sequence.

As used herein, the term “DNA-binding affinity” means the tendency of a meganuclease or LAGLIDADG endonuclease to non-covalently associate with a reference DNA molecule (e.g. a DNA recognition sequence or an arbitrary sequence). Binding affinity is measured by a dissociation constant, K_{D} (e.g., the K_{D} of I-CreI for the WT DNA recognition sequence is approximately 0.1 nM). As used herein, a meganuclease has “altered” binding affinity if the K_{D} of the recombinant meganuclease for a reference DNA recognition sequence is increased or decreased by a statistically significant (p<0.05) amount relative to a reference meganuclease or LAGLIDADG endonuclease.

As used herein with respect to meganuclease monomers or LAGLIDADG endonuclease monomers, the term “affinity for dimer formation” means the tendency of a monomer to non-covalently associate with a reference meganuclease monomer or LAGLIDADG endonuclease monomer. The affinity for dimer formation can be measured with the same monomer (i.e., homodimer formation) or with a different monomer (i.e., heterodimer formation) such as a reference wild-type meganuclease or a reference LAGLIDADG endonuclease. Binding affinity is measured by a dissociation constant, K_{D}. As used herein, a meganuclease has “altered” affinity for dimer formation, if the K_{D} of the recombinant meganuclease monomer or the recombinant LAGLIDADG endonuclease monomer for a reference meganuclease monomer or for a reference LAGLIDADG endonuclease is increased or decreased by a statistically significant (p<0.05) amount relative to a reference meganuclease monomer or the reference LAGLIDADG endonuclease monomer.

As used herein, the term “enzymatic activity” refers to the rate at which a meganuclease e.g. a LAGLIDADG endonuclease cleaves a particular DNA recognition sequence. Such activity is a measurable enzymatic reaction, involving the hydrolysis of phospho-diester-bonds of double-stranded DNA. The activity of a meganuclease acting on a particular DNA substrate is affected by the affinity or avidity of the meganuclease for that particular DNA substrate which is, in turn, affected by both sequence-specific and non-sequence-specific interactions with the DNA.

For example, it is possible to add nuclear localization signals to the amino acid sequence of a LAGLIDADG endonuclease and/or change one or more amino acids and/or delete parts of its sequence, e.g. parts of the N-terminus or parts of its C-terminus.

For example, it is possible to create a homolog LAGLIDADG endonuclease of I-SceI, by mutating amino acids of its amino acid sequence. Mutations which have little effect on the DNA binding affinity of I-SceI, or will change its DNA recognition sequence are: A36G, L40M, L40V, I41S, I41N, L43A, H91A and I123L.

In one embodiment of the invention, the homologs of LAGLIDADG endonucleases are being selected from the groups of artificial single chain LAGLIDADG endonucleases, including or not including hybrid meganucleases, homologs which can be cloned from other organisms, engineered endonucleases or optimized nucleases.

In one embodiment, the LAGLIDADG endonuclease is selected from the group comprising: I-Sce I, I-Cre I, I-Mso I, I-Ceu I, I-Dmo I, I-Ani I, PI-Sce I, I-Pfu I or homologs of any one these having at least 49%, 51%, 58%, 60%, 70%, 80%, 85%, 90%, 92%, 93%, 94%, 95%, 96%, 97%, 98% or 99% sequence identity on amino acid level.

In another embodiment the LAGLIDADG endonuclease is selected from the group comprising: I-Sce I, I-Chu I, I-Cre I, I-Dmo I, I-Csm I, PI-Sce I, PI-Pfu I, PI-Tli I, PI-Mtu I, and I-Ceu I and homologs of any one these having at least 49%, 51%, 58%, 60%, 70%, 80%, 85%, 90%, 92%, 93%, 94%, 95%, 96%, 97%, 98% or 99% sequence identity on amino acid level.

TABLE 1
Exemplary homologs of I-SceI, which can be
cloned from other organisms.

| Uni-Prot | | SEQ ID | Amino Acid |
|---|---|---|---|
| Accession Nr. | Organism | NO: | Sequence Identity to I-SceI |
| A7LCP1 | S. cerevisiae | 1 | 100 |
| Q36760 | S. cerevisiae | 56 | 98 |
| O63264 | Z. bisporus | 57 | 72 |
| Q34839 | K. thermotolerans | 58 | 71 |
| Q34807 | P. canadensis | 59 | 58 |

TABLE 2
Exemplary homologs of I-CreI, which can be
cloned from other organisms.

| Uni-Prot | | | Amino Acid Sequence |
|---|---|---|---|
| Accession Nr. | Organism | SEQ ID NO: | Identity to I-CreI |
| P05725 | C. reinhardtii | 60 | 100 |
| Q8SMM1 | C. lunzensis | 61 | 56 |
| Q8SML7 | C. olivieri | 62 | 58 |
| Q1KVQ8 | S. obliquus | 63 | 49 |

TABLE 3
Exemplary homologs of PI-SceI, which can be
cloned from other organisms.

| Uni-Prot | | | Amino Acid Sequence |
|---|---|---|---|
| Accession Nr. | Organism | SEQ ID NO: | Identity to PI-SceI |
| P17255 | S. cerevisiae | 79 | 100 |
| Q874G9 | S. cerevisiae | 80 | 99 |
| Q874F9 | S. pastorianus | 81 | 97 |
| Q8J0H1 | S. cariocanus | 82 | 87 |
| Q8J0G4 | Z. bailii | 83 | 61 |
| Q8J0G5 | T. pretoriensis | 84 | 55 |

TABLE 4
Exemplary homologs of I-CeuI, which can be
cloned from other organisms.

| Uni-Prot | | SEQ ID | Amino Acid |
|---|---|---|---|
| Accession Nr. | Organism | NO: | Sequence Identity to I-CeuI |
| P32761 | C. moewusii | 65 | 100% |
| Q8WKZ1 | C. echinozygotum | 66 | 63% |
| Q8WL12 | C. elongatum | 67 | 58% |
| Q8WL11 | A. stipitatus | 68 | 55% |
| Q8WKX7 | C. monadina | 69 | 51% |

TABLE 5
Exemplary homologs of I-ChuI, which can be
cloned from other organisms.

| Uni-Prot | | | Amino Acid |
|---|---|---|---|
| Accession Nr. | Organism | SEQ ID NO: | Sequence Identity to I-CeuI |
| Q53X18 | C. humicola | 70 | 100% |
| Q8WL03 | C. zebra | 71 | 67% |
| Q8WKX6 | C. monadina | 72 | 62% |
| Q8WL10 | A. stipitatus | 73 | 58% |
| Q8SMI6 | N. aquatica | 74 | 54% |

TABLE 6
Exemplary homologs of I-DmoI, which can be
cloned from other organisms.

| Uni-Prot | | SEQ ID | Amino Acid Sequence |
|---|---|---|---|
| Accession Nr. | Organism | NO: | Identity to I-CeuI |
| P21505 | D. mobilis | 75 | 100% |
| Q6L6Z4 | Thermoproteus sp. | 76 | 51% |
| Q6L6Z5 | Thermoproteus sp. | 77 | 50% |
| A3MXB6 | P. calidifontis | 78 | 49% |

Homologs of endonucleases, which are cloned from other organisms might have a different enzymatic activity, DNA-binding-affinity, dimer formation affinity or changes in its DNA recognition sequence, when compared to the reference endonucleases, like I-SceI for homologs described in Table 1, I-CreI for homologs described in Table 2, or PI-SceI for homologs described in Table 3, or I-CeuI for homologs described in Table 4, or I-ChuI for homologs described in Table 5, or I-DmoI for homologs described in Table 6.

Preferred are LAGLIDADG endonucleases for which exact protein crystal structures have been determined, like I-Dmo I, H-Dre I, I-Sce I, I-Cre I, homologs of any one these having at least 49%, 51%, 58%, 60%, 70%, 80%, 85%, 90%, 92%, 93%, 94%, 95%, 96%, 97%, 98% or 99% sequence identity on amino acid level and which can easily be modeled on crystal structures of I-Dmo I, H-Dre I, I-Sce I, I-Cre I. One example, of an endonuclease, which can be modeled on the crystal structure of I-Cre I, is I-Mso I (SEQ ID NO: 84), (Chevalier et al., Flexible DNA Target Site Recognition by Divergent Homing Endonuclease Isoschizomers I-CreI and I-MsoI, J. Mol. Biol. (2003) 329, pages 253-269).

Another way to create homologs of LAGLIDADG endonucleases is to mutate the amino acid sequence of an LAGLIDADG endonuclease in order to modify its DNA binding affinity, its dimer formation affinity or to change its DNA recognition sequence. The determination of protein structure as well as sequence alignments of homologs of LAGLIDADG endonucleases allows for rational choices concerning the amino acids, that can be changed to affect its enzymatic activity, its DNA-binding-affinity, its dimer formation affinity or to change its DNA recognition sequence.

Homologs of LAGLIDADG endonucleases, which have been mutated in order to modify their DNA binding affinity, its dimer formation affinity or to change its DNA recognition site are called engineered endonucleases.

One approach to create engineered endonucleases is to employ molecular evolution. Polynucleotides encoding a candidate endonuclease enzyme can, for example, be modulated with DNA shuffling protocols. DNA shuffling is a process of recursive recombination and mutation, performed by random fragmentation of a pool of related genes, followed by reassembly of the fragments by a polymerase chain reaction-like process. See, e.g., Stemmer (1994) Proc Natl Acad Sci USA 91:10747-10751; Stemmer (1994) Nature 370:389-391; and U.S. Pat. No. 5,605,793, U.S. Pat. No. 5,837,458, U.S. Pat. No. 5,830,721 and U.S. Pat. No. 5,811,238. Engineered endonucleases can also be created by using rational design, based on further knowledge of the crystal structure of a given endonuclease see for example Fajardo-Sanchez et al., “Computer design of obligate heterodimer meganucleases allows efficient cutting of custom DNA sequences”, Nucleic Acids Research, 2008, Vol. 36, No. 7 2163-2173.

Numerous examples of engineered endonucleases, as well as their respective DNA recognition sites are known in the art and are disclosed for example in: WO 2005/105989, WO 2007/034262, WO 2007/047859, WO 2007/093918, WO 2008/093249, WO 2008/102198, WO 2008/152524, WO 2009/001159, WO 2009/059195, WO 2009/076292, WO 2009/114321, or WO 2009/134714, WO 10/001,189 all included herein by reference.

Engineered versions of I-SceI, I-CreI, I-MsoI and I-CeuI having an increased or decreased DNA-binding affinity are for example disclosed in WO07/047,859 and WO09/076,292. If not explicitly mentioned otherwise, all mutants will be named according to the amino acid numbers of the wildtype amino acid sequences of the respective endonuclease, e.g. the mutant L19 of I-SceI will have an amino acid exchange of leucine at position 19 of the wildtype I-SceI amino acid sequence, as described by SEQ ID NO: 1. The L19H mutant of I-SceI, will have a replacement of the amino acid leucine at position 19 of the wildtype I-SceI amino acid sequence with hystidine.

For example, the DNA-binding affinity of I-SceI can be increased by at least one modification corresponding to a substitution selected from the group consisting of:

(a) substitution of D201, L19, L80, L92, Y151, Y188, I191, Y199 or Y222 with H, N, Q, S, T, K or R; or
(b) substitution of N15, N17, S81, H84, N94, N120, T156, N157, S159, N163, Q165, S166, N194 or S202 with K or R.

DNA-binding affinity of I-SceI can be decreased by at least one mutation corresponding to a substitution selected from the group consisting of:

(a) substitution of K20, K23, K63, K122, K148, K153, K190, K193, K195 or K223 with H, N, Q, S, T, D or E; or
(b) substitution of L19, L80, L92, Y151, Y188, I191, Y199, Y222, N15, N17, S81, H84, N94, N120, T156, N157, S159, N163, Q165, S166, N194 or S202 with D or E.

Engineered versions of I-SceI, I-CreI, I-MsoI and I-CeuI having a changed DNA recognition sequence are disclosed in WO07/047,859 and WO09/076,292.

For example, an important DNA recognition site of I-SceI has the following sequence:

| | |
|---|---|
| sense: | 5′-T T A C C C T G T T A T C C C T A G-3′ |
| base position: | 1 2 3 4 5 6 7 8 9 10 11 12 13 14 15 16 17 18 |
| antisense | 3′-A A T G G G A C A A T A G G G A T C-5′ |

The following mutations of I-SceI will change the preference for C at position 4 to A: K50

The following mutations of I-SceI will keep the preference for C at position 4: K50, CE57

The following mutations of I-SceI will change the preference for C at position 4 to G: E50, R57, K57.

The following mutations of I-SceI will change the preference for C at position 4 to T: K57, M57, Q50.

The following mutations of I-SceI will change the preference for C at position 5 to A: K48, Q102. The following mutations of I-SceI will keep the preference for C at position 5: R48, K48, E102, E59

The following mutations of I-SceI will change the preference for C at position 5 to G: E48, K102, R102.

The following mutations of I-SceI will change the preference for C at position 5 to T: Q48, C102, L102, V102.

The following mutations of I-SceI will change the preference for C at position 6 to A: K59.

The following mutations of I-SceI will keep the preference for C at position 6: R59, K59.

The following mutations of I-SceI will change the preference for C at position 6 to G: K84, E59.

The following mutations of I-SceI will change the preference for C at position 6 to T: Q59, Y46.

The following mutations of I-SceI will change the preference for T at position 7 to A: C46, L46, V46.

The following mutations of I-SceI will change the preference for T at position 7 to C: R46, K46, E86.

The following mutations of I-SceI will change the preference for T at position 7 to G: K86, R86, E46.

The following mutations of I-SceI will keep the preference for T at position 7: K68, C86, L86, Q46*.

The following mutations of I-SceI will change the preference for G at position 8 to A: K61, S61, V61, A61, L61.

The following mutations of I-SceI will change the preference for G at position 8: E88, R61, H61.

The following mutations of I-SceI will keep the preference for G at position 8: E61, R88, K88.

The following mutations of I-SceI will change the preference for G at position 8 to T: K88, Q61, H61.

The following mutations of I-SceI will change the preference for T at position 9 to A: T98, C98, V98, L9B.

The following mutations of I-SceI will change the preference for T at position 9 to C: R98, K98.

The following mutations of I-SceI will change the preference for T at position 9 to G: E98, D98.

The following mutations of I-SceI will keep the preference for T at position 9: Q98.

The following mutations of I-SceI will change the preference for T at position 10 to A: V96, C96, A96.

The following mutations of I-SceI will change the preference for T at position 10 to C: K96, R96.

The following mutations of I-SceI will change the preference for T at position 10 to G: D96, E96.

The following mutations of I-SceI will keep the preference for T at position 10: Q96.

The following mutations of I-SceI will keep the preference for A at position 11: C90, L90.

The following mutations of I-SceI will change the preference for A at position 11 to C: K90, R90.

The following mutations of I-SceI will change the preference for A at position 11 to G: E90.

The following mutations of I-SceI will change the preference for A at position 11 to T: Q90.

The following mutations of I-SceI will change the preference for T at position 12 to A: Q193.

The following mutations of I-SceI will change the preference for T at position 12 to C: E165, E193, D193.

The following mutations of I-SceI will change the preference for T at position 12 to G: K165, R165.

The following mutations of I-SceI will keep the preference for T at position 12: C165, L165, C193, V193, A193, T193, S193.

The following mutations of I-SceI will change the preference for C at position 13 to A: C193, L193.

The following mutations of I-SceI will keep the preference for C at position 13: K193, R193, D192.

The following mutations of I-SceI will change the preference for C at position 13 to G: E193, D193, K163, R192.

The following mutations of I-SceI will change the preference for C at position 13 to T: Q193, C163, L163.

The following mutations of I-SceI will change the preference for C at position 14 to A: L192, C192.

The following mutations of I-SceI will keep the preference for C at position 14: E161, R192, K192.

The following mutations of I-SceI will change the preference for C at position 14 to G: K147, K161, R161, R197, D192, E192.

The following mutations of I-SceI will change the preference for C at position 14 to T: K161, Q192.

The following mutations of I-SceI will change the preference for C at position 15 to A: none identified.

The following mutations of I-SceI will keep the preference for C at position 15: E151.

The following mutations of I-SceI will change the preference for C at position 15 to G: K151.

The following mutations of I-SceI will change the preference for C at position 15 to T: C151, L151, K151.

The following mutations of I-SceI will keep the preference for A at position 17: N152, S152, C150, L150, V150, T150.

The following mutations of I-SceI will change the preference for A at position 17 to C: K152, K150.

The following mutations of I-SceI will change the preference for A at position 17 to G: N152, S152, D152, D150, E150.

The following mutations of I-SceI will change the preference for A at position 17 to T: Q152, Q150.

The following mutations of I-SceI will change the preference for G at position 18 to A: K155, C155.

The following mutations of I-SceI will change the preference for G at position 18: R155, K155.

The following mutations of I-SceI will keep the preference for G at position 18: E155.

The following mutations of I-SceI will change the preference for G at position 18 to T: H155, Y155.

Combinations of several mutations may enhance the effect. One example is the triple mutant W149G, D150C and N152K, which will change the preference of I-SceI for A at position 17 to G.

In order to preserve the enzymatic activity of the LAGLIDADG endonucleases the following mutations should be avoided:

For I-Sce I: I38S, I38N, G39D, G39R, L40Q, L42R, D44E, D44G, D44H, D44S, A45E, A45D, Y46D, I47R, I47N, D144E, D145E, D145N and G146E.

### for I-CreI: Q47E,

### for I-CeuI E66Q,

### for I-MsoI D22N,

for PI-SceI mutations in D218, D229, D326 or T341.

Engineered endonuclease variants of I-AniI having high enzymatic activity can be found in Takeuchi et al., Nucleic Acid Res. (2009), 73(3): 877 to 890. Preferred engineered endonuclease variants of I-Ani I, as described by SEQ ID NO: 142, comprise the following mutations: F13Y and S111Y, or F13Y, S111Y and K222R, or F13Y, 155V, F91I, S92T and S111Y.

Mutations which alter the DNA-binding-affinity, the dimer formation affinity or change the DNA recognition sequence of a given endonuclease, e.g. a LAGLIDADG endonuclease, may be combined to create an engineered endonuclease, e.g. an engineered endonuclease based on I-SceI and having an altered DNA-binding-affinity and/or a changed DNA recognition sequence, when compared to I-SceI as described by SEQ ID NO: 1.

### Optimized Nucleases:

Nucleases can be optimized for example by inserting mutations to change their DNA binding specificity, e.g to make their DNA recognition site more or less specific, or by adapting the polynucleotide sequence coding for the nuclease to the codon usage of the organism, in which the endonuclease is intended to be expressed, or by deleting alternative start codons, or by deleting cryptic polyadenylation signals from the polynucleotide sequence coding for the endonuclease.

Mutations and changes in order to create optimized nucleases may be combined with the mutations used to create engineered endonucleases, for example, a homologue of I-SceI may be an optimized nuclease as described herein, but may also comprise mutations used to alter its DNA-binding-affinity and/or change its DNA recognition sequence.

Further optimization of nucleases may enhance protein stability. Accordingly optimized nucleases do not comprise, or have a reduced number compared to the amino acid sequence of the non optimized nuclease of:

### a) PEST-Sequences,

### b) KEN-boxes

### c) A-boxes,

### d) D-boxes, or

e) comprise an optimized N-terminal end for stability according to the N-end rule,
f) comprise a glycin as the second N-terminal amino acid, or
g) any combination of a), b), c) d), e) and f).

PEST Sequences are required to contain at least one proline (P), one aspartate (D) or glutamate (E) and at least one serine (S) or threonine (T). Negatively charged amino acids are clustered within these motifs while positively charged amino acids, arginine (R), histidine (H) and lysine (K) are generally forbidden. PEST Sequences are for example described in Rechsteiner M, Rogers S W. “PEST sequences and regulation by proteolysis.” Trends Biochem. Sci. 1996; 21(7), pages 267 to 271.

The amino acid consensus sequence of a KEN-box is: KENXXX(N/D)

The amino acid consensus sequence of a A-box is: AQRXLXXSXXXQRVL

The amino acid consensus sequence of a D-box is: RXXL

A further way to stabilize nucleases against degradation is to optimize the amino acid sequence of the N-terminus of the respective endonuclease according to the N-end rule. Nucleases which are optimized for the expression in eucaryotes comprise either methionine, valine, glycine, threonine, serine, alanine or cysteine after the start methionine of their amino acid sequence. Nucleases which are optimized for the expression in procaryotes comprise either methionine, valine, glycine, threonine, serine, alanine, cysteine, glutamic acid, glutamine, aspartic acid, asparagine, isoleucine or histidine after the start methionine of their amino acid sequence.

Nucleases may further be optimized by deleting 50, 40, 30, 20, 10, 9, 8, 7, 6, 5, 4, 3, 2, or 1 amino acids of its amino acid sequence, without destroying its endonuclease activity. For example, in case parts of the amino acid sequence of a LAGLIDADG endonuclease is deleted, it is important to retain the LAGLIDADG endonuclease motif described above.

It is preferred to delete PEST sequences or other destabilizing motifs like KEN-box, D-box and A-box. Those motifs can also be destroyed by introduction of single amino acid exchanges, e.g introduction of a positively charged aminoacid (arginine, histidine and lysine) into the PEST sequence.

Another way to optimize nucleases is to add nuclear localization signals to the amino acid sequence of the nuclease. For example a nuclear localization signal as described by SEQ ID NO: 4.

Optimized nucleases may comprise a combination of the methods and features described above, e.g. they may comprise a nuclear localization signal, comprise a glycine as the second N-terminal amino acid or a deletion at the C-terminus or a combination of these features. Examples of optimized nucleases having a combination of the methods and features described above are for example described by SEQ ID NOs: 2, 3 and 5.

In one embodiment the optimized nuclease is an optimized I-Sce-I, which does not comprise an amino acid sequence described by the sequence: HVCLLYDQWVLSPPH, LAYWFMDDGGK, KTIPNNLVENYLTPMSLAYWFMDDGGK, KPIIYIDSMSYLIFYNLIK, KLPNTISSETFLK or TISSETFLK,

or which does not comprise an amino acid sequence described by the sequence: HVCLLYDQWVLSPPH, LAYWFMDDGGK, KPIIYIDSMSYLIFYNLIK, KLPNTISSETFLK or TISSETFLK,
or which does not comprise an amino acid sequence described by the sequence: HVCLLYDQWVLSPPH, LAYWFMDDGGK, KLPNTISSETFLK or TISSETFLK,
or which does not comprise an amino acid sequence described by the sequence: LAYWFMDDGGK, KLPNTISSETFLK or TISSETFLK,
or which does not comprise an amino acid sequence described by the sequence: KLPNTISSETFLK or TISSETFLK,

In one embodiment the optimized nuclease is I-SceI, or its homologs having at least 49%, 51%, 58%, 60%, 70%, 80%, 85%, 90%, 92%, 93%, 94%, 95%, 96%, 97%, 98% or 99% sequence identity on amino acid level in which the amino acid sequence TISSETFLK at the C-terminus of wildtype I-SceI or its homologs having at least 49%, 51%, 58%, 60%, 70%, 80%, 85%, 90%, 92%, 93%, 94%, 95%, 96%, 97%, 98% or 99% sequence identity on amino acid level and having an amino acid sequence TISSETFLK at the C-terminus, is deleted or mutated.

The amino acid sequence TISSETFLK may be deleted or mutated, by deleting or mutating at least 1, 2, 3, 4, 5, 6. 7, 8 or 9 amino acids of the C-terminus of wildtype I-SceI or its homologs having at least 49%, 51%, 58%, 60%, 70%, 80%, 85%, 90%, 92%, 93%, 94%, 95%, 96%, 97%, 98% or 99% sequence identity on amino acid level and having an amino acid sequence TISSETFLK at the C-terminus.

TABLE 7
Different examples for deletions of the TISSETFLK
amino acid sequence in wildtype I- SceI

| Wildtype and | Amino Acid Sequence |
|---|---|
| optimized I-SceI | on C-terminus |
| I-SceI wildtype | TISSETFLK |
| I-SceI -1 | TISSETFL |
| I-SceI -2 | TISSETF |
| I-SceI -3 | TISSET |
| I-SceI -4 | TISSE |
| I-SceI -5 | TISS |
| I-SceI -6 | TIS |
| I-SceI -7 | TI |
| I-SceI -8 | T |
| I-SceI -9 | all 9 amino acids on C-terminus |
| | of wt I-SceI deleted |

Alternatively the amino acid sequence TISSETFLK may be mutated, e.g. to the amino acid sequence: TIKSETFLK (SEQ ID NO: 149), or AIANQAFLK (SEQ ID NO: 150).

Equally preferred, is to mutate serine at position 229 of the amino acid sequence of wildtype I-SceI as disclosed in SEQ ID No. 1 (being amino acid 230 if referenced to SEQ ID No. 2) to Lys, Ala, Pro, Gly, Glu, Gln, Asp, Asn, Cys, Tyr or Thr. Thereby creating the I-SceI mutants S229K, S229A, S229P, S229G, S229E, S229Q, S229D, S229N, S229C, S229Y, or S229T (amino acids are numbered according to SEQ ID No. 1.

In another embodiment of the invention, the amino acid methionine at position 203 of the amino acid sequence of wildtype I-SceI as disclosed in SEQ ID No. 1 (being amino acid 204 if referenced to SEQ ID No. 2), is mutated to Lys, His or Arg. Thereby creating the I-SceI mutant M203K, M203H and M203R.

Preferred optimized versions of I-SceI are the deletions I-SceI-1, I-SceI-2, I-SceI-3, I-SceI-4, I-SceI-5, I-SceI-6, I-SceI-7, I-SceI-8, I-SceI-9 and the mutants S229K and S229H, S229R even more preferred are the deletions I-SceI-1, I-SceI-2, I-SceI-3, I-SceI-4, I-SceI-5, I-SceI-6 and the mutant S229K.

It is also possible to combine the deletions and mutations described above, e.g. by combining the deletion I-SceI-1 with the mutant S229K, thereby creating the amino acid sequence TIKSETFL at the C-terminus.

It is also possible to combine the deletions and mutations described above, e.g. by combining the deletion I-SceI-1 with the mutant S229A, thereby creating the amino acid sequence TIASETFL at the C-terminus.

Further preferred optimized versions of I-SceI are the deletions I-SceI-1, I-SceI-2, I-SceI-3, I-SceI-4, I-SceI-5, I-SceI-6, I-SceI-7, I-SceI-8, I-SceI-9 or the mutants S229K and S229H, S229R, in combination with the mutation M203K, M203H, M203R.

Even more preferred are the deletions I-SceI-1, I-SceI-2, I-SceI-3, I-SceI-4, I-SceI-5, I-SceI-6 or the mutant S229K in combination with the mutation M203K.

In another embodiment of the invention, the amino acids glutamine at position 75, glutamic acid at position 130, or tyrosine at position 199 of the amino acid sequence of wildtype I-SceI as disclosed in SEQ ID No. 1 (being amino acids 76, 131 and 120 if referenced to SEQ ID No. 2), are mutated to Lys, His or Arg. Thereby creating the I-SceI mutants Q75K, Q75H, Q75R, E130K, E130H, E130R, Y199K, Y199H and Y199R.

The deletions and mutations described above will also be applicable to its homologs of I-SceI having at least 49%, 51%, 58%, 60%, 70%, 80%, 85%, 90%, 92%, 93%, 94%, 95%, 96%, 97%, 98% or 99% sequence identity on amino acid level and having an amino acid sequence TISSETFLK at the C-terminus.

Accordingly, in one embodiment of the invention, the optimized endonuclease, is an optimized version of I-SceI or one of its homologs having at least 49%, 51%, 58%, 60%, 70%, 80%, 85%, 90%, 92%, 93%, 94%, 95%, 96%, 97%, 98% or 99% sequence identity on amino acid level, and having one or more of the mutations or deletions selected from the group of: I-SceI-1, I-SceI-2, I-SceI-3, I-SceI-4, I-SceI-5, I-SceI-6, I-SceI-7, I-SceI-8, I-SceI-9, S229K, S229A, S229P, S229G, S229E, S229Q, S229D, S229N, S229C, S229Y, S229T, M203K, M203H, M203R, Q77K, Q77H, Q77R, E130K, E130H, E130R, Y199K, Y199H and Y199R, wherein the amino acid numbers are referenced to the amino acid sequence as described by SEQ ID NO: 1.

In a further embodiment of the invention, the optimized endonuclease, is an optimized version of I-SceI or one of its homologs having at least 49%, 51%, 58%, 60%, 70%, 80%, 85%, 90%, 92%, 93%, 94%, 95%, 96%, 97%, 98% or 99% sequence identity on amino acid level, and having one or more of the mutations or deletions selected from the group of: I-SceI-1, I-SceI-2, I-SceI-3, I-SceI-4, I-SceI-5, I-SceI-6, S229K and M203K, wherein the amino acid numbers are referenced to the amino acid sequence as described by SEQ ID NO: 1.

A particular preferred optimized endonuclease is a wildtype or engineered version of I-SceI, as described by SEQ ID NO: 1 or one of its homologs having at least 49%, 51%, 58%, 60%, 70%, 80%, 85%, 90%, 92%, 93%, 94%, 95%, 96%, 97%, 98% or 99% sequence identity on amino acid level and having one or more mutations selected from the groups of:

### a) I-SceI-1, I-SceI-2, I-SceI-3, I-SceI-4, I-SceI-5, I-SceI-6, I-SceI-7, I-SceI-8 and I-SceI-9;

### b) S229K, S229A, S229P, S229G, S229E, S229Q, S229D, S229N, S229C, S229Y, S229T, M203K, M203H, M203R, Q77K, Q77H, Q77R, E130K, E130H, E130R, Y199K, Y199H and Y199R;

c) a methionine, valine, glycine, threonine, serine, alanine, cysteine, glutamic acid, glutamine, aspartic acid, asparagine, isoleucine or histidine after the start methionine of their amino acid sequence; or
d) a combination of one or more mutations selected from a) and b), a) and c), b) and c) or a) b) and c) above.

### Heterologous DNA Binding Domains:

The chimeric endonuclease of the invention comprises at least one heterologous DNA binding domain.

Heterologous DNA binding domains are polypeptides binding to polynucleotides having a specific polynucleotide sequence (recognition sequence or operator sequence). Examples for heterologous DNA binding domains are eukaryotic, prokaryotic or viral transcription factors. In one embodiment of the invention, only the DNA binding domain of the eukaryotic, prokaryotic or viral transcription factor is used as heterologous DNA binding domain.

Preferrably heterologous DNA binding domains are selected from eukaryotic, prokaryotic and viral transcription factors or their respective DNA binding domains, which bind DNA as monomers or single chain variants, which bind their DNA recognition sequence with high affinity and specificity, and have an N- or C-Terminus on the surface of the protein.

Especially preferred are eukaryotic, prokaryotic and viral transcription factors or their respective DNA binding domains of which the three dimensional structure of at least a homolog of the respective eukaryotic, prokaryotic and viral transcription factors or their respective DNA binding domain has been determined.

The term heterologous DNA binding domain shall not comprise more than two repetitions of modular C₂H₂ zink finger domains, as disclosed for example in WO07/014,275, WO08/076,290, WO08/076,290 or WO03/062455. C₂H₂ Zinc finger domains have conserved cysteine and histidine residues that tetrahedycally-coordinate the single zinc atom in each finger domain and are characterized by finger components having the general sequence: -Cys-(X)₂₋₄-Cys-(X)₁₂-His-(X)₃₋₅-His- in which X represents any amino acid. (the C₂H₂ ZFPs).

Numerous eukaryotic, prokaryotic and viral transcription factors as well as their respective recognition sequences or operator sequences have been described in the art. Information on eukaryotic, prokaryotic and viral transcription factors as well as their respective recognition sequences as well as numerous three dimensional structures can be found in public available databases and bioinformatic analysis tools, for example in:
JASPAR 2010 (Partales-Casamar et al. (2009), Nucl. Acids Res., 1 to 6),
UniPROBE (Newburger, D. E. and Bulyk, M. L. (2008), Nucl. Acids Res., 37, Database issue, D77 to D82),
PLACE (Higo et al. (1999), Nucl. Acids Res., 27 (1), 297 to 300).
RegTransBase (Kazakov, A. E., et al. (2007) Nucleic acids research 35, D407 to 412)
RegulonDB (Gama-Castro, S., et al. (2008) Nucleic acids research 36, D120 to 124)
DP Interact (Robison, K., et al. (1998) J Mol Biol 284, 241 to 254)
FlyReg (Bergman, C. M., et al. (2005) Bioinformatics 21, 1747 to 1749)
Zhu, C., et al. (2009), Genome Res 19, 556 to 566
Harbison, C. T., et al. (2004), Nature 431, 99 to 104
Maclsaac, K. D., et al. (2006) BMC bioinformatics 7, 113

The DNA binding domain database (DBD) (http://transcriptionfactor.org) includes predictions of sequence specific transcription factors of over 700 species (Teichmann (2007) Nucleic Acids Research 36:D88-D92).

Preferred heterologous DNA binding domains are proteins with known binding properties and recognition sequences; more preferable proteins which have been co-cristalized with their specific DNA target.

Eukaryotic, prokaryotic and viral transcription factors have been grouped in several protein families, having an individual PF-Number as identifier.

Heterologous DNA-binding domains can for example be found in the following protein families:

PF00126 Bacterial regulatory helix-turn-helix protein, lysR family
PF00486 Transcriptional regulatory protein, C terminal
PF04383 KiIA-N domain

### PF01381 Helix-turn-helix

PF02954 Bacterial regulatory protein, F is family
PF00313 Cold-shock DNA-binding domain
PF00325 Bacterial regulatory proteins, crp family
PF01047 MarR family
PF04299 Putative FMN-binding domain
PF00392 Bacterial regulatory proteins, gntR family
PF00165 Bacterial regulatory helix-turn-helix proteins, AraC family
PF05225 helix-turn-helix, Psq domain
PF00847 AP2 domain
PF04967 HTH DNA binding domain
PF08279 HTH domain
PF01022 Bacterial regulatory protein, arsR family
PF00196 Bacterial regulatory proteins, luxR family
PF00010 Helix-loop-helix DNA-binding domain
PF00356 Bacterial regulatory proteins, lacI family
PF02082 Transcriptional regulator
PF00292 Paired box domain
PF04397 LytTr DNA-binding domain
PF03749 Sugar fermentation stimulation protein
PF04353 Regulator of RNA polymerase sigma70 subunit, Rsd/AlgQ

Preferably heterologous DNA binding domains are selected from members of the following protein families:

PF00126 Bacterial regulatory helix-turn-helix protein, lysR family
PF00165 Bacterial regulatory helix-turn-helix proteins, AraC family
PF01022 Bacterial regulatory protein, arsR family
PF00196 Bacterial regulatory proteins, luxR family
PF00010 Helix-loop-helix DNA-binding domain
PF00356 Bacterial regulatory proteins, lacI family

Even more preferred are members of the following protein families:

PF00126 Bacterial regulatory helix-turn-helix protein, lysR family
PF00165 Bacterial regulatory helix-turn-helix proteins, AraC family
PF00196 Bacterial regulatory proteins, luxR family
PF00356 Bacterial regulatory proteins, lacI family

A particular preferred group of heterologous DNA binding domains are proteins comprising a helix-turn-helix DNA binding domain (HTH domain). Such proteins are for example scTetR, ArcR and proteins of the Lad, AraC and MerR protein families.

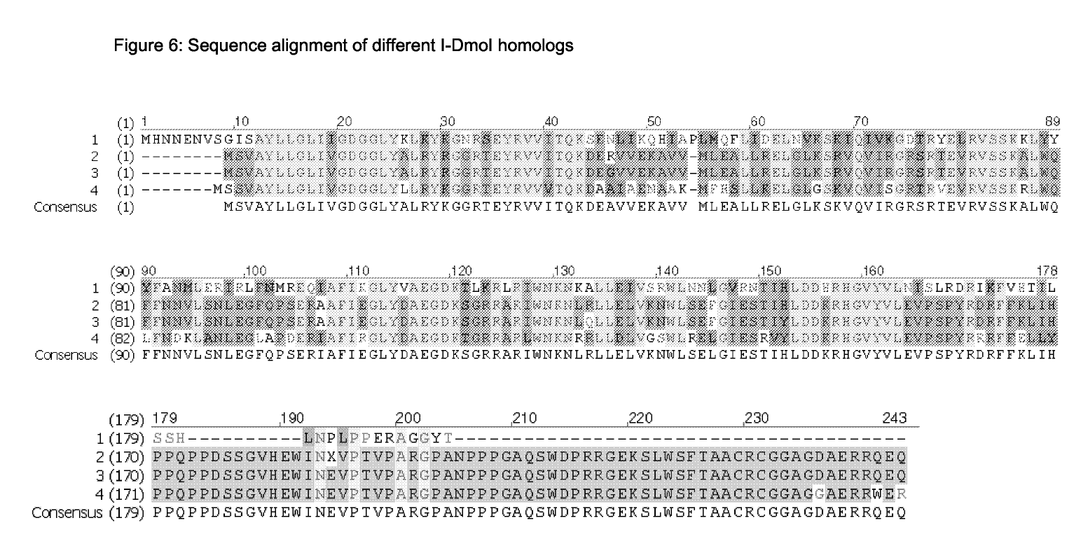

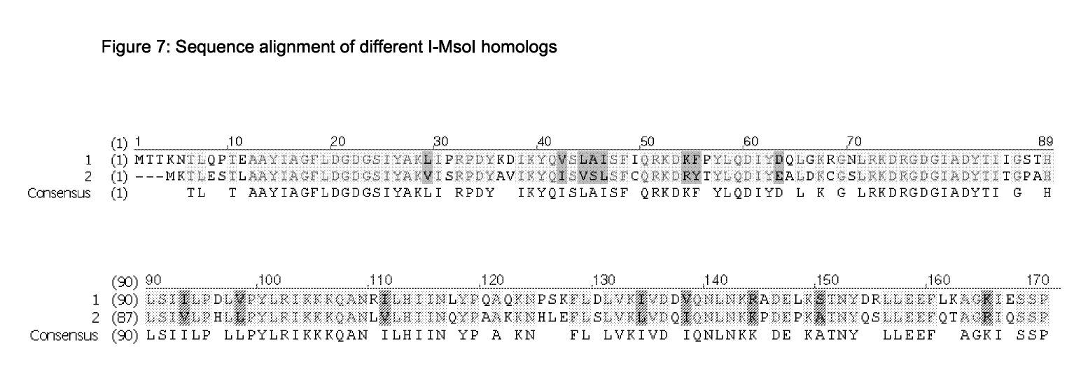

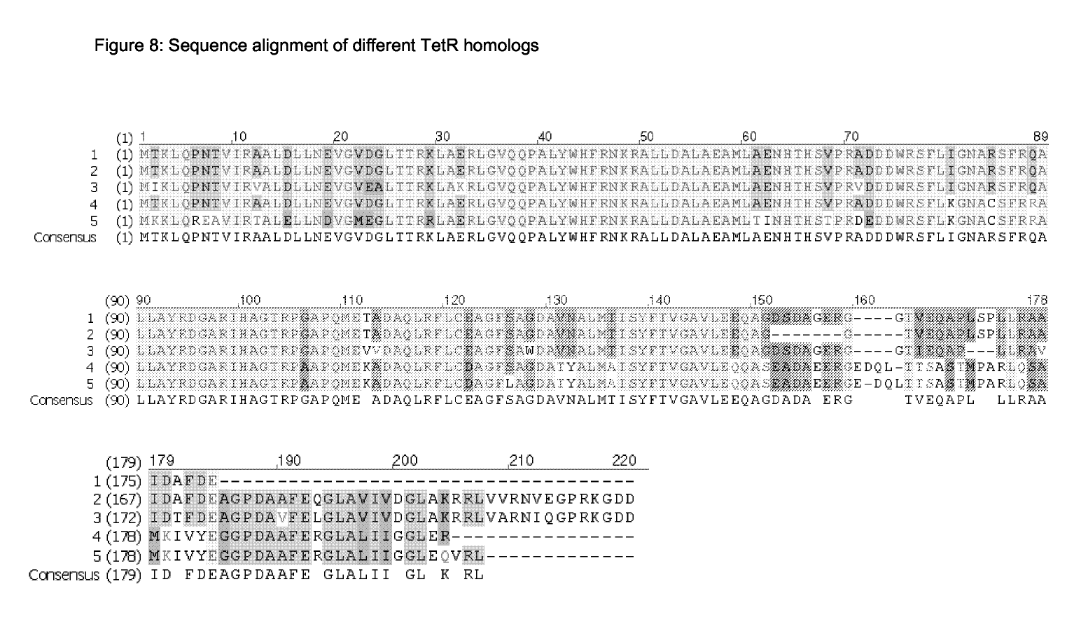

Information about the TetR (scTetR) protein family can be found in: Ramos J. L. et al. “The RetR Family of Transcriptional Repressors”, Microbiology and Molecular Biology Reviews (2005), pages 326 to 356 and Ralph Bertram et al., “The application of Tet repressor in prokaryotic gene regulation and expression.”, (2008) Microbial Biotechnology, 1(1), pages 2-16 and Marcus Krueger et al., “Engineered Tet repressors with recognition specificity for the tetO-4C5G operator variant”, (2007), Gene, 404, pages 93-100 and Xue Zhou et al., “Improved single-chain transactivators of the Tet-On gene expression system”, (2007), BMC Biotechnology, 7:6. Examples and common features of proteins belonging to the TetR protein family are given by SEQ ID NO: 86, 87, 88, 89 and 90 and the alignment shown in FIG. 8, Examples and common features of the respective HTH domains are given by SEQ ID NO: 91, 92, 93, 94 and 95 and the alignment shown in FIG. 9a.

Information about the LacI (Lac Repressor or Lac Inhibitor) protein family can be found in: Weickert J. M. and Adhya S., “A Family of Bacterial Regulators Homologous to Gal and Lac Repressors”, The Journal ov Biological Chemistry, Vol. 267, pages 15869 to 15874 and Liskin Swint-Kruse et al., “Allostery in the LacI/GalR family: variations on a theme”, (2009), Current Opinion in Microbiology, 12:129-137 and Catherine M. Falcon et al., “Operator DNA Sequence Variation Enhances High Affinity Binding by Hinge Helix Mutants of Lactose Repressor Protein”, (2000), Biochemistry, 39, 11074-11083 and Christof Francke et al., “A generic approach to identify Transcription Factor-specific operator motifs; Inferences for LacI-family mediated regulation in Lactobacillus plantarum WCFS1”, (2008), BMC Genomics, 9:145.

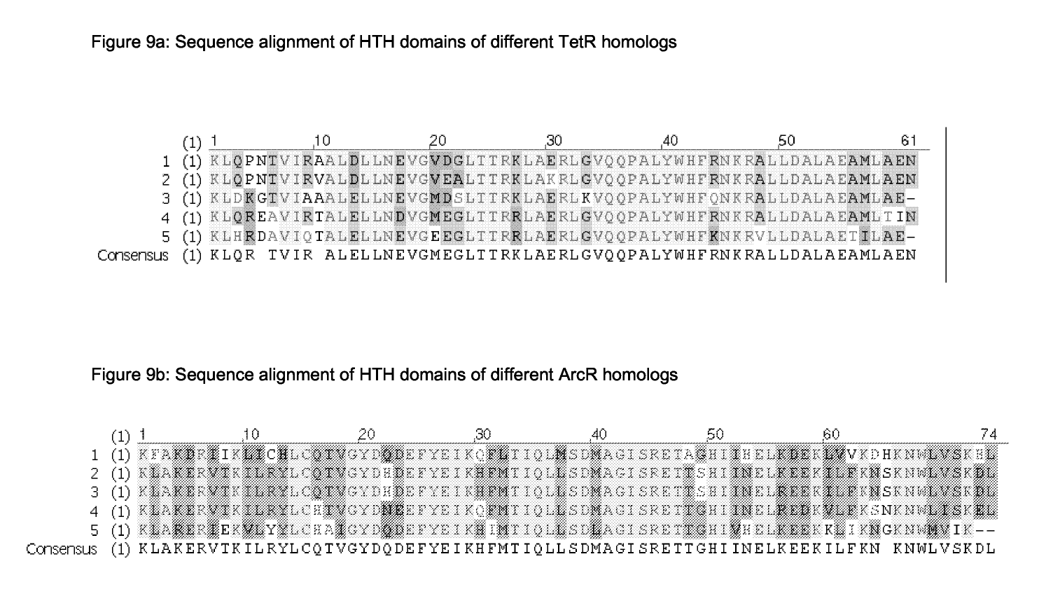

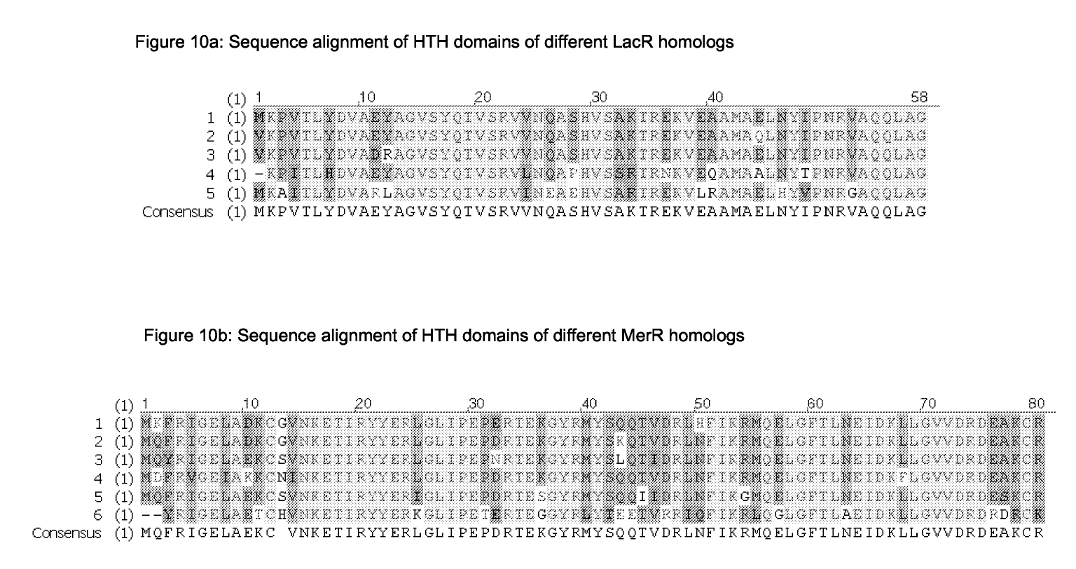

Examples and common features of the HTH domains of proteins belonging to the Lac Repressor protein family are given by SEQ ID NO: 101, 102, 103, 104 and 105 and the alignment shown in FIG. 10a.

Members of the AraC protein family and information about common features of these proteins are for example described in: Martin, R. Rosner, “The AraC transcriptional activators”, Current Opinion in Microbiology (2001), Vol. 4, pages 132 to 137. Members of the AraC protein family having two HTH domains are for examoly homologs of the MarA protein. Information about MarA and related proteins can be found in: Sangkee Rhee et al., “A novel DNA-binding motiv in MarA: The first structure for an AraC family transcriptional activator”, PNAS (1998), Vol. 95, pages 10413 to 10418 and in Gillette W. K. et al., “Probing the Escherichia coli Transcriptional Activator MarA using Alanine-scanning Mutagenesis: Residues Important for DNA Binding and Activation”, JMB (2000), Vol. 299, pages 1245 to 1255.

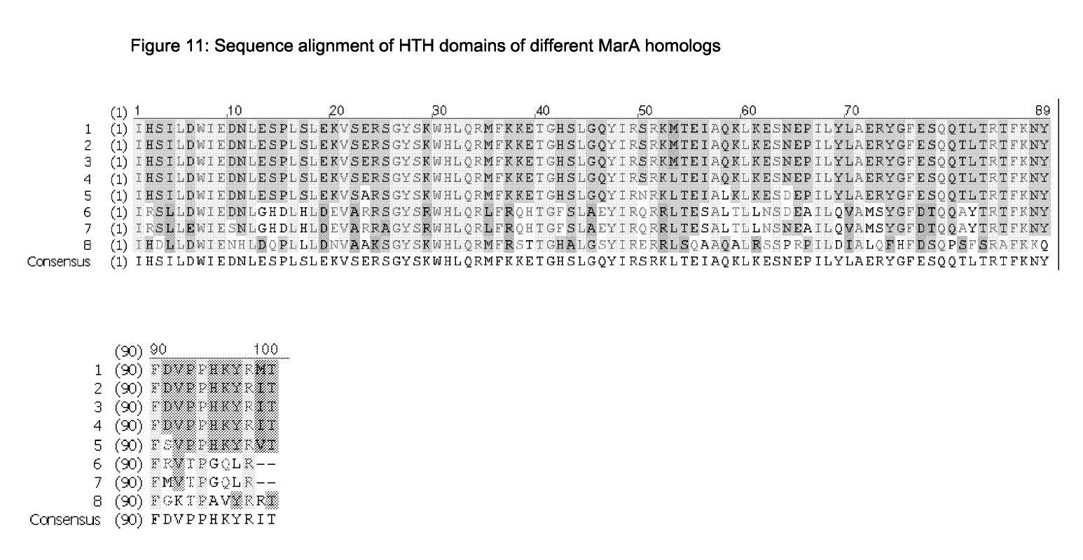

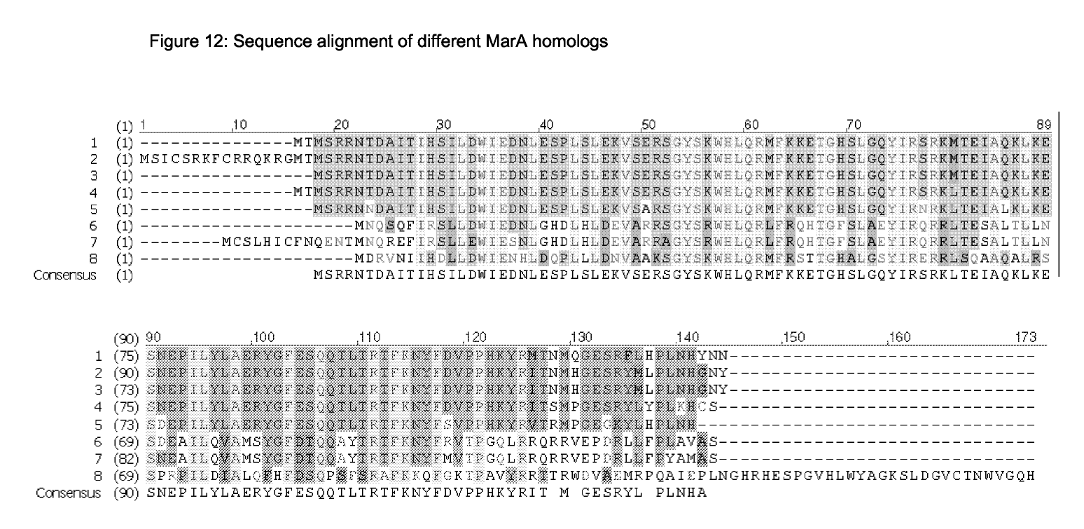

Examples and common features of proteins belonging to the AraC protein family in particular homologs of MarA are given by SEQ ID NO: 120, 121, 122, 123, 124, 125, 126 and 127 and the alignment shown in FIG. 12. Examples and common features of the HTH domains of proteins belonging to the AraC protein family in particular homologs of MarA are given by SEQ ID NO: 112, 113, 114, 115, 116, 117, 118 and 119 and the alignment shown in FIG. 11.

Information about the MerR protein family and common features of their HTH domain can be found in: Brown N. L. et al. “The MerR family of transcriptional regulators” FEMS Microbiology Reviews (2003), Vol. 27, pages 145 to 163. Examples and common features of the HTH domains of proteins belonging to the MerR protein family are given by SEQ ID NO: 106, 107, 108, 109, 110 and 111 and the alignment shown in FIG. 10b.

Proteins similar to the scArcR protein as described by SEQ ID NO: 7 comprise a HTH domain for DNA binding, different examples and common features of these HTH domains are given by SEQ ID NO: 96, 97, 98, 99 and 100 and the alignment shown in FIG. 9b.

Members of the WRKY protein family and information about common features of these proteins are for example described in: Eulgem, T. et al. “The WRKY superfamily of plant transcription factors.” (2000) Trends Plant Sci., 5, pages 199 to 206 and Ming-Rui Duan et al. “DNA binding mechanism revealed by high resolution crystal structure of Arabidopsis thaliana WRKY1 protein” (2007), Nucleic Acids Research, Vol. 35, No. 4 1145-1154, which are included herein by reference in their entirety.

Other suitable heterologous DNA binding domains are inactive endonucleases. Such endonucleases may be inactive in the target organism because they act only under certain, usually more extreme conditions (for example, high temperature). Alternatively, one may use a mutated endonuclease, whereas said mutation renders the endonuclease inactive. Inactive endonucleases are for example, but not excluding others: I-DmoI or other termophylic endonucleases employed at temperatures below 40° C., more preferable below 30° C., even more preferably below 25° C., and endonucleases having amino acid substitutions in their active center(s), for example I-CreI having the mutation of Q47 to E, I-Sce I having the mutation of D44 or D145 to N, I-CeuI having the mutation of E66 to Q, or I-MsoI having the mutation of D22 to N. A preferred inactive endonuclease is I-Sce I having the mutation of D44 to S (I-SceI^{D44S}). For example the following amino acid residues of PI-SceI: D218, D229, D326 and T341 Pingoud (2000) Biochemistry 39:15895-15900

In one embodiment at least one heterologous DNA binding domain is an inactive I-SceI, I-CreI, I-CeuI, I-ChuI, I-DmoI, Pi-SceI, I-MsoI, or I-AniI or an inactive homolog of these having at least 45%, 56%, 57%, 58%, 59%, 60%, 61%, 62%, 63%, 65%, 66%, 67%, 68%, 69%, 70%, 71%, 72%, 73%, 74%, 75%, 76%, 77%, 78%, 79%, 80%, 81%, 82%, 83%, 84%, 85%, 90%, 91%, 92%, 93%, 94%, 95%, 96%, 97%, 98%, 99% amino acid sequence identity. In one embodiment the heterologous DNA binding domain is an inactive version of a LAGLIDADG endonucleases having an amino acid sequence as described by at least one of SEQ ID NO: 1, 2, 3, 5, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 142 or 159, preferably having an amino acid sequence as described by any one of SEQ ID NO: 1, 2, 3, 5 or 159.

In one preferred embodiment the chimeric endonuclease comprises I-SceI or an optimized version of I-SceI and an heterologous DNA binding domain comprising an inactive I-SceI or an inactive version of an optimized version of I-SceI.

In one embodiment of the invention the term heterologous DNA binding domain does not comprise inactive endonucleases.

The heterologous DNA binding domain can comprise the full protein of a given transcription factor or a large fragment thereof or might only comprise a fragment more or less limited to the DNA binding domain of a transcription factor.

Examples for suitable transcription factors are for example, but not excluding others: scTet, scArcR, LacR, TraR, Gal, LambaR, LuxR, WRKY and homologs of any one these having at least 50%, 60%, 70%, 80%, 85%, 90%, 92%, 93%, 94%, 95%, 96%, 97%, 98% or 99% of sequence identity on amino acid level.

In a preferred embodiment the DNA binding activity of the heterologous DNA binding domain is inducible or repressible via binding of an Inductor to at least one of the DNA binding domains. The Inductor can be a polypeptide or a small organic substance.

Examples for inducible or repressible or inducible and repressible heterologous DNA binding domains and their inductors or repressors are:

scTet Tetracycline and Anhydrotetracycline and other derivates

### LacR, Lactose and IPTG

TraR, 30C8HL (N-(3-oxo)-octanoly-L-homoserine lactone)
LuxR family. acetylated homoserine lactones (AHL)
LuxR 3OC6HL (N-(3-oxo)-hexa-L-homoserine lactone)
LasR 30C12HL (N-(3-oxo)-duodeca-L-homoserine lactone)

### AraC Arabinose

### RhaR Rhamnose

MerR mercury ions

Preferably the heterologous DNA binding domain has a recognition sequence of at least 4, at least 6, at least 8, at least 10 or at least 12 base pairs.

Examples of recognition sequences of heterologous DNA binding domains are:

scTet
(SEQ ID NO: 130)
5′-YTATCATTGATAG-3′
TetR (only one monomer)
5′-YTATC-3′
scArcR (dimer or single chain variants)
(SEQ ID NO: 7)
5′-AATGATAGAAGCACTCTACTAT-3′
TraR (dimer or single chain variants)
(SEQ ID NO: 131)
5′-ATGTGCAGATCTGCACAT-3′
WRKY (dimer or single chain variants)
5′-YTGACY-3′
LacR (dimer or single chain variants)
5′-TTGTGAGC-3′
MarA (monomer)
(SEQ ID NO: 137)
5′-AYNGCACNNWNNRYYAAAYN-3′
MerR (monomer)
5′-TTKACY-3′,
MerR (dimer or single chain variant)
(SEQ ID NO: 138)
5′-TTKACYNNNNNNNNNNNNNNNNNNNTAAGGT-3′

wherein A stands for adenine, G for guanine, C for cytosine, T for thymine, R for guanine or adenine, Y for thymine or cytosine, K for guanine or thymine, W for adenine or thymine and n for adenine or guanine or cytosine or thymine

The person skilled in the art will acknowledge that most DNA binding domains will not be limited to bind only the exact recognition sequence, but also similar recognition sequences for example.

Examples for alternative recognition sequences of
LacR dimmers are
(SEQ ID NO: 132)
5′-TGTTTGATATCATATAAACA-3′
and
(SEQ ID NO: 133)
5′-GAATTGTGAGCGGATAACAATTT-3′
and
(SEQ ID NO: 134)
5′-GAATGTGAGCGAGTAACAACCG-3′
and
(SEQ ID NO: 135)
5′-CGGCAGTGAGCGCAACGCAATT-3′
and
(SEQ ID NO: 136)
5′-GAATTGTAAGCGCTTACAATT-3′

Preferred heterologous DNA binding domains are monomeric DNA binding domains e.g. HTH domains of transcription factors or monomeric transcription factors.

Similar preferred are DNA binding domains having a high specificity for one or a small group of recognition sequences.

Equally preferred are DNA binding domains having a high affinity for one or a small group of recognition sequences.

In one embodiment the heterologous DNA-binding domain comprises at least one HTH domain of scTet, scArcR, TraR, LacR, LuxR, MarA, or MerR and homologs of any one these having at least 50%, 60%, 70%, 80%, 85%, 90%, 92%, at 93%, 94%, 95%, 96%, 97%, 98% or 99% of sequence identity on amino acid level.

In a further embodiment of the invention, the transcription factor or the DNA binding domain of a transcription factor comprises a HTH domain comprising an amino acid sequence of at least 80%, 81%, 82%, 83%, 84%, 85%, 86%, 87%, 88%, 89%, 90%, 91%, 92%, 93%, 94%, 95%, 96%, 97%, 98%, 99% sequence identity to at least one amino acid sequence described by SEQ ID NO: 91, 92, 93, 94, 95, 96, 97, 98, 99, 100, 101, 102, 103, 104, 105, 106, 107, 108, 109, 110, 111, 112, 113, 114, 115, 116, 117, 118 or 119, preferably to at least one amino acid sequence described by 91, 92, 93, 94, 95, 112, 113, 114, 115, 116, 117, 118 or 119.

In another embodiment of the invention, the heterologous DNA-binding domain comprises a HTH domain having a sequence identity of at least 50%, 60%, 70%, 80%, 85%, 90%, 92%, at 93%, 94%, 95%, 96%, 97%, 98% or 99% of sequence identity on amino acid level to any one of SEQ ID NO: 91, 92, 93, 94, 95, 96, 97, 98, 99, 100, 101, 102, 103, 104, 105, 106, 107, 108, 109, 110, 111, 112, 113, 114, 115, 116, 117, 118 or 119.

In one embodiment the heterologous DNA-binding domain is selected from the group consisting of: scTet, scArcR, TraR, LacR, LuxR, MarA, or MerR and homologs of any one these having at least 50%, 60%, 70%, 80%, 85%, 90%, 92%, at 93%, 94%, 95%, 96%, 97%, 98% or 99% of sequence identity on amino acid level or the DNA binding domain fragment of scTet, scArcR, TraR, LacR, LuxR, Gal4 and homologs of any one these having at least 50%, 60%, 70%, 80%, 85%, 90%, 92%, at 93%, 94%, 95%, 96%, 97%, 98% or 99% of sequence identity on amino acid level.

In one embodiment the heterologous DNA-binding domain is selected from the group consisting of: scTet, scArcR, TraR, LacR, LuxR and homologs of any one these having at least 50%, 60%, 70%, 80%, 85%, 90%, 92%, at 93%, 94%, 95%, 96%, 97%, 98% or 99% of sequence identity on amino acid level or the DNA binding domain fragment of scTet, scArcR, TraR, LacR, LuxR, Gal4 and homologs of any one these having at least 50%, 60%, 70%, 80%, 85%, 90%, 92%, at 93%, 94%, 95%, 96%, 97%, 98% or 99% of sequence identity on amino acid level.

In another embodiment the heterologous DNA-binding domain is scTet or scArcR and homologs of any one these having at least 50%, 60%, 70%, 80%, 85%, 90%, 92%, at 93%, 94%, 95%, 96%, 97%, 98% or 99% of sequence identity on amino acid level, or the DNA binding domain fragment of scTet or scArcR and homologs of any one these having at least 50%, 60%, 70%, 80%, 85%, 90%, 92%, at 93%, 94%, 95%, 96%, 97%, 98% or 99% of sequence identity on amino acid level.

In another embodiment the heterologous DNA-binding domain is scTet and homologs of scTet having at least 50%, 60%, 70%, 80%, 85%, 90%, 92%, at 93%, 94%, 95%, 96%, 97%, 98% or 99% of sequence identity on amino acid level, or the HTH domain of scTet and homologs of scTet having at least 50%, 60%, 70%, 80%, 85%, 90%, 92%, at 93%, 94%, 95%, 96%, 97%, 98% or 99% of sequence identity on amino acid level.

In another embodiment the heterologous DNA-binding domain is MarA and homologs of MarA having at least 50%, 60%, 70%, 80%, 85%, 90%, 92%, at 93%, 94%, 95%, 96%, 97%, 98% or 99% of sequence identity on amino acid level, or the HTH domain of MarA and homologs thereof having at least 50%, 60%, 70%, 80%, 85%, 90%, 92%, at 93%, 94%, 95%, 96%, 97%, 98% or 99% of sequence identity on amino acid level.

In another preferred embodiment, the heterologous DNA-dinging domain is a TAL effector protein or the DNA binding portion of a TAL effector. One may use native TAL effectors. Alternatively, TAL effectors can be designed to bind to certain recognition sequences (Moscou & Bogdanove, 2009, Science DOI: 10.1126/science. 1178817; Boch et al. 2009, Science DOI: 10.1126/science.1178811) and WO2010/079430 and EP2206723.

WO2010/079430 and EP2206723 are included herein by reference.

Examples for TAL effector proteins are AvBs3 (SEQ ID NO: 160), Hax2 (SEQ ID NO:161), Hax3 (SEQ ID NO: 162) and Hax4 (SEQ ID NO: 163).

The respective DNA binding site or the recognition sequence of

SEQ ID NO: 164)
AvBs3 is described by
5′-TCTNTAAACCTNNCCCTCT-3′, of
(SEQ ID NO: 165)
Hax2 is described by
5′-TGTTATTCTCACACTCTCCTTAT-3′, of,
(SEQ ID NO: 166)
Hax3 is described by
5′-TACACCCNNNCAT-3′
and
(SEQ ID NO: 167)
of Hax4 is described by
5′-TACCTNNACTANATAT-3′

Accordingly, in another embodiment, at least one heterologous DNA binding domain of the chimeric endonuclease is a TAL effector protein having an amino acid sequence identity of at least 80%, 81%, 82%, 83%, 84%, 85%, 86%, 87%, 88%, 89%, 90%, 91%, 92%, 93%, 94%, 95%, 96%, 97%, 98%, 99% to an amino acid sequence described by SEQ ID NO: 160, 161, 162 or 164, or a fragment of the DNA binding domain of a TAL effector protein having an amino acid sequence identity of at least 80%, 81%, 82%, 83%, 84%, 85%, 86%, 87%, 88%, 89%, 90%, 91%, 92%, 93%, 94%, 95%, 96%, 97%, 98%, 99% to an amino acid sequence described by SEQ ID NO: 160, 161, 162 or 164, comprising at least 1, 2, 3, 4, 5, 6, 7, 8, 9, 10, 11, 12, 13, 14, 15, 16, 17, 18, 19 or 20 repeat units derived from a transcription activator-like (TAL) effector, or a transcription activator-like (TAL) effector.

In another embodiment, at least one heterologous DNA dinding domain of the chimeric endonuclease is at least one repeat unit derived from a transcription activator-like (TAL) effector, or a transcription activator-like (TAL) effector.

The term “repeat unit” is used to describe the modular portion of a repeat domain from a TAL effector, or an artificial version thereof, that contains one or two amino acids in positions 12 and 13 of the amino acid sequence of a repeat unit that determine recognition of a base pair in a target DNA sequence that such amino acids confer recognition of, as follows: HD for recognition of C/G; NI for recognition of NT; NG for recognition of T/A; NS for recognition of C/G or NT or T/A or G/C; NN for recognition of G/C or NT; IG for recognition of T/A; N for recognition of C/G; HG for recognition of C/G or T/A; H for recognition of T/A; and NK for recognition of G/C.

(the amino acids H, D, I, G, S, K are described in one-letter code, whereby A, T, C, G refer to the DNA base pairs recognized by the amino acids)

The number of repeat units to be used in a repeat domain can be ascertained by one skilled in the art by routine experimentation. Generally, at least 1.5 repeat units are considered as a minimum, although typically at least about 8 repeat units will be used. The repeat units do not have to be complete repeat units, as repeat units of half the size can be used. A heterologous DNA binding domain of the invention can comprise, for example, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 46, 46.5, 47, 47.5, 48, 48.5, 49, 49.5, 50, 50.5 or more repeat units.

A typical consensus sequence of a repeat with 34 amino acids (in one-letter code) is shown below:

(SEQ ID NO: 128)
LTPEQVVAIASNGGGKQALETVQRLLPVLCQAHG

A further consensus sequence for a repeat unit with 35 amino acids (in one-letter code) is as follows:

(SEQ ID NO: 129)
LTPEQVVAIASNGGGKQALETVQRLLPVLCQAPHD

The repeat units which can be used in one embodiment of the invention have an identity with the consensus sequences described above of at least 35%, 40%, 50%, 60%, 70%, 75%, 80%, 85%, 90% or 95%.

In one embodiment of the invention, the heterologous DNA binding domain is a transcription activator-like (TAL) effector of the group of transcription activator-like (TAL) effectors described by: AvrBs3, AvrBs3˜repl6, AvrBs3-repl09, AvrHahI, AvrXa27, PthXo1, PthXo6, PthXo7, or the members of the Hax sub-family Hax2, Hax3, Hax4 and BrgII, or homologs of these having at least 50%, 60%, 70%, 80%, 85%, 90%, 92%, at 93%, 94%, 95%, 96%, 97%, 98% or 99% of sequence identity on amino acid level.

In one embodiment of the invention, the heterologous DNA binding domain is not a TAL-Effector protein or a TAL-Effector repeat unit.

### Preparation of Chimeric Endonucleases:

Endonucleases and the heterologous DNA binding domains can be combined in many alternative ways.

For example, it is possible, to combine more than one endonuclease with one or more heterologous DNA binding domain or to combine more than one heterologous DNA binding domain with one endonuclease. It is also possible to combine more than one endonuclease with more than one heterologous DNA binding domain.

The heterologous DNA-binding domain or the heterologous DNA-binding-domains can be fused at the N-terminal or at the C-terminal end of the endonuclease. It is also possible, to fuse one or more heterologous DNA binding domains at the N-terminal end and one or more heterologous DNA binding domains at the C-terminal end of the endonuclease. It is also possible to make alternating combinations of endonucleases and heterologous DNA binding domains.

In case the chimeric endonuclease comprises more than one endonuclease or more than one heterologous DNA binding domain or more than one endonuclease and more than one heterologous DNA binding domain, it is possible to use several copies of the same heterologous DNA binding domain or endonuclease or to use different heterologous DNA binding domains or endonucleases.

It is also possible to apply the methods and features described for optimized nucleases above, to the full sequence of chimeric endonucleases, e.g. by adding a nuclear localization signal to a chimeric endonuclease or by reducing the number of:
a) PEST-Sequences,
b) KEN-boxes
c) A-boxes,
d) D-boxes, or
e) comprise an optimized N-terminal end for stability according to the N-end rule,
f) comprise a glycin as the second N-terminal amino acid, or
g) any combination of a), b), c) d), e) and f). of the entire amino acid sequence of the chimeric endonuclease.

Chimeric endonucleases having a nuclear localization signal are for example described by the amino acid sequence described by SEQ ID NO: 11, or the polynucleotide sequence described by SEQ ID NO: 24, 25 or 26.

In one embodiment the chimeric endonucleases are combinations of:

I-SceI and scTet, or I-SceI and scArc, or I-CreI and scTet, or I-CreI and scArcR or I-MsoI and scTet, or I-MsoI and scArcR, wherein scTet, or scArcR are fused N- or C-terminal to I-SceI, I-CreI or I-MsoI and wherein I-SceI, I-CreI, I-MsoI, scTet, scArcR, include their homologs having at least 50%, 49%, 51%, 58%, 60%, 70%, 80%, 85%, 90%, 92%, at 93%, 94%, 95%, 96%, 97%, 98% or 99% of sequence identity on amino acid level.

In another embodiment the chimeric endonucleases have the following structure:
N-terminus-I-SceI-scTet-C-terminus, or
N-terminus-I-SceI-scArcR-C-terminus, or
N-terminus-I-CreI-scTet-C-terminus, or
N-terminus-I-CreI-scArcR-C-terminus, or
N-terminus-I-MsoI-scTet-C-terminus, or
N-terminus-I-MsoI-scArcR-C-terminus,
N-terminus-scTet-I-SceI-C-terminus, or
N-terminus-scArcR-I-SceI-C-terminus, or
N-terminus-scTet-I-CreI-C-terminus, or
N-terminus-scArcR-I-CreI-C-terminus, or
N-terminus-scTet-I-MsoI-C-terminus, or
N-terminus-scArcR-I-MsoI-C-terminus,

The chimeric endonuclease is preferably expressed as a fusion protein with a nuclear localization sequence (NLS). This NLS sequence enables facilitated transport into the nucleus and increases the efficacy of the recombination system. A variety of NLS sequences are known to the skilled worker and described, inter alia, by Jicks G R and Raikhel N V (1995) Annu. Rev. Cell Biol. 11:155-188. Preferred for plant organisms is, for example, the NLS sequence of the SV40 large antigen. Examples are provided in WO 03/060133 included herein by reference. The NLS may be heterologous to the endonuclease and/or the DNA binding domain or may be naturally comprised within the endonuclease and/or DNA binding domain.

In a preferred embodiment, the sequences encoding the chimeric endonucleases are modified by insertion of an intron sequence. This prevents expression of a functional enzyme in procaryotic host organisms and thereby facilitates cloning and transformations procedures (e.g., based on E. coli or Agrobacterium). In eukaryotic organisms, for example plant organisms, expression of a functional enzyme is realized, since plants are able to recognize and “splice” out introns. Preferably, introns are inserted in the homing endonucleases mentioned as preferred above (e.g., into I-SceI or I-CreI).

In another preferred embodiment, the amino acid sequences of the endonuclease or the chimeric endonuclease can be modified by adding a Sec IV secretion signal to the N-, or C-Terminus of the endonuclease or chimeric endonuclease.

In a preferred embodiment the SecIV secretion signal is a SecIV secretion signal comprised in Vir proteins of Agrobacterium. Examples of such Sec IV secretion signals as well as methods how to apply these are disclosed in WO 01/89283, in Vergunst et al, Positive charge is an important feature of the C-terminal transport signal of the VirB/D4-translocated proteins of Agrobacterium, PNAS 2005, 102, 03, pages 832 to 837 included herein by reference. A Sec IV secretion signal might also be added, by adding fragments of a Vir protein or even a complete Vir protein, for example a complete VirE2 protein to a endonuclease or chimeric endonuclease, in a similar way as described in the description of WO01/38504 included herein by reference, which describes a RecA/VirE2 fusion protein.

In another preferred embodiment the amino acid sequences of the endonuclease or the chimeric endonuclease can be modified by adding a Sec III secretion signal to the N-, or C-Terminus of the endonuclease or chimeric endonuclease. Suitable SecIII secretion signals are for example disclosed in WO 00/02996, included herein by reference.

In case a Sec III secretion signal is added, it can be of advantage, to express this endonuclease or chimeric endonuclease in a cell, which does also comprise a recombinant construct encoding parts of, or a complete functional type III secretion system, in order to overexpress or complement parts or the complete functional type III secretion system in such cell.

Recombinant constructs encoding parts or a complete functional type III secretion system are for example disclosed in WO 00/02996 and WO05/085417 included herein by reference.

If a SecIV secretion signal is added to the chimeric endonuclease and the chimeric endonuclease is intended to be expressed for example in Agrobacterium rhizogenes or in Agrobacterium tumefaciens, it is of advantage to adapt the DNA sequence coding for the chimeric endonuclease to the codon usage of the expressing organism. Preferably the endonuclease or chimeric nuclease does not have or has only few DNA recognition sequences in the genome of the expressing organism. It is of even greater advantage, if the selected chimeric endonuclease does not have a DNA recognition sequence or less preferred DNA recognition sequence in the Agrobacterium genome. In case the nuclease or the chimeric endonuclease is intended to be expressed in a prokaryotic organism the nuclease or chimeric nuclease encoding sequence must not have an intron.

In one embodiment the endonuclease and the heterologous DNA binding domain are connected via a linker polypeptide.

Preferably the linker polypeptide consists of 1 to 30 amino acids, more preferred 1 to 20 and even more preferred 1 to 10 amino acids.

For example, the linker polypeptide can be composed of a plurality of residues selected from the group consisting of glycine, serine, threonine, cysteine, asparagine, glutamine, and proline. Preferably the linker polypeptide is designed to lack secondary structures under physiological conditions and is preferably hydrophilic. Charged or non polar residues may be included, but they may interact to form secondary structures or may reduce solubility and are therefore less preferred.

In some embodiments the linker polypeptide consists essentially of a plurality of residues selected from glycine and serine. Examples of such linkers have the amino acid sequence (in one letter code): GS, or GGS, or GSGS, or GSGSGS, or GGSGG, or GGSGGSGG, or GSGSGGSG.

In case the linker consists of at least 3 amino acids, it is preferred that the amino acid sequence of the linker polypeptide comprises at least one third Glycines or Alanines or Glycines and Alanines.

In one preferred embodiment, the linker sequence has the amino acid sequence GSGS or GSGSGS.

Preferably the polypeptide linker is rationally designed using bioinformatic tools, capable of modeling both the DNA-binding site and the respective edonuclease, as well as the recognition site and the heterologous DNA-binding domain. Suitable bioinformatic tools are for example described in Desjarlais & Berg, (1994), PNAS, 90, 2256 to 2260 and in Desjarlais & Berg (1994), PNAS, 91, 11099 to 11103.

### DNA Recognition Sequences of Chimeric Endonucleases (Chimeric Recognition Sequences):

The chimeric endonucleases bind to DNA sequences being combinations of the DNA recognition sequence of the endonuclease and the recognition sequence of the heterologous DNA binding domain. In case the chimeric endonuclease comprises more than one endonuclease or more than one heterologous DNA binding domain the DNA the chimeric endonuclease will bind to DNA sequences being a combination of the DNA recognition sequence of the endonucleases used and the operator sequences of the heterologous DNA binding domains used. It is clear, that the sequence of the DNA, which is bound by the chimeric endonuclease will reflect the order, in which the endonuclease and the heterologous DNA binding domains are combined.

Endonucleases known in the art cut a huge variety of different polynucleotide sequences. The terms DNA recognition sequence and DNA recognition site are used synonymously and refer to a polynucleotide of a particular sequence which can be bound and cut by a given endonuclease. A polynucleotide of a given sequence may therefore be a DNA recognition sequence or DNA recognition site for one endonuclease, but may or may not be a DNA recognition sequence or DNA recognition site for another endonuclease.

Examples of polynucleotide sequences which can be bound and cut by endonucleases, i.e. which represent a DNA recognition sequence or DNA recognition site for this endonuclease, are described in Table 8: the letter N represents any nucleotide, and can be replaced by A, T, G or C).

TABLE 8

| Endonu- | Organism | |
|---|---|---|
| clease | of origin | DNA recognition sequence |
| I-CreI | Chlamydomonas | 5′-CAAAACGTCGTGAGACAGTTTC-3′ |
| | reinhardtii | (SEQ ID NO: 138) |
| I-CeuI | Chlamydomonas | 5′-ATAACGGTCCTAAGGTAGCGAA-3′ |
| | eugametos | (SEQ ID NO: 139) |
| I-DmoI | Desulfuro- | 5′-ATGCCTTGCCGGGTAAGTTCCGGCGCGCAT-3′ |
| | coccus mobilis | (SEQ ID NO: 140) |
| I-MsoI | Monomastix spec. | 5′-CAGAACGTCGTGAGACAGTTCC-3′ |
| | | (SEQ ID NO: 153) |
| PI-PsiI | S. cerrevisia | 5′-ATCTATGTCGGGTGCGGAGAAAGAGGTAAT-3′ |
| | | (SEQ ID NO: 154) |
| I-AniI | Aspergillus nidulans | 5′-GCGCGCTGAGGAGGTTTCTCTGTAAAGCGCA-3′ |
| | | (SEQ ID NO: 142) |

Endonucleases do not have stringently-defined DNA recognition sequences, so that single base changes do not abolish cleavage but may reduce its efficiency to variable extents. A DNA recognition sequence listed herein for a given endonuclease represents only one site that is known to be recognized and cleaved.

Examples for deviations of a DNA recognition site are for example disclosed in Chevelier et al. (2003), J. Mol. Biol. 329, 253 to 269, in Marcaida et al. (2008), PNAS, 105 (44), 16888 to 16893 and in the Supporting Information to Marcaida et al. 10.1073/pnas.0804795105, in Doyon et al. (2006), J. AM. CHEM. SOC. 128, 2477 to 2484, in Argast et al, (1998), J. Mol. Biol. 280, 345 to 353, in Spiegel et al. (2006), Structure, 14, 869 to 880, in Posey et al. (2004), Nucl. Acids Res. 32 (13), 3947 to 3956, or in Chen et al. (2009), Protein Engineering, Design & Selection, 22 (4), 249 to 256.

It is therefore possible to identify a naturally occurring endonuclease having a predetermined polynucleotide sequence as a DNA recognition sequence.

Methods to identify naturally occurring endonucleases, their genes and their DNA recognition sequences are disclosed for example in WO 2009/101625.

The cleavage specificity or respectively its degeneration of its DNA recognition sequence can be tested by testing its activity on different substrates. Suitable in vivo techniques are for example disclosed in WO09074873.

Alternatively, in vitro tests can be used, for example by employing labeled polynucleotides spotted on arrays, wherein different spots comprise essentially only polynucleotides of a particular sequence, which differs from the polynucleotides of different spots and which may or may not be DNA recognition sequences of the endonuclease to be tested for its activity. A similar technique is disclosed for example in US 2009/0197775.

However, it is possible to mutate the amino acid sequence of a given endonuclease, preferably a LAGLIDADG endonuclease, to bind and cut new polynucleotides, i.e. creating an engineered endonuclease having a changed DNA recognition site.

Numerous examples DNA recognition sites of engineered endonucleases are known in the art and are disclosed for example in WO 2005/105989, WO 2007/034262, WO 2007/047859, WO 2007/093918, WO 2008/093249, WO 2008/102198, WO 2008/152524, WO 2009/001159, WO 2009/059195, WO 2009/076292, WO 2009/114321, or WO 2009/134714 WO 10/001,189, and WO 10/009,147.

Therefore it is also possible to create an engineered endonuclease which will have a DNA recognition sequence identical to a particular predetermined polynucleotide sequence.

Preferably the DNA recognition sequence of the endonuclease and the operator sequence are separated by 1, 2, 3, 4, 5, 6, 7, 8, 9, 10 or more base pairs. Preferably they are separated by 1 to 10, 1 to 8, 1 to 6, 1 to 4, 1 to 3, or 2 base pairs.

The amount of base pairs used to separate the DNA recognition sequence of the nuclease and the recognition sequence of the heterologous DNA binding domain depends on the distance of the DNA binding regions of the nuclease and the DNA binding region of the heterologous DNA binding domain in the chimeric endonuclease. A larger distance between the DNA binding regions of the nuclease and the DNA binding region of the heterologous DNA binding domain will be reflected by a higher amount of base pairs separating the DNA recognition sequence of the nuclease and the recognition sequence of the heterologous DNA binding domain. The optimal amount of separating base pairs can be determined by using computer models or by testing the binding and cutting efficiency of a given chimeric endonuclease on several polynucleotides comprising a varying amount of base pairs between the DNA recognition sequence of the nuclease and the recognition sequence of the heterologous DNA binding domain.

Accordingly, in one embodiment of the invention, the chimeric recognition site comprises a DNA recognition sequence of a LAGLIDADG endonuclease, even more preferred a DNA recognition sequence of a LAGLIDADG endonuclease having an amino acid sequence as described by at least one of SEQ ID NOs: 1, 2, 3, 5, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 142 or 159, preferably having an amino acid sequence as described by SEQ ID NO: 1, 2, 3, 5 or 159.

In a further embodiment of the invention, the chimeric recognition site comprises a DNA recognition sequence of I-SceI, I-CreI, I-DmoI, I-MsoI, I-CeuI, I-ChuI, Pi-SceI or I-AniI or a homolog of these having at least 56%, 57%, 58%, 59%, 60%, 61%, 62%, 63%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% to I-SceI, I-CreI, I-DmoI, I-MsoI, I-CeuI, I-ChuI, Pi-SceI or I-AniI, and a recognition sequence of a heterologous DNA binding domain having at least 50% sequence amino acid sequence identity to scTet, scArc, LacR, MerR or MarA or to a DNA binding domain fragment of scTet, scArc, LacR, MerR or MarA.

In a further embodiment of the invention, the chimeric recognition site comprises a two DNA recognition sequences of I-SceI, I-CreI, I-DmoI, I-MsoI, I-CeuI, I-ChuI, Pi-SceI or I-AniI or a homolog of these having at least 56%, 57%, 58%, 59%, 60%, 61%, 62%, 63%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% to I-SceI, I-CreI, I-DmoI, I-MsoI, I-CeuI, I-ChuI, Pi-SceI or I-AniI.

Such chimeric recognition sites can be used with chimeric endonucleases comprising an active endonuclease and an inactive endonuclease as heterologous DNA binding domain. One example for such types of combinations are a chimeric recognition site comprising two DNA recognition sequences of I-SceI, which can be used in combination with a chimeric endonuclease comprising an active version of I-SceI and an inactive version of I-SceI as heterologous DNA binding domain.

In a further embodiment of the invention, the chimeric recognition site comprises a two DNA recognition sequences of I-SceI, I-CreI, I-DmoI, I-MsoI, I-CeuI, I-ChuI, Pi-SceI or I-AniI or a homolog of these having at least 56%, 57%, 58%, 59%, 60%, 61%, 62%, 63%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% to I-SceI, I-CreI, I-DmoI, I-MsoI, I-CeuI, I-ChuI, Pi-SceI or I-AniI and a DNA binding site of a TAL-effector protein, preferably comprising a polynucleotide sequence as described by SEQ ID NO: 164, 165, 166 or 167.

In another embodiment of the invention, the chimeric recognition site comprises a two DNA recognition sequences of I-SceI, preferably described by SEQ ID NO: 13 and a DNA binding site of a TAL-effector protein, preferably comprising a polynucleotide sequence as described by SEQ ID NO: 164, 165, 166 or 167.

Examples for DNA recognition sequences of chimeric endonucleases (chimeric recognition site or target site of the respective chimeric endonuclease) are:

A chimeric endonuclease having the structure: I-SceI-scTet, preferably having an amino acid sequence described by SEQ ID NO: 8 or 9

I-SceI scTet target site 1
(SEQ ID NO: 14)
ctatcaatgatagcgctagggataacagggtaat
I-SceI scTet target site 2
(SEQ ID NO: 15)
ctatcaatgatagacgctagggataacagggtaat
I-SceI scTet target site 3
(SEQ ID NO: 16)
ctatcaatgatagtacgctagggataacagggtaat

A chimeric endonuclease having the structure: I-SceI-scArcR, preferably having an amino acid sequence described by SEQ ID NO: 10 or 11

I-SceI scArc target site 1
(SEQ ID NO: 17)
tagggataacagggtaatactagtagagtgc
I-SceI scArc target site 2
(SEQ ID NO: 18)
tagggataacagggtaatacttagtagagtgc
I-SceI scArc target site 3
(SEQ ID NO: 19)
tagggataacagggtaatactatagtagagtgc
I-SceI scArc target site 4
(SEQ ID NO: 20)
tagggataacagggtaatactagtagtagagtgc

### Polynucleotides:

The invention does also comprise isolated polynucleotides coding for the chimeric endonucleases described above.

Examples of such isolated polynucleotides are isolated polynucleotides coding for amino acid sequences described by SEQ ID NO: 23, 24, 25 and 26 or amino acid sequences having at least 70%, 80%, 90% 91%, 92%, 93%, 94%, 95%, 96%, 97%, 98% or 99% amino acid sequence similarity, preferably having at least 70%, 80%, 90% 91%, 92%, 93%, 94%, 95%, 96%, 97%, 98% or 99% amino acid sequence identity to any one of the amino acid sequences described by SEQ ID NO: 23, 24, 25 and 26.

Preferably the isolated polynucleotide has a optimized codon usage for expression in a particular host organism, or has a low content of RNA instability motifs, or has a low content of codon repeats, or has a low contend of cryptic splice sites, or has a low content of alternative start codons, or has a low content of restriction sites, or has a low content of RNA secondary structures or has any combination of these features.

The codon usage of the isolated polypeptide may be optimized e.g. for the expression in plants, preferably in a plant selected from the group comprising: rice, corn, wheat, rape seed, sugar cane, sunflower, sugar beet, tobacco.

Preferably the isolated polynucleotide is combined with a promoter sequence and a terminator sequence suitable to form a functional expression cassette for expression of the chimeric endonuclease in a particular host organism.

Suitable promoters are for example constitutive, heat- or pathogen-inducible, or seed, pollen, flower or fruit specific promoters.

The person skilled in the art knows numerous promoters having those features.

For example several constitutive promoters in plants are known. Most of them are derived from viral or bacterial sources such as the nopaline synthase (nos) promoter (Shaw et al. (1984) Nucleic Acids Res. 12 (20): 7831-7846), the mannopine synthase (mas) promoter (Co-mai et al. (1990) Plant Mol Biol 15(3):373-381), or the octopine synthase (ocs) pro-moter (Leisner and Gelvin (1988) Proc Natl Acad Sci USA 85 (5):2553-2557) from Agrobacterium tumefaciens or the CaMV35S promote from the Cauliflower Mosaic Virus (U.S. Pat. No. 5,352,605). The latter was most frequently used in constitutive expression of transgenes in plants (Odell et al. (1985) Nature 313:810-812; Battraw and Hall (1990) Plant Mol Biol 15:527-538; Benfey et al. (1990) EMBO J. 9(69):1677-1684; U.S. Pat. No. 5,612,472). However, the CaMV 35S promoter demonstrates variability not only in different plant species but also in different plant tissues (Atanassova et al. (1998) Plant Mol Biol 37:275-85; Battraw and Hall (1990) Plant Mol Biol 15:527-538; Holtorf et al. (1995) Plant Mol Biol 29:637-646; Jefferson et al. (1987) EMBO J. 6:3901-3907). An additional disadvantage is an interference of the transcription regulating activity of the 35S promoter with wild-type CaMV virus (Al-Kaff et al. (2000) Nature Biotechnology 18:995-99). Another viral promoter for constitutive expression is the Sugarcane bacilliform badnavirus (ScBV) promoter (Schenk et al. (1999) Plant Mol Biol 39 (6):1221-1230).

Several plant constitutive promoters are described such as the ubiquitin promoter from Arabidopsis thaliana (Callis et al. (1990) J Biol Chem 265:12486-12493; Holtorf S et al. (1995) Plant Mol Biol 29:637-747), which—however—is reported to be unable to regulate expression of selection markers (WO03102198), or two maize ubiquitin promoter (Ubi-1 and Ubi-2; U.S. Pat. No. 5,510,474; U.S. Pat. No. 6,020,190; U.S. Pat. No. 6,054,574), which beside a constitutive expression profile demonstrate a heat-shock induction (Christensen et al. (1992) Plant. Mol. Biol. 18(4):675-689). A comparison of specificity and expression level of the CaMV 35S, the barley thionine promoter, and the Arabidopsis ubiquitin promoter based on stably transformed Arabidopsis plants demonstrates a high expression rate for the CaMV 35S promoter, while the thionine promoter was inactive in most lines and the ubi1 promoter from Arabisopsis resulted only in moderate expression activity (Holtorf et al. (1995) Plant Mol Biol 29 (4):637-6469).

### Chimeric Recognition Sequences:

The invention does also comprise isolated polynucleotides comprising a chimeric recognition sequence, having a length of about 15 to about 300, or of about 20 to about 200 or of about 25 to about 100 nucleotides, comprising a DNA recognition sequence of an endonuclease and a recognition sequence of a heterologous DNA binding domain (also called binding site or operator)

Preferably isolated polynucleotides comprise a DNA recognition sequence of a homing endonuclease, preferably of a LAGLIDADG endonuclease.

In one embodiment the isolated polynucleotide comprises a DNA recognition sequence of 1-SceI.

Preferably the recognition sequence of a heterologous DNA binding domain comprised in the isolated polynucleotide is a recognition sequence of a transcription factor.

More preferably the recognition sequence is the recognition sequence of the transcription factors scTet or scArc.

In one embodiment the isolated polynucleotide comprises a DNA recognition sequence of I-SceI and a linker sequence of 0 to 10 polynucleotides and a recognition sequence of scTet or scArc.

Preferred chimeric recognition sequences comprise a combination of a DNA recognition sequence of I-SceI, I-CreI, I-DmoI, or I-Ceu, I-MsoI, Pi-SceI or I-AniI in combination with a recognition site of scTet, TetR, scArcR, TraR, WRKY, LacR, MarA or MerR, wherein the DNA recognition sequence of I-SceI, I-CreI, I-DmoI, I-MsoI, or I-Ceu may be fused in a distance of 1, 2, 3, 4, 5, 6, 7, 8, 9, 10, 11 or 12 nucleotides up or downstream of a recognition site of scTet, TetR, scArcR, TraR, WRKY, LacR, MarA or MerR.

Preferred chimeric recognition sequences comprise a combination of a DNA recognition sequence of I-SceI, I-CreI, I-DmoI, or I-MsoI in combination with a recognition site of scTet, TetR, scArcR, TraR, MarA or MerR, wherein the DNA recognition sequence of I-SceI, I-CreI, I-DmoI, or I-Ceu may be fused in a distance of 1, 2, 3, 4, 5, 6, 7, 8, 9, 10, 11 or 12 nucleotides up or downstream of a recognition site of scTet, TetR, scArcR, TraR, MarA or MerR.

Preferred chimeric recognition sequences comprise a combination of a DNA recognition sequence of I-SceI, I-CreI, I-DmoI or I-MsoI in combination with a recognition site of scTet, TetR, scArcR, TraR, MarA or MerR, wherein the DNA recognition sequence of I-SceI, I-CreI, I-DmoI, or I-Ceu may be fused in a distance of 1, 2, 3, 4, 5, 6, 7, 8, 9, 10, 11 or 12 nucleotides up or downstream of a recognition site of scTet, TetR, scArcR, TraR, MarA or MerR.

In one embodiment, the chimeric recognition sequence comprise a combination a DNA recognition sequence of I-SceI in combination with a recognition site of scTet, TetR, scArcR, TraR, MarA or MerR, wherein the DNA recognition sequence of I-SceI may be fused in a distance of 1, 2, 3, 4, 5, 6, 7, 8, 9, 10, 11 or 12 nucleotides up or downstream of a recognition site of scTet, TetR, scArcR, TraR, MarA or MerR.

In one embodiment, the chimeric recognition sequence comprise a combination a DNA recognition sequence of I-SceI in combination with a recognition site of MarA wherein the DNA recognition sequence of I-SceI may be fused in a distance of 1, 2, 3, 4, 5, 6, 7, 8, 9, 10, 11 or 12 nucleotides up or downstream of a recognition site of MarA. Preferably, the DNA recognition sequence of I-SceI is fused upstream of a recognition site of MarA.

In one embodiment the isolated polynucleotide comprise a sequence of a chimeric recognition site selected from the group comprising: SEQ ID NO: 30, 31, 32, 34, 35, 36 or 37.

The isolated polynucleotides may comprise a combination of a chimeric recognition site and a polynucleotide sequence coding for a chimeric nuclease.

In a preferred embodiment of the invention, a chimeric endonuclease having an amino acid sequence as described by SEQ ID NO: 8 or 9, is used in combination with a chimeric recognition sequence having a polynucleotide sequence selected from the group of sequences described by: SEQ ID NO: 14, 15 or 16.

In a preferred embodiment of the invention, a chimeric endonuclease having an amino acid sequence as described by SEQ ID NO: 10 or 11, is used in combination with a chimeric recognition sequence having a polynucleotide sequence selected from the group of sequences described by: SEQ ID NO: 17, 18, 19 or 20.

### Vectors:

The polynucleotides described above may be comprised in a DNA vector suitable for transformation, transfection, cloning or overexpression.

In one example, the polynucleotides described above are comprised in a vector for transformation of non-human organisms or cells, preferably the non-human organisms are plants or plant cells.

The vectors of the invention usually comprise further functional elements, which may include but shall not be limited to:

i) Origins of replication which ensure replication of the expression cassettes or vectors according to the invention in, for example, E. coli. Examples which may be mentioned are ORI (origin of DNA replication), the pBR322 on or the P15A on (Sam-brook et al.: Molecular Cloning. A Laboratory Manual, 2nd ed. Cold Spring Harbor Laboratory Press, Cold Spring Harbor, N.Y., 1989)
ii) Multiple cloning sites (MCS) to enable and facilitate the insertion of one or more nucleic acid sequences.
iii) Sequences which make possible homologous recombination or insertion into the genome of a host organism.
iv) Elements, for example border sequences, which make possible the Agrobacterium-mediated transfer in plant cells for the transfer and integration into the plant genome, such as, for example, the right or left border of the T-DNA or the vir region.

### The Marker Sequence

The term “marker sequence” is to be understood in the broad sense to include all nucleotide sequences (and/or polypeptide sequences translated therefrom) which facilitate detection, identification, or selection of transformed cells, tissues or organism (e.g., plants). The terms “sequence allowing selection of a transformed plant material”, “selection marker” or “selection marker gene” or “selection marker protein” or “marker” have essentially the same meaning.

Markers may include (but are not limited to) selectable marker and screenable marker. A selectable marker confers to the cell or organism a phenotype resulting in a growth or viability difference. The selectable marker may interact with a selection agent (such as a herbicide or anti-biotic or pro-drug) to bring about this phenotype. A screenable marker confers to the cell or organism a readily detectable phenotype, preferably a visibly detectable phenotype such a color or staining. The screenable marker may interact with a screening agent (such as a dye) to bring about this phenotype.

Selectable marker (or selectable marker sequences) comprise but are not limited to

a) negative selection marker, which confers resistance against one or more toxic (in case of plants phytotoxic) agents such as an antibiotica, herbicides or other biocides,
b) counter selection marker, which confer a sensitivity against certain chemical compounds (e.g., by converting a non-toxic compound into a toxic compound), and
c) positive selection marker, which confer a growth advantage (e.g., by expression of key elements of the cytokinin or hormone biosynthesis leading to the production of a plant hormone e.g., auxins, gibberllins, cytokinins, abscisic acid and ethylene; Ebi-numa H et al. (2000) Proc Natl Acad Sci USA 94:2117-2121).

When using negative selection markers, only cells or plants are selected which comprise said negative selection marker. When using counter selection marker, only cells or plants are selected which lack said counter-selection marker. Counter-selection marker may be employed to verify successful excision of a sequence (comprising said counter-selection marker) from a genome. Screenable marker sequences include but are not limited to reporter genes (e.g. luciferase, glucuronidase, chloramphenicol acetyl transferase (CAT, etc.). Preferred marker sequences include but shall not be limited to:

### i) Negative Selection Marker

As a rule, negative selection markers are useful for selecting cells which have success-fully undergone transformation. The negative selection marker, which has been introduced with the DNA construct of the invention, may confer resistance to a biocide or phytotoxic agent (for example a herbicide such as phosphinothricin, glyphosate or bromoxynil), a metabolism inhibitor such as 2-deoxyglucose-6-phosphate (WO 98/45456) or an antibiotic such as, for example, tetracyclin, ampicillin, kanamycin, G 418, neomycin, bleomycin or hygromycin to the cells which have successfully under-gone transformation. The negative selection marker permits the selection of the trans-formed cells from untransformed cells (McCormick et al. (1986) Plant Cell Reports 5:81-84). Negative selection marker in a vector of the invention may be employed to confer resistance in more than one organism. For example a vector of the invention may comprise a selection marker for amplification in bacteria (such as E. coli or Agrobacterium) and plants. Examples of selectable markers for E. coli include: genes specifying resistance to antibiotics, i.e., ampicillin, tetracycline, kanamycin, erythromycin, or genes conferring other types of selectable enzymatic activities such as galactosidase, or the lactose operon. Suitable selectable markers for use in mammalian cells include, for example, the dihydrofolate reductase gene (DHFR), the thymidine kinase gene (TK), or prokaryotic genes conferring drug resistance, gpt (xanthine-guanine phosphoribosyltransferase, which can be selected for with mycophenolic acid; neo (neomycin phosphotransferase), which can be selected for with G418, hygromycin, or puromycin; and DHFR (dihydrofolate reductase), which can be selected for with methotrexate (Mulligan & Berg (1981) Proc Natl Acad Sci USA 78:2072; Southern & Berg (1982) J Mol Appl Genet. 1: 327). Selection markers for plant cells often confer resistance to a biocide or an antibiotic, such as, for example, kanamycin, G 418, bleomycin, hygromycin, or chloramphenicol, or herbicide resistance, such as resistance to chlorsulfuron or Basta.

Especially preferred negative selection markers are those which confer resistance to herbicides. Examples of negative selection markers are
DNA sequences which encode phosphinothricin acetyltransferases (PAT), which acetylates the free amino group of the glutamine synthase inhibitor phosphinothricin (PPT) and thus brings about detoxification of PPT (de Block et al. (1987) EMBO J. 6:2513-2518) (also referred to as Bialophos-resistance gene bar; EP 242236),
5-enolpyruvylshikimate-3-phosphate synthase genes (EPSP synthase genes), which confer resistance to Glyphosate-(N-(phosphonomethyl)glycine),
the gox gene, which encodes the Glyphosate-degrading enzyme Glyphosate oxi-doreductase,
the deh gene (encoding a dehalogenase which inactivates Dalapon-),
acetolactate synthases which confer resistance to sulfonylurea and imidazolinone,
bxn genes which encode Bromoxynil-degrading nitrilase enzymes,
the kanamycin, or G418, resistance gene (NPTII). The NPTII gene encodes a neomycin phosphotransferase which reduces the inhibitory effect of kanamycin, neomycin, G418 and paromomycin owing to a phosphorylation reaction (Beck et al (1982) Gene 19: 327),
the DOGR1 gene. The DOGR1 gene has been isolated from the yeast Saccharomyces cerevisiae (EP 0 807 836). It encodes a 2-deoxyglucose-6-phosphate phos-phatase which confers resistance to 2-DOG (Randez-Gil et al. (1995) Yeast 11:1233-1240).
the hyg gene, which codes for the enzyme hygromycin phosphotransferase and confers resistance to the antibiotic hygromycin (Gritz and Davies (1983) Gene 25: 179);
especially preferred are negative selection markers that confer resistance against the toxic effects imposed by D-amino acids like e.g., D-alanine and D-serine (WO 03/060133; Erikson 2004). Especially preferred as negative selection marker in this contest are the daol gene (EC: 1.4. 3.3: GenBank Acc.-No.: U60066) from the yeast Rhodotorula gracilis (Rhodosporidium toruloides) and the E. coli gene dsdA (D-serine dehydratase (D-serine deaminase) (EC: 4.3.1.18; GenBank Acc.-No.: J01603).

### ii) Positive Selection Marker

Positive selection marker comprise but are not limited to growth stimulating selection marker genes like isopentenyltransferase from Agrobacterium tumefaciens (strain: PO22; Genbank Acc.-No.: AB025109) may—as a key enzyme of the cytokinin biosynthesis—facilitate regeneration of transformed plants (e.g., by selection on cytokinin-free medium). Corresponding selection methods are described (Ebinuma H et al. (2000) Proc Natl Acad Sci USA 94:2117-2121; Ebinuma H et al. (2000) Selection of Marker-free transgenic plants using the oncogenes (ipt, rol A, B, C) of Agrobacterium as selectable markers, In Molecular Biology of Woody Plants. Kluwer Academic Publishers). Additional positive selection markers, which confer a growth advantage to a transformed plant in comparison with a non-transformed one, are described e.g., in EP-A 0 601 092. Growth stimulation selection markers may include (but shall not be limited to) beta-Glucuronidase (in combination with e.g., a cytokinin glucuronide), mannose-6-phosphate isomerase (in combination with mannose), UDP-galactose-4-epimerase (in combination with e.g., galactose), wherein mannose-6-phosphate isomerase in combination with mannose is especially preferred.

iii) Counter Selection Markers

Counter-selection marker enable the selection of organisms with successfully deleted sequences (Koprek T et al. (1999) Plant J 19(6):719-726). TK thymidine kinase (TK) and diphtheria toxin A fragment (DT-A), codA gene encoding a cytosine deaminase (Gleve A P et al. (1999) Plant Mol Biol 40(2):223-35; Pereat R1 et al. (1993) Plant Mol Biol 23(4):793-799; Stougaard J (1993) Plant J 3:755-761), the cytochrome P450 gene (Koprek et al. (1999) Plant J 16:719-726), genes encoding a haloalkane dehalogenase (Naested H (1999) Plant J 18:571-576), the iaaH gene (Sundaresan V et al. (1995) Genes & Development 9:1797-1810), the tms2 gene (Fedoroff N V & Smith D L (1993) Plant J 3:273-289), and D-amino acid oxidases causing toxic effects by conversion of D-amino acids (WO 03/060133).

In a preferred embodiment the excision cassette includes at least one of said counter-selection markers to distinguish plant cells or plants with successfully excised sequences from plant which still contain these. In a more preferred embodiment the excision cassette of the invention comprises a dual-function marker i.e. a marker with can be employed as both a negative and a counter selection marker depending on the substrate employed in the selection scheme. An example for a dual-function marker is the daol gene (EC: 1.4. 3.3: GenBank Acc.-No.: U60066) from the yeast Rhodotorula gracilis, which can be employed as negative selection marker with D.-amino acids such as D-alanine and D-serine, and as counter-selection marker with D-amino acids such as D-isoleucine and D-valine (see European Patent Appl. No.: 04006358.8)

### iv) Screenable Marker (Reporter Genes)

Screenable marker (such as reporter genes) encode readily quantifiable or detectable proteins and which, via intrinsic color or enzyme activity, ensure the assessment of the transformation efficacy or of the location or timing of expression. Especially preferred are genes encoding reporter proteins (see also Schenborn E, Groskreutz D. (1999) Mol Biotechnol 13(1):29-44) such as
“green fluorescence protein” (GFP) (Chuff W L et al. (1996) Curr Biol 6:325-330; Lef-fel S M et al. (1997) Biotechniques 23(5):912-8; Sheen et al. (1995) Plant J 8(5):777-784; Haseloff et al. (1997) Proc Natl Acad Sci USA 94(6):2122-2127; Reichel et al. (1996) Proc Natl Acad Sci USA 93(12):5888-5893; Tian et al. (1997) Plant Cell Rep 16:267-271; WO 97/41228).
Chloramphenicol transferase,
luciferase (Millar et al. (1992) Plant Mol Biol Rep 10:324-414; Ow et al. (1986) Science 234:856-859) permits selection by detection of bioluminescence,
beta-galactosidase, encodes an enzyme for which a variety of chromogenic substrates are available,
beta-glucuronidase (GUS) (Jefferson et al. (1987) EMBO J. 6:3901-3907) or the uidA gene, which encodes an enzyme for a variety of chromogenic substrates,
R locus gene product: protein which regulates the production of anthocyanin pigments (red coloration) in plant tissue and thus makes possible the direct analysis of the promoter activity without the addition of additional adjuvants or chromogenic substrates (Dellaporta et al. (1988) In: Chromosome Structure and Function: Impact of New Concepts, 18th Stadler Genetics Symposium, 11:263-282),
beta-lactamase (Sutcliffe (1978) Proc Natl Acad Sci USA 75:3737-3741), enzyme for a variety of chromogenic substrates (for example PADAC, a chromogenic cepha-losporin),
xylE gene product (Zukowsky et al. (1983) Proc Natl Acad Sci USA 80:1101-1105), catechol dioxygenase capable of converting chromogenic catechols,
alpha-amylase (Ikuta et al. (1990) Bio/technol. 8:241-242),
tyrosinase (Katz et al. (1983) J Gene Microbiol 129:2703-2714), enzyme which oxidizes tyrosine to give DOPA and dopaquinone which subsequently form melanine, which is readily detectable,
aequorin (Prasher et al. (1985) Biochem Biophys Res Commun 126(3):1259-1268), can be used in the calcium-sensitive bioluminescence detection.

### Target Organisms

Any organism suitable for transformation or delivery of chimeric endonuclease can be used as target organism. This includes prokaryotes, eukaryotes, and archaea, in particular non-human organisms, plants, fungi or yeasts, but also human or animal cells.

In one embodiment the target organism is a plant.

The term “plant” includes whole plants, shoot vegetative organs/structures (e.g. leaves, stems and tubers), roots, flowers and floral organs/structures (e.g. bracts, sepals, petals, stamens, carpels, anthers and ovules), seeds (including embryo, endosperm, and seed coat) and fruits (the mature ovary), plant tissues (e.g. vascular tissue, ground tissue, and the like) and cells (e.g. guard cells, egg cells, trichomes and the like), and progeny of same. The class of plants that can be used in the method of the invention is generally as broad as the class of higher and lower plants amenable to transformation techniques, including angiosperms (monocotyledonous and dicotyledonous plants), gymnosperms, ferns, and multicellular algae. It includes plants of a variety of ploidy levels, including aneuploid, polyploid, diploid, haploid and hemizygous.

Included within the scope of the invention are all genera and species of higher and lower plants of the plant kingdom. Included are furthermore the mature plants, seed, shoots and seedlings, and parts, propagation material (for example seeds and fruit) and cultures, for example cell cultures, derived therefrom.

Preferred are plants and plant materials of the following plant families: Amaranthaceae, Brassicaceae, Carophyllaceae, Chenopodiaceae, Compositae, Cucurbitaceae, Labi-atae, Leguminosae, Papilionoideae, Liliaceae, Linaceae, Malvaceae, Rosaceae, Saxi-fragaceae, Scrophulariaceae, Solanaceae, Tetragoniaceae.

Annual, perennial, monocotyledonous and dicotyledonous plants are preferred host organisms for the generation of transgenic plants. The use of the recombination system, or method according to the invention is furthermore advantageous in all ornamental plants, useful or ornamental trees, flowers, cut flowers, shrubs or turf. Said plant may include—but shall not be limited to—bryophytes such as, for example, Hepaticae (hepaticas) and Musci (mosses); pteridophytes such as ferns, horsetail and club-mosses; gymnosperms such as conifers, cycads, ginkgo and Gnetaeae; algae such as Chlorophyceae, Phaeophpyceae, Rhodophyceae, Myxophyceae, Xanthophyceae, Bacillariophyceae (diatoms) and Euglenophyceae.

Plants for the purposes of the invention may comprise the families of the Rosaceae such as rose, Ericaceae such as rhododendrons and azaleas, Euphorbiaceae such as poinsettias and croton, Caryophyllaceae such as pinks, Solanaceae such as petunias, Gesneriaceae such as African violet, Balsaminaceae such as touch-me-not, Orchida-ceae such as orchids, lridaceae such as gladioli, iris, freesia and crocus, Compositae such as marigold, Geraniaceae such as geraniums, Liliaceae such as drachaena, Moraceae such as ficus, Araceae such as philodendron and many others.

The transgenic plants according to the invention are furthermore selected in particular from among dicotyledonous crop plants such as, for example, from the families of the Leguminosae such as pea, alfalfa and soybean; Solanaceae such as tobacco and many others; the family of the Umbelliferae, particularly the genus Daucus (very particularly the species carota (carrot)) and Apium (very particularly the species graveolens dulce (celery)) and many others; the family of the Solanaceae, particularly the genus Lycopersicon, very particularly the species esculentum (tomato) and the genus Solanum, very particularly the species tuberosum (potato) and melongena (au-bergine) and many others; and the genus Capsicum, very particularly the species annum (pepper) and many others; the family of the Leguminosae, particularly the genus Glycine, very particularly the species max (soybean) and many others; and the family of the Cruciferae, particularly the genus Brassica, very particularly the species napus (oilseed rape), campestris (beet), oleracea cv Tastie (cabbage), oleracea cv Snowball Y (cauliflower) and oleracea cv Emperor (broccoli); and the genus Arabidopsis, very particularly the species thaliana and many others; the family of the Compositae, particularly the genus Lactuca, very particularly the species sativa (lettuce) and many others.

The transgenic plants according to the invention are selected in particular among monocotyledonous crop plants, such as, for example, cereals such as wheat, barley, sorghum and millet, rye, triticale, maize, rice or oats, and sugar cane.

Especially preferred are Arabidopsis thaliana, Nicotiana tabacum, oilseed rape, soybean, corn (maize), wheat, linseed, potato and tagetes.

Plant organisms are furthermore, for the purposes of the invention, other organisms which are capable of photosynthetic activity, such as, for example, algae or cyanobacteria, and also mosses. Preferred algae are green algae, such as, for example, algae of the genus Haematococcus, Phaedactylum tricornatum, Volvox or Dunaliella.

Genetically modified plants according to the invention which can be consumed by humans or animals can also be used as food or feedstuffs, for example directly or following processing known in the art.

### Construction of Polynucleotide Constructs

Typically, polynucleotide constructs (e.g., for an expression cassette) to be introduced into non-human organism or cells, e.g. plants or plant cells are prepared using transgene expression techniques. Recombinant expression techniques involve the construction of recombinant nucleic acids and the expression of genes in transfected cells. Molecular cloning techniques to achieve these ends are known in the art. A wide variety of cloning and in vitro amplification methods suitable for the construction of recombinant nucleic acids are well-known to persons of skill in the art. Examples of these techniques and instructions sufficient to direct persons of skill in the art through many cloning exercises are found in Berger and Kimmel, Guide to Molecular Cloning Techniques, Methods in Enzymology, Vol. 152, Academic Press, hic., San Diego, Calif. (Berger); Current Protocols in Molecular Biology, F. M. Ausubel et al., eds., Current Protocols, a joint venture between Greene Publishing Associates, Inc. and John Wiley & Sons, Inc., (1998 Supplement), T. Maniatis, E. F. Fritsch and J. Sambrook, Molecular Cloning: A Laboratory Manual, Cold Spring Harbor Laboratory, Cold Spring Harbor, N.Y. (1989), in T. J. Silhavy, M. L. Berman and L. W. Enquist, Experiments with Gene Fusions, Cold Spring Harbor Laboratory, Cold Spring Harbor, N.Y. (1984). Preferably, the DNA constructs employed in the invention are generated by joining the abovementioned essential constituents of the DNA construct together in the abovementioned sequence using the recombination and cloning techniques with which the skilled worker is familiar.

The construction of polynucleotide constructs generally requires the use of vectors able to replicate in bacteria. A plethora of kits are commercially available for the purification of plasmids from bacteria. The isolated and purified plasmids can then be further manipulated to produce other plasmids, used to transfect cells or incorporated into Agrobacterium tumefaciens or Agrobacterium rhizogenes to infect and transform plants. Where Agrobacterium is the means of transformation, shuttle vectors are constructed.

Methods for Introducing Constructs into Target Cells

A DNA construct employed in the invention may advantageously be introduced into cells using vectors into which said DNA construct is inserted. Examples of vectors may be plasmids, cosmids, phages, viruses, retroviruses or agrobacteria. In an advantageous embodiment, the expression cassette is introduced by means of plasmid vectors. Preferred vectors are those which enable the stable integration of the expression cassette into the host genome.

A DNA construct can be introduced into the target plant cells and/or organisms by any of the several means known to those of skill in the art, a procedure which is termed transformation (see also Keown et al. (1990) Meth Enzymol 185:527-537). For instance, the DNA constructs can be introduced into cells, either in culture or in the organs of a plant by a variety of conventional techniques. For example, the DNA constructs can be introduced directly to plant cells using ballistic methods, such as DNA particle bombardment, or the DNA construct can be introduced using techniques such as electroporation and microinjection of cells. Particle-mediated transformation techniques (also known as “biolistics”) are described in, e.g., Klein et al. (1987) Nature 327:70-73; Vasil V et al. (1993) Bio/Technol 11:1553-1558; and Becker D et al. (1994) Plant J 5:299-307. These methods involve penetration of cells by small particles with the nucleic acid either within the matrix of small beads or particles, or on the surface. The biolistic PDS-1000 Gene Gun (Biorad, Hercules, Calif.) uses helium pressure to accelerate DNA-coated gold or tungsten microcarriers toward target cells. The process is applicable to a wide range of tissues and cells from organisms, including plants. Other transformation methods are also known to those of skill in the art.

Microinjection techniques are known in the art and are well described in the scientific and patent literature. Also, the cell can be permeabilized chemically, for example using polyethylene glycol, so that the DNA can enter the cell by diffusion. The DNA can also be introduced by protoplast fusion with other DNA-containing units such as minicells, cells, lysosomes or liposomes. The introduction of DNA constructs using polyethylene glycol (PEG) precipitation is described in Paszkowski et al. (1984) EMBO J. 3:2717. Liposome-based gene delivery is e.g., described in WO 93/24640; Mannino and Gould-Fogerite (1988) BioTechniques 6(7):682-691; U.S. Pat. No. 5,279,833; WO 91/06309; and Feigner et al. (1987) Proc Natl Acad Sci USA 84:7413-7414).

Another suitable method of introducing DNA is electroporation, where the cells are permeabilized reversibly by an electrical pulse. Electroporation techniques are described in Fromm et al. (1985) Proc Natl Acad Sci USA 82:5824. PEG-mediated transformation and electroporation of plant protoplasts are also discussed in Lazzeri P (1995) Methods Mol Biol 49:95-106. Preferred general methods which may be mentioned are the calcium-phosphate-mediated transfection, the DEAE-dextran-mediated transfection, the cationic lipid-mediated transfection, electroporation, transduction and infection. Such methods are known to the skilled worker and described, for example, in Davis et al., Basic Methods In Molecular Biology (1986). For a review of gene transfer methods for plant and cell cultures, see, Fisk et al. (1993) Scientia Horticulturae 55:5-36 and Potrykus (1990) CIBA Found Symp 154:198.

Methods are known for introduction and expression of heterologous genes in both monocot and dicot plants. See, e.g., U.S. Pat. No. 5,633,446, U.S. Pat. No. 5,317,096, U.S. Pat. No. 5,689,052, U.S. Pat. No. 5,159,135, and U.S. Pat. No. 5,679,558; Weising et al. (1988) Ann. Rev. Genet. 22: 421-477. Transformation of monocots in particular can use various techniques including electroporation (e.g., Shimamoto et al. (1992) Nature 338:274-276; biolistics (e.g., EP-A1270,356); and Agrobacterium (e.g., Bytebier et al. (1987) Proc Natl Acad Sci USA 84:5345-5349).

In plants, methods for transforming and regenerating plants from plant tissues or plant cells with which the skilled worker is familiar are exploited for transient or stable transformation. Suitable methods are especially protoplast transformation by means of poly-ethylene-glycol-induced DNA uptake, biolistic methods such as the gene gun (“particle bombardment” method), electroporation, the incubation of dry embryos in DNA-containing solution, sonication and microinjection, and the transformation of intact cells or tissues by micro- or macroinjection into tissues or embryos, tissue electroporation, or vacuum infiltration of seeds. In the case of injection or electroporation of DNA into plant cells, the plasmid used does not need to meet any particular requirement. Simple plasmids such as those of the pUC series may be used. If intact plants are to be regenerated from the transformed cells, the presence of an additional selectable marker gene on the plasmid is useful.

In addition to these “direct” transformation techniques, transformation can also be carried out by bacterial infection by means of Agrobacterium tumefaciens or Agrobacterium rhizogenes. These strains contain a plasmid (Ti or Ri plasmid). Part of this plasmid, termed T-DNA (transferred DNA), is transferred to the plant following Agrobacterium infection and integrated into the genome of the plant cell.

For Agrobacterium-mediated transformation of plants, a DNA construct of the invention may be combined with suitable T-DNA flanking regions and introduced into a conventional Agrobacterium tumefaciens host vector. The virulence functions of the A. tumefaciens host will direct the insertion of a transgene and adjacent marker gene(s) (if present) into the plant cell DNA when the cell is infected by the bacteria. Agrobacterium tumefaciens-mediated transformation techniques are well described in the scientific literature. See, for example, Horsch et al. (1984) Science 233:496-498, Fraley et al. (1983) Proc Natl Acad Sci USA 80:4803-4807, Hooykaas (1989) Plant Mol Biol 13:327-336, Horsch RB (1986) Proc Natl Acad Sci USA 83(8):2571-2575), Bevans et al. (1983) Nature 304:184-187, Bechtold et al. (1993) Comptes Rendus De L'Academie Des Sciences Serie III-Sciences De La Vie-Life Sciences 316:1194-1199, Valvekens et al. (1988) Proc Natl Acad Sci USA 85:5536-5540.

A DNA construct of the invention is preferably integrated into specific plasmids, either into a shuttle, or intermediate, vector or into a binary vector). If, for example, a Ti or Ri plasmid is to be used for the transformation, at least the right border, but in most cases the right and the left border, of the Ti or Ri plasmid T-DNA is linked with the expression cassette to be introduced as a flanking region. Binary vectors are preferably used. Bi-nary vectors are capable of replication both in E. coli and in Agrobacterium. As a rule, they contain a selection marker gene and a linker or polylinker flanked by the right or left T-DNA flanking sequence. They can be trans-formed directly into Agrobacterium (Holsters et al. (1978) Mol Gen Genet. 163:181-187). The selection marker gene permits the selection of transformed agrobacteria and is, for example, the nptII gene, which imparts resistance to kanamycin. The Agrobacterium, which acts as host organism in this case, should already contain a plasmid with the vir region. The latter is required for transferring the T-DNA to the plant cell. An Agrobacterium thus transformed can be used for transforming plant cells.

Many strains of Agrobacterium tumefaciens are capable of transferring genetic material—for example a DNA constructs according to the invention—, such as, for example, the strains EHA101 (pEHA101) (Hood E E et al. (1996) J Bacteriol 168(3):1291-1301), EHA105(pEHA105) (Hood et al. 1993, Transgenic Research 2, 208-218), LBA4404(pAL4404) (Hoekema et al. (1983) Nature 303:179-181), C58C1(pMP90) (Koncz and Schell (1986) Mol Gen Genet. 204, 383-396) and C58C1 (pGV2260) (De-blaere et al. (1985) Nucl Acids Res. 13, 4777-4788).

The agrobacterial strain employed for the transformation comprises, in addition to its disarmed Ti plasmid, a binary plasmid with the T-DNA to be transferred, which, as a rule, comprises a gene for the selection of the transformed cells and the gene to be transferred. Both genes must be equipped with transcriptional and translational initiation and termination signals. The binary plasmid can be transferred into the agrobacterial strain for example by electroporation or other transformation methods (Mozo & Hooykaas (1991) Plant Mol Biol 16:917-918). Coculture of the plant explants with the agrobacterial strain is usually performed for two to three days.

A variety of vectors could, or can, be used. In principle, one differentiates between those vectors which can be employed for the Agrobacterium-mediated transformation or agroinfection, i.e. which comprise a DNA construct of the invention within a T-DNA, which indeed permits stable integration of the T-DNA into the plant genome. Moreover, border-sequence-free vectors may be employed, which can be transformed into the plant cells for example by particle bombardment, where they can lead both to transient and to stable expression.

The use of T-DNA for the transformation of plant cells has been studied and described intensively (EP-A1 120 516; Hoekema, In: The Binary Plant Vector System, Offset-drukkerij Kanters B. V., Alblasserdam, Chapter V; Fraley et al. (1985) Crit. Rev Plant Sci 4:1-45 and An et al. (1985) EMBO J. 4:277-287). Various binary vectors are known, some of which are commercially available such as, for example, pBIN19 (Clontech Laboratories, Inc. USA).

To transfer the DNA to the plant cell, plant explants are cocultured with Agrobacterium tumefaciens or Agrobacterium rhizogenes. Starting from infected plant material (for example leaf, root or stalk sections, but also protoplasts or suspensions of plant cells), intact plants can be regenerated using a suitable medium which may contain, for example, antibiotics or biocides for selecting transformed cells. The plants obtained can then be screened for the presence of the DNA introduced, in this case a DNA construct according to the invention. As soon as the DNA has integrated into the host genome, the genotype in question is, as a rule, stable and the insertion in question is also found in the subsequent generations. As a rule, the expression cassette integrated contains a selection marker which confers a resistance to a biocide (for example a herbicide) or an antibiotic such as kanamycin, G 418, bleomycin, hygromycin or phosphinotricin and the like to the transformed plant. The selection marker permits the selection of transformed cells (McCormick et al., Plant Cell Reports 5 (1986), 81-84). The plants obtained can be cultured and hybridized in the customary fashion. Two or more generations should be grown in order to ensure that the genomic integration is stable and hereditary.

The abovementioned methods are described, for example, in B. Jenes et al., Techniques for Gene Transfer, in: Transgenic Plants, Vol. 1, Engineering and Utilization, edited by S D Kung and R Wu, Academic Press (1993), 128-143 and in Potrykus (1991) Annu Rev Plant Physiol Plant Molec Biol 42:205-225). The construct to be expressed is preferably cloned into a vector which is suitable for the transformation of Agrobacterium tumefaciens, for example pBin19 (Bevan et al. (1984) Nucl Acids Res 12:8711).

The DNA construct of the invention can be used to confer desired traits on essentially any plant. One of skill will recognize that after DNA construct is stably incorporated in transgenic plants and confirmed to be operable, it can be introduced into other plants by sexual crossing. Any of a number of standard breeding techniques can be used, depending upon the species to be crossed.

The nucleases or chimeric endonuclease may alternatively be expressed transiently. The chimeric endonuclease may be transiently expressed as a DNA or RNA delivered into the target cell and/or may be delivered as a protein. Delivery as a protein may be achieved with the help of cell penetrating peptides or by fusion with SEciV signal peptides fused to the nucleases or chimeric endonucleases, which mediate the secretion from a delivery organism into a cell of a target organism e.g. from Agrobacterium rhizogenes or Agrobacterium tumefaciens to a plant cell.

### Regeneration of Transgenic Plants

Transformed cells, i.e. those which comprise the DNA integrated into the DNA of the host cell, can be selected from untransformed cells if a selectable marker is part of the DNA introduced. A marker can be, for example, any gene which is capable of conferring a resistance to antibiotics or herbicides (for examples see above). Transformed cells which express such a marker gene are capable of surviving in the presence of concentrations of a suitable antibiotic or herbicide which kill an untransformed wild type. As soon as a transformed plant cell has been generated, an intact plant can be obtained using methods known to the skilled worker. For example, callus cultures are used as starting material. The formation of shoot and root can be induced in this as yet undifferentiated cell biomass in the known fashion. The shoots obtained can be planted and cultured.

Transformed plant cells, derived by any of the above transformation techniques, can be cultured to regenerate a whole plant which possesses the transformed genotype and thus the desired phenotype. Such regeneration techniques rely on manipulation of certain phytohormones in a tissue culture growth medium, typically relying on a biocide and/or herbicide marker that has been introduced together with the desired nucleotide sequences. Plant regeneration from cultured protoplasts is described in Evans et al., Protoplasts Isolation and Culture, Handbook of Plant Cell Culture, pp. 124176, Macmillian Publishing Company, New York (1983); and in Binding, Regeneration of Plants, Plant Protoplasts, pp. 21-73, CRC Press, Boca Raton, (1985). Regeneration can also be obtained from plant callus, explants, somatic embryos (Dandekar et al. (1989) J Tissue Cult Meth 12:145; McGranahan et al. (1990) Plant Cell Rep 8:512), organs, or parts thereof. Such regeneration techniques are described generally in Klee et al. (1987) Ann Rev Plant Physiol 38:467-486.

Combination with Other Recombination Enhancing Techniques

In a further preferred embodiment, the efficacy of the recombination system is increased by combination with systems which promote homologous recombination. Such systems are described and encompass, for example, the expression of proteins such as RecA or the treatment with PARP inhibitors. It has been demonstrated that the intrachromosomal homologous recombination in tobacco plants can be increased by using PARP inhibitors (Puchta H et al. (1995) Plant J. 7:203-210). Using these inhibitors, the homologous recombination rate in the recombination cassette after induction of the sequence-specific DNA double-strand break, and thus the efficacy of the deletion of the transgene sequences, can be increased further. Various PARP inhibitors may be employed for this purpose. Preferably encompassed are inhibitors such as 3-aminobenzamide, 8-hydroxy-2-methylquinazolin-4-one (NU1025), 1,11b-dihydro-(2H)benzopyrano(4,3,2-de)isoquinolin-3-one (GPI 6150), 5-aminoisoquino-linone, 3,4-dihydro-5-(4-(1-piperidinyl)butoxy)-1(2H)-isoquinolinone, or the compounds described in WO 00/26192, WO 00/29384, WO 00/32579, WO 00/64878, WO 00/68206, WO 00/67734, WO 01/23386 and WO 01/23390.

In addition, it was possible to increase the frequency of various homologous recombination reactions in plants by expressing the E. coli RecA gene (Reiss B et al. (1996) Proc Natl Acad Sci USA 93(7):3094-3098). Also, the presence of the protein shifts the ratio between homologous and illegitimate DSB repair in favor of homologous repair (Reiss B et al. (2000) Proc Natl Acad Sci USA 97(7):3358-3363). Reference may also be made to the methods described in WO 97/08331 for increasing the homologous recombination in plants. A further increase in the efficacy of the recombination system might be achieved by the simultaneous expression of the RecA gene or other genes which increase the homologous recombination efficacy (Shalev G et al. (1999) Proc Natl Acad Sci USA 96(13):7398-402). The above-stated systems for promoting homologous recombination can also be advantageously employed in cases where the recombination construct is to be introduced in a site-directed fashion into the genome of a eukaryotic organism by means of homologous recombination.

### Methods of Providing Chimeric Endonucleases:

The current invention provides a method of providing a chimeric endonuclease as described above.

The method comprises the steps of:
a. providing at least one endonuclease coding region
b. providing at least one heterologous DNA binding domain coding region,
c. providing a polynucleotide having a potential DNA recognition sequence or potential DNA recognition sequences of the endonuclease or endonucleases of step a) and having a potential recognition sequence or having potential recognition sequences of the heterologous DNA binding domain or heterologous DNA binding domains of step b),
d. creating a translational fusion of all endonuclease coding regions of step b) and all heterologous DNA binding domains of step c),
e. expressing a chimeric endonuclease from the translational fusion created in step d),
f. testing the chimeric endonuclease expressed in step e) for cleavage of the polynucleotide of step c).

Depending on the intended purpose, the method steps a), b), c) and d) can be used in varying order. For example, the method can be used to provide a particular combination of at least one endonuclease and at least one heterologous DNA binding domain and providing thereafter a polynucleotide comprising potential DNA recognition sites and potential recognition sites reflecting the order in which the at least one nuclease and the at least one heterologous DNA binding site were arranged in the translational fusion, and testing the chimeric endonuclease for cleaving activity on a polynucleotide having potential DNA recognition sites and potential recognition sites for the nucleases and heterologous DNA binding domains comprised by the chimeric endonuclease and selecting at least one polynucleotide that is cut by the chimeric endonuclease.

The method can also be used to design a chimeric endonuclease for cleaving activity on a preselected polynucleotide, by first providing a polynucleotide having a specific sequence, thereafter selecting at least one endonuclease and at least one heterologous DNA binding domain having non-overlapping potential DNA recognition sites and potential recognition sites in the nucleotide sequence of the polynucleotide, creating a translational fusion of the at least one endonuclease and the at least one heterologous DNA binding domain, expressing the chimeric endonuclease encoded by said translational fusion and testing the chimeric endonuclease of cleavage activity on the preselected polynucleotide sequence, and selecting a chimeric endonuclease having such cleavage activity.

This method can be used to design a chimeric endonuclease having an enhanced cleavage activity on a specific polynucleotide, for example, if a polynucleotide comprises a DNA recognition site of a nuclease it will be possible to identify a potential recognition site of a heterologous DNA binding domain, which can be used to create a chimeric endonuclease comprising the nuclease and the heterologous DNA binding domain.

Alternatively, this method can also be used to create a chimeric endonuclease having cleavage activity on a specific polynucleotide comprising a recognition site of a heterologous DNA binding domain. For example, in case the specific polynucleotide is known to be bound by a heterologous DNA binding domain, e.g. a particular transcription factor or a virulence factor of a pathogen having a specific DNA binding activity, like Tal-Type Effector proteins or there repeat units in particular Tal-Type III Effector proteins of Xanthomonas species, it is possible to identify a endonuclease having a potential DNA recognition site close to but not overlapping with the recognition site of the identified heterologous DNA binding domain. By creating a translational fusion and expressing the chimeric endonuclease comprising the identified endonuclease and the heterologous DNA binding domain, it will be possible to test the chimeric endonuclease for cleavage activity on said preselected polynucleotide.

Suitable endonucleases and heterologous DNA binding domains can be identified by searching databases comprising DNA recognition sites of endonucleases and recognition sites of DNA binding proteins like transcription factors or virulence factors.

Further, it is possible to mutate the amino acid sequence of endonucleases, like I-SceI, I-CreI, I-DmoI or I-MsoI to create new binding and DNA cleavage activity. Similar techniques are available to create new binding activities of zink-finger comprising proteins or virulence factors of the Tal-Type III Effector proteins of Xanthomonas species, which can be used as heterologous DNA binding domains. By creating chimeric endonucleases comprising endonucleases like I-SceI, I-CreI, I-DmoI or I-MsoI and heterologous DNA binding domains derived from or comprising zink-finger proteins or Tal-Type III Effector proteins of Xanthomonas species in combination with mutational techniques to adapt their DNA binding activity to the sequence of preselected polypeptides, it is possible to create chimeric endonucleases which will bind and cleave such preselected polypeptides.

Accordingly one embodiment of the invention comprises chimeric endonucleases comprising
a) at least one endonuclease selected from the group of I-SceI, I-CreI, I-DmoI or I-MsoI or homologs of I-SceI, I-CreI, I-DmoI or I-MsoI having at least 80%, 85%, 90% 95%, 96%, 97%, 98% or 99% sequence identity, and
b) a heterologous DNA binding domain comprising either at least one zink finger protein or comprising at least one Tal-Type III Effector protein of Xanthomonas species or comprising at least one zink finger protein and comprising at least one Tal-Type III Effector protein of Xanthomonas species or comprising at least one homolog of zink finger proteins or Tal-Type III Effector proteins of Xanthomonas species having at least 80%, 85%, 90% 95%, 96%, 97%, 98% or 99% sequence identity.

The cleavage activity of endonucleases and chimeric endonucleases as well as the DNA binding activity of endonucleases, heterologous DNA binding domains and chimeric endonucleases can be tested by in vitro and in vivo techniques known in the art. For example by techniques as disclosed in the examples herein.

### Methods for Homologous Recombination and Targeted Mutation Using Chimeric Endonucleases.

The current invention provides a method for homologous recombination of polynucleotides comprising:
a. providing a cell competent for homologous recombination,
b. providing a polynucleotide comprising a recombinant polynucleotide flanked by a sequence A and a sequence B,
c. providing a polynucleotide comprising sequences A′ and B′, which are sufficiently long and homologous to sequence A and sequence B, to allow for homologous recombination in said cell and
d. providing a chimeric endonuclease or an expression cassette coding for a chimeric endonuclease,
e. combining b), c) and d) in said cell and
f. detecting recombined polynucleotides of b) and c), or selecting for or growing cells comprising recombined polynucleotides of b) and c).

In one embodiment of the invention, the polynucleotide provided in step b) comprises at least one chimeric recognition site, preferably a chimeric recognition site selected from the group of sequences described by SEQ ID NO: 14, 15, 16, 17, 18, 19 or 20.

In one embodiment of the invention, the polynucleotide provided in step c) comprises at least one chimeric recognition site, preferably selected from the group of sequences described by SEQ ID NO: SEQ ID NO: 14, 15, 16, 17, 18, 19 or 20.

In one embodiment of the invention, the polynucleotide provided in step b) and the polynucleotide provided in step c) comprise at least one chimeric recognition site, preferably selected from the group of sequences described by SEQ ID NO: 14, 15, 16, 17, 18, 19 or 20.

In one embodiment of the invention, step e) leads to deletion of a polynucleotide comprised in the polynucleotide provided in step c).

In one embodiment of the invention the deleted polynucleotide comprised in the polynucleotide provided in step c) codes for a marker gene or parts of a marker gene.

In one embodiment of the invention, the polynucleotide provided in step b) comprises at least one expression cassette.

In one embodiment of the invention, the polynucleotide provided in step b) comprises at least one expression cassette. leading to expression of a selection marker gene or a reporter gene.

In one embodiment of the invention, the polynucleotide provided in step b) comprises at least one expression cassette. leading to expression of a selection marker gene or a reporter gene and comprises at least one DNA recognition site or at least one chimeric recognition site.

A further embodiment of the invention provides a method for targeted mutation of polynucleotides comprising:
a. providing a cell comprising a polynucleotide comprising a chimeric recognition site,
b. providing a chimeric endonuclease, e.g. an chimeric endonuclease comprising an endonuclease having a sequence selected from the group of sequences described by SEQ ID NO: 2, 3, or 5, and being able to cleave the chimeric recognition site of step a),
c. combining a) and b) in said cell and
d. detecting mutated polynucleotides, or selecting for growing cells comprising mutated polynucleotides.

The invention provides in another embodiment a method for homologous recombination as described above or a method for targeted mutation of polynucleotides as described above, comprising:

combining the chimeric endonuclease and the chimeric recognition site via crossing of organisms, via transformation of cells or via a SecIV peptide fused to the chimeric endonuclease and contacting the cell comprising the chimeric recognition site with an organism expressing the chimeric endonuclease and expressing a SecIV transport complex able to recognize the SecIV peptide fused to the chimeric endonuclease.

### EXAMPLES

### General Methods

The chemical synthesis of oligonucleotides can be effected for example in the known manner using the phosphoamidite method (Voet, Voet, 2nd edition, Wiley Press New York, pages 896-897). The cloning steps carried out for the purposes of the present invention, such as, for example, restriction cleavages, agarose gel electrophoresis, purification of DNA fragments, the transfer of nucleic acids to nitrocellulose and nylon membranes, the linkage of DNA fragments, the transformation of E. coli cells, bacterial cultures, the propagation of phages and the sequence analysis of recombinant DNA are carried out as described by Sambrook et al. (1989) Cold Spring Harbor Laboratory Press; ISBN 0-87969-309-6. Recombinant DNA molecules were sequenced using an ALF Express laser fluorescence DNA sequencer (Pharmacia, Upsala [sic], Sweden) following the method of Sanger (Sanger et al., Proc. Natl. Acad. Sci. USA 74 (1977), 5463-5467).

### Example 1

### Constructs Harboring Sequence Specific DNA-Endonuclease Expression Cassettes for Expression in E. coli

### Example 1a

### Basic Construct

In this example we present the general outline of a vector, named “Construct I” suitable for transformation in E. coli. This general outline of the vector comprises an ampicillin resistance gene for selection, a replication origin for E. coli and the gene araC, which encodes an Arabinose inducible transcription regulator. Different genes, encoding the different versions of the sequence specific DNA-endonuclease, can be expressed from the Arabinose inducible pBAD promoter (Guzman et al., J Bacterial 177: 4121-4130 (1995)). The sequences of the genes encoding the different nuclease versions are given in the following examples.

The control construct, in which encodes the sequence of I-SceI (SEQ ID NO: 22), was called VC-SAH40-4.

### Example 1b

### scTet-I-SceI Fusion Constructs

In JOURNAL OF BACTERIOLOGY 150(2), 633-642 (1982) Beck et al. described the TetR protein. TetR acts as a dimer, but single chain variants (scTetR) are well described in NUCLEIC ACIDS RESEARCH 31(12), 3050-3056 (2003) by Krueger et al. The scTetR encoding sequence was fused to I-SceI, with a single lysine as a short. The linker was designed in a way that the resulting fusion protein recognizes a cognate binding site, which represents a combination of the binding sites of I-SceI and TetR. TetR is a transcriptional repressor, which binds to the DNA in absence of the inducer. It is displaced from the recognition sequence in the presence of tetracycline. This could provide the potential to regulate the activity or DNA binding affinity of the fusion protein in the same manner. The resulting plasmid was called VC-SAH54-4. The sequence of the construct is identical to the sequence of construct I, whereas the nuclease encoding gene was replaced by the sequence described by SEQ ID NO: 23.

A similar construct was generated, which in addition to the latter contains a NLS sequence. The resulting plasmid was called VC-SAH53-10. The sequence of the construct is identical to the sequence of construct I, whereas the nuclease encoding gene was replaced by the sequence described by SEQ ID NO: 24.

### Example 1c

### scArc-I-SceI Fusion Constructs

In J Mol Biol 185 (2), 445-6 (1985) Jordan et al. described the crystallization of the Arc Repressor of Salmonella phage P22 Arc. It is active as a dimer, but single chain variants (scArc) are described in Biochemistry 35 (1), 109-16 (1996) by Robinsons et al. The coding sequence for this single chain variant was fused to I-SceI, with a linker that encompasses a NLS. The linker having the amino acid sequence: RSGGGSGGGTGGGSGGGAPKKKRKVLE (SEQ ID NO: 151) was designed in a way that the resulting fusion protein recognizes a cognate binding site, which represents a combination of the binding sites of I-SceI and Arc. The resulting plasmid was called VC-SAH28-5. The sequence of the construct is identical to the sequence of construct I, whereas the encoded gene is described by SEQ ID NO: 25. Also a fusion with a shorter linker the linker having the amino acid sequence: RSAPKKKRKVLE (SEQ ID NO: 152) between scArc and I-SceI was generated, which still encompasses a NLS. The resulting plasmid was called VC-SAH46-4. The sequence of the construct is identical to the sequence of Construct I, whereas the encoded gene is described by SEQ ID NO: 26.

### Example 2

### Constructs Harboring Nuclease Recognition Sequences/Target Sites to Monitor I-SceI Activity in E. coli

### Example 2a

### Basic Construct

In this example we present the general outline of a vector, named “Construct II” suitable for transformation in E. coli. This general outline of the vector comprises a Kanamycin resistance gene for selection, a replication origin for E. coli, which is compatible with the on of Construct I. SEQ ID NO: 27 shows a sequence stretch of “NNNNNNNNNN”. This is meant to be a placeholder for different recognition/target sites for the diverse versions and protein fusions of the sequence specific DNA-endonucleases. The control construct, in which the placeholder is replaced by a sequence stretch encompassing the native target sequence of I-SceI (SEQ ID NO: 28), was called VC-SAH6-1. A control plasmid without a target site was called VC-SAH7-1 (SEQ ID NO 29)

The different combined target sites are given in the following examples.

### Example 2b

target sites combined of I-SceI recognition sequence and scTet binding sequence Combined target sites were generated, that consist of the target site of the nuclease I-SceI and TetR. Different combined target sites with varying distances of the single sites were generated. The goal was to identify the one that is best recognized by the cognate I-SceI fusion protein. The resulting plasmids were called VC-SAH60-5, VC-SAH61-1, VC-SAH62-1. The sequence of the constructs is identical to the sequence of Construct II, whereas the sequence “NNNNNNNNNN” was replaced by the sequences described by SEQ ID NO: 30, NO: 31, NO: 32, respectively.

### Example 2c

target sites combined of I-SceI recognition sequence and scArc binding sequence In PNAS 96, 811-817 (1999) Schildbach et al. described the Arc Protein in contact with its cognate recognition sequence. Combined target sites were generated, that consist of the target site of the nuclease I-SceI and Arc, with varying distances. The goal is to identify the one that is best recognized by the cognate I-SceI fusion protein. The resulting plasmids are called VC-SAH132-1, VC-SAH133-8, VC-SAH134-1 and VC-SAH135-1. The sequences of these plasmids is identical to the sequence of Construct III (SEQ ID NO: 33), where the sequence “NNNNNNNNNN” is replaced by the sequences consisting of different versions of the combined target sites, described by SEQ ID NO: 34, NO: 35, NO: 36, NO: 37 respectively.

### Example 3

### Cotransformation of DNA Endonuclease Encoding Constructs and Constructs Harboring Nuclease Recognition Sequences

Two plasmids with different selection markers and identical concentrations were transformed in chemical competent E. coli Top10 cells, according to the manufacturer description. The cells were plated on LB with the respective antibiotics for selection, and grown over night at 37° C. With this method constructs harboring sequence specific DNA-endonuclease expression cassettes and cognate constructs harboring nuclease recognition sequences/target sites were combined in the same transformant to allow monitoring of the nuclease activity.

### Example 4

### Demonstration of the Endonuclease Activity in E. Coli

Cotransformants which carry the combination of two plasmids, one encoding a nuclease or a nuclease-fusion (Construct I) and the other one harboring a compatible target site (Construct II) were grown over night in LB with Ampicillin and Kanamycin. The cultures were diluted 1:100 and grown until they reached OD₆₀₀=0.5. The expression of the fusion protein from Construct I was induced by addition of Arabinose for 3 to 4 hours. The pBAD promoter is described to be dose dependent (Guzman 1995), therefore the culture was divided in different aliquots and protein expression was induced with Arabinose concentrations varying from 0.2% to 0.0002%. 5 μl of each aliquot were plated on LB solid media, supplemented with Ampicillin and Kanamycin. The plates were incubated over night at 37° C. and cell growth was analyzed semi quantitatively. Active nuclease fusions did cut the constructs, which harbor the target site. This led to the loss of Construct II or Construct III, which confer Kanamycin resistance. Therefore, activity of the fusion protein was observed due to the lost ability of the cotransformants to grow on Kanamycin containing medium.

### Results:

The result are simplified and summarized in Table 9. ++ and + represent very strong and strong growth, which indicates no or little activity of the expressed nuclease towards the respective target site.—and—represent reduced or no growth, which indicates high or very high activity of the nuclease towards the respective target site.

TABLE 9
I-SceI-scTet fusions: E. coli growth assay indicates
endonuclease activity (enzymatic acitivity) against the
respective target sites.

| | VC-SAH40-4 | VC-SAH54-4 | VC-SAH53-10 |
|---|---|---|---|
| VC-SAH7-1 | ++ | ++ | ++ |
| VC-SAH6-1 | −− | −− | −− |
| VC-SAH60-5 | | −− | −− |
| VC-SAH61-1 | | −− | −− |
| VC-SAH62-1 | | −− | −− |

### Example 5

### Transformation of Arabidopsis thaliana

A. thaliana plants were grown in soil until they flowered. Agrobacterium tumefaciens (strain C58C1 [pMP90]) transformed with the construct of interest was grown in 500 mL in liquid YEB medium (5 g/L Beef extract, 1 g/L Yeast Extract (Duchefa), 5 g/L Peptone (Duchefa), 5 g/L sucrose (Duchefa), 0.49 g/L MgSO₄ (Merck)) until the culture reached an OD₆₀₀ 0.8-1.0. The bacterial cells were harvested by centrifugation (15 minutes, 5,000 rpm) and resuspended in 500 mL infiltration solution (5% sucrose, 0.05% SILWET L-77 [distributed by Lehle seeds, Cat. No. VIS-02]). Flowering plants were dipped for 10-20 seconds into the Agrobacterium solution. Afterwards the plants were kept in the dark for one day and then in the greenhouse until seeds could be harvested. Transgenic seeds were selected by plating surface sterilized seeds on growth medium A (4.4 g/L MS salts [Sigma-Aldrich], 0.5 g/L MES [Duchefa]; 8 g/L Plant Agar [Duchefa]) supplemented with 50 mg/L kanamycin for plants carrying the nptII resistance marker gene, and 10 mg/L Phosphinotricin for plants carrying the pat gene, respectively. Surviving plants were transferred to soil and grown in the greenhouse.

### Example 6

### Constructs Harbouring Sequence Specific DNA-Endonuclease Expression Cassettes for A. thaliana

### Example 6a

### Basic Construct

In this example we present the general outline of a binary vector, named “Construct IV” suitable for plant transformation. This general outline of the binary vector comprises a T-DNA with a p-Mas1del100::cBAR::t-Ocs1 cassette, which enables selection on Phosphinotricin, when integrated into the plant genome. SEQ ID NO: 38 shows a sequence stretch of “NNNNNNNNNN”. This is meant to be a placeholder for genes encoding the different versions of the sequence specific DNA-endonuclease. The sequence of the latter is given in the following examples.

### Example 6b

### scTet-I-SceI Fusion Constructs

The sequence stretch of “NNNNNNNNNN” of construct IV is separately replaced by genes encoding the different versions of I-SceI-scTet fusions. The scTetR encoding sequence was fused to I-SceI, with a short linker, as described in Example 1c). The resulting plasmid is called VC-SAH140. The sequence of the construct is identical to the sequence of construct IV, whereas the sequence “NNNNNNNNNN” is replaced by the sequence described in Example 1.

A similar construct is generated, which in addition to the latter contains a NLS sequence. The resulting plasmid is called VC-SAH139-20. The sequence of the construct is identical to the sequence of construct I, whereas the sequence “NNNNNNNNNN” is replaced by the sequence described in Example 1.

### Example 6c

### scArc-I-SceI Fusion Constructs

The sequence stretch of “NNNNNNNNNN” of construct IV was separately replaced by genes encoding the different versions of I-SceI-scArc fusions. The scArc encoding sequence was fused to I-SceI, as described in Example 1d). The resulting plasmid was called VC-SAH89-10. The sequence of the construct is identical to the sequence of construct IV, whereas the sequence “NNNNNNNNNN” was replaced by the sequence described in Example 1d). Another fusion with a shorter linker between scArc and I-SceI is generated, which still encompasses a NLS. The resulting plasmid is called VC-SAH90. The sequence of the construct is identical to the sequence of construct IV, whereas the sequence “NNNNNNNNNN” is replaced by the sequence described by SEQ ID NO: 26.

### Example 7

### Constructs Harboring Nuclease Recognition Sequences/Target Sites to Monitor Nuclease Activity in A. thaliana

### Example 7a Basic Construct

In this example we present the general outline of a binary vector, named “Construct V”, suitable for transformation in A. thaliana. This general outline of the vector comprises a T-DNA with a nos-promoter::nptII::nos-terminator cassette, which confers kanamycin resistance when integrated into the plant genome.

The T-DNA also comprises a partial uidA (GUS) gene (called “GU”) and another partial uidA gene (called “US”). Between GU and US a stretch of “NNNNNNNNNN” is shown in SEQ ID NO: 39. This is meant to be a placeholder for different recognition/target sites for the diverse versions and protein fusions of the sequence specific DNA-endonucleases. The sequences of the different target sites are given in the following examples.

If the recognition sequence is cut by the respective nuclease, the partially overlapping and non-functional halves of the GUS gene (GU and US) will be restored as a result of intrachromosomal homologous recombination (ICHR). This can be monitored by histochemical GUS staining (Jefferson 1985).

### Example 7b

### Target Sites Combined of Nuclease Recognition Sequence and scTet Binding Sequence

Combined target sites are generated, that consist of the target site of the nuclease I-SceI and TetR. Different combined target sites with varying distances of the single sites are generated. The goal is to identify the one that is best recognized by the cognate I-SceI fusion protein. The resulting plasmids are called VC-SAH113, VC-SAH114, VC-SAH115. The sequence of the constructs is identical to the sequence of Construct II, whereas the sequence “NNNNNNNNNN” is replaced by the sequences described by SEQ ID NO: 40, NO: 41, NO: 42, respectively.

### Example 7c

### Target Sites Combined of Nuclease Recognition Sequence and scArc Binding Sequence

Combined target sites were generated, that consist of the target site of the nuclease I-SceI and Arc. Different combined target sites with varying distances of the single sites were generated. The goal was to identify the one that is best recognized by the cognate I-SceI fusion protein. The resulting plasmids were called VC-SAH16-4, VC-SAH17-8, VC-SAH18-7, VC-SAH19-15. The sequence of the constructs is identical to the sequence of Construct V, whereas the sequence “NNNNNNNNNN” was replaced by the sequences described by SEQ ID NO: 43, NO: 44, NO: 45, NO: 46 respectively.

### Example 8

### Transformation of Sequence-Specific DNA Endonuclease Encoding Constructs into A. thaliana

Plasmids VC-SAH87-4 VC-SAH140, VC-SAH139-20, VC-SAH89-10, VC-SAH90 were/are transformed into A. thaliana according to the protocol described in Example 5. Selected trans-genic lines (T1 generation) are grown in the greenhouse and some flowers will be used for crossings (see below).

### Example 9

### Transformation of Constructs Harboring Combined Target Sites to Monitor Recombination Into A. thaliana

Plasmids VC-SAH111, VC-SAH112, VC-SAH113, VC-SAH114, VC-SAH115, VC-SAH16-4, VC-SAH17-8, VC-SAH18-7 and VC-SAH19-15 were/are transformed into A. thaliana according to the protocol described in Example 5. Selected transgenic lines (T1 generation) are grown in the greenhouse and some flowers are used for crossings (see Example 10).

### Example 10

### Monitoring Activity of the Nuclease Fusions in A. thaliana

Transgenic lines of Arabidopsis harboring a T-DNA encoding a sequence-specific DNA endonuclease are crossed with lines of Arabidopsis harboring the T-DNA carrying a GU-US reporter construct with a corresponding combined target site. As a result of I-SceI activity on the target site a functional GUS gene will be restored by homologous intrachromosomal recombination (ICHR). This can be monitored by histochemical GUS staining (Jefferson et al. (1987) EMBO J 6:3901-3907).

To visualize I-SceI activity of the scTet fusions, transgenic lines of Arabidopsis harboring the T-DNA of the nuclease encoding constructs VC-SAH139-20 and VC-SAH140 are crossed with lines of Arabidopsis harboring the T-DNA of constructs VC-SAH113, VC-SAH114, VC-SAH115, harboring the target sites.

To visualize I-SceI activity of the scArc fusions, transgenic lines of Arabidopsis harboring the T-DNA of the nuclease encoding constructs VC-SAH89-10, VC-SAH90 are crossed with lines of A. thaliana harboring the T-DNA of constructs VC-SAH16-4, VC-SAH17-8, VC-SAH18-7, VC-SAH19-15, harboring the target sites.

F1 seeds of the crosses are harvested. The seeds are surface sterilized and grown on medium A supplemented with the respective antibiotics and/or herbicides. Leafs are harvested and used for histochemical GUS staining. The percentage of plants showing blue staining is an indicator of the frequency of ICHR and therefore for I-SceI activity.

Activity of the different fusion proteins is determined by comparison of the number ICHR events of these crossings. An increase in specificity of the I-SceI fusions with respect to the native nuclease will be observed by comparing these results with control crosses. For these all trans-genic lines of Arabidopsis harboring the T-DNA of constructs encoding the different fusions of 1-SceI are crossed with lines of Arabidopsis harboring the T-DNA of the construct carrying the native I-SceI target site (VC-SAH743-4).

The next generation of these plants is analyzed for fully blue seedlings.

1. A chimeric endonuclease comprising at least one endonuclease having DNA double strand break inducing activity and at least one heterologous DNA binding domain.

2. The chimeric endonuclease of claim 1, comprising at least I-SceI, I-CreI, I-CeuI, I-ChuI, I-DmoI, Pi-SceI, I-MsoI, or I-AniI, or a LAGLIDADG endonuclease having at least 45% amino acid sequence identity to any one of I-SceI, I-CreI, I-CeuI, I-ChuI, I-DmoI, Pi-SceI, I-MsoI, or I-AniI.

3. The chimeric endonuclease of claim 2, wherein the LAGLIDADG endonuclease has at least 80% amino acid sequence identity to a polypeptide described by SEQ ID NO: 1, 2, 3 or 159.

4. The chimeric endonuclease of claim 1, comprising a heterologous DNA binding domain derived from a transcription factor or an inactive nuclease, or a fragment comprising a DNA binding domain of a transcription factor or a nuclease.

5. The chimeric endonuclease of claim 1, wherein at least one heterologous DNA binding domain is an inactive I-SceI, I-CreI, I-CeuI, I-ChuI, I-DmoI, PI-SceI, I-MsoI, or I-AniI, or an inactive homolog thereof having at least 45% amino acid sequence identity to I-SceI, I-CreI, I-CeuI, I-ChuI, I-DmoI, PI-SceI, I-MsoI, or I-AniI.

6. The chimeric endonuclease of claim 1, wherein the chimeric endonuclease comprises an engineered endonuclease or an optimized endonuclease or an engineered optimized endonuclease.

7. The chimeric endonuclease of claim 1, wherein at least one heterologous DNA binding domain is a transcription factor or a DNA binding domain of a transcription factor comprising a HTH domain.

8. The chimeric endonuclease of claim 1, wherein at least one transcription factor or the DNA binding domain of a transcription factor comprises a HTH domain comprising an amino acid sequence of at least 80% sequence identity to at least one amino acid sequence described by SEQ ID NO: 91, 92, 93, 94, 95, 96, 97, 98, 99, 100, 101, 102, 103, 104, 105, 106, 107, 108, 109, 110, 111, 112, 113, 114, 115, 116, 117, 118 or 119, preferably described by 91, 92, 93, 94, 95, 112, 113, 114, 115, 116, 117, 118 or 119.

9. The chimeric endonuclease of claim 1, wherein the heterologous DNA binding domain comprises a polypeptide having at least 80% amino acid sequence identity to a polypeptide described by SEQ ID NO: 6 or 7.

10. The chimeric endonuclease of claim 1, wherein the endonuclease having DNA double strand break inducing activity and the heterologous DNA binding domain are connected via a linker polypeptide.

11. The chimeric endonuclease of claim 1, wherein the DNA binding activity of the heterologous DNA binding domain is inducible.

12. The chimeric endonuclease of claim 1, wherein the DNA binding activity of the heterologous DNA binding domain is inducible by at least one mechanism selected from the group consisting of:
a) binding of an inducer molecule,
b) binding of the second monomer of the DNA binding domain,
c) phosphorylation or dephosphorylation, and
d) a rising of temperature or a lowering of temperature.

13. The chimeric endonuclease of claim 1, wherein the DNA double strand break inducing activity of the endonuclease is inducible by expression of the second monomer of a homo- or heterodimeric endonuclease.

14. The chimeric endonuclease of claim 1, comprising at least one NLS-sequence or one or more SecIII or SecIV secretion signals, or a combination of one or more NLS-sequences and one or more SecIII or SecIV secretion signals, or a combination of one or more SecIII and SecIV secretion signals with one or more NLS-sequences.

15. An isolated polynucleotide comprising a nucleotide sequence coding for the chimeric endonuclease of claim 1.

16. The isolated polynucleotide of claim 15, wherein the nucleotide sequence
a) is codon optimized,
b) has a low content of RNA instability motifs,
c) has a low content of codon repeats,
d) has a low content of cryptic splice sites,
e) has a low content of alternative start codons,
f) has a low content of restriction sites,
g) has a low content of RNA secondary structures, or
h) has any combination of a), b), c), d), e), f) or g).

17. An expression cassette comprising the isolated polynucleotide of claim 15 in functional combination with a promoter and a terminator sequence.

18. An isolated polynucleotide comprising a chimeric recognition sequence having a length of about 15 to about 300 nucleotides and comprising:
a) a recognition sequence of an endonuclease, and
b) a recognition sequence of a heterologous DNA binding domain.

19. The isolated polynucleotide of claim 18, wherein the recognition sequence of an endonuclease is a recognition sequence of a LAGLIDADG endonuclease.

20. The isolated polynucleotide of claim 18, comprising:
a) a DNA recognition sequence of I-SceI,
b) a recognition sequence of scTet or scArc, and
c) a linker sequence of 0 to 10 nucleotides connecting the DNA recognition sequence of I-SceI and the recognition sequence of scTet or scArc.

21. The isolated polynucleotide of claim 18, comprising a polynucleotide sequence as described by any one of SEQ ID NO: 14, 15, 16, 17, 18, 19 or 20.

22. A vector, a host cell, or a nonhuman organism comprising:
a) a polynucleotide coding for the chimeric endonuclease of claim 1,
b) an expression cassette comprising the polynucleotide of a) in functional combination with a promoter and a terminator sequence,
c) an isolated polynucleotide comprising a chimeric recognition sequence having a length of about 15 to about 300 nucleotides and comprising a recognition sequence of an endonuclease and a recognition sequence of a heterologous DNA binding domain, or
d) any combination of a), b) and c).

23. The non-human organism of claim 22, wherein the non-human organism is a plant.

24. A method for providing a chimeric endonuclease, comprising:
a) providing at least one endonuclease coding region,
b) providing at least one heterologous DNA binding domain coding region,
c) providing a polynucleotide having a potential DNA recognition sequence or potential DNA recognition sequences of the endonuclease or endonucleases of step a) and having a potential recognition sequence or having potential recognition sequences of the heterologous DNA binding domain or heterologous DNA binding domains of step b),
d) creating a translational fusion of the coding regions of all endonucleases of step b) and all heterologous DNA binding domains of step c),
e) expressing a chimeric endonuclease from the translational fusion created in step d), and
f) testing the chimeric endonuclease expressed in step e) for cleavage of the polynucleotide of step c).

25. A method for homologous recombination of polynucleotides comprising:
a) providing a cell competent for homologous recombination,
b) providing a polynucleotide comprising the isolated polynucleotide of claim 18 flanked by a sequence A and a sequence B,
c) providing a polynucleotide comprising a sequence A′ and a sequence B′, which are sufficiently long and homologous to the sequence A and the sequence B, to allow for homologous recombination in said cell,
d) providing a chimeric endonuclease comprising at least one endonuclease having DNA double strand break inducing activity and at least one heterologous DNA binding domain, or an expression cassette comprising a nucleotide sequence encoding said chimeric endonuclease in functional combination with a promoter and a terminator sequence,
e) combining the polynucleotides of b), c) and the chimeric endonuclease of d) in said cell, and
f) detecting recombined polynucleotides of b) and c), or selecting for and/or growing cells comprising recombined polynucleotides of b) and c).

26. The method of claim 25, wherein upon homologous recombination a polynucleotide sequence comprised in the competent cell of step a) is deleted from the genome of the growing cells of step f).

27. A method for targeted mutation of polynucleotides comprising:
a) providing a cell comprising a polynucleotide comprising a chimeric recognition site or a DNA recognition site,
b) providing a chimeric endonuclease of claim 1, or an expression cassette comprising a nucleotide sequence encoding said chimeric endonuclease in functional combination with a promoter and a terminator sequence, and being able to cleave the chimeric recognition site or the DNA recognition site of step a),
c) combining the polynucleotide of a) and the chimeric endonuclease of b) in said cell, and
d) detecting mutated polynucleotides, or selecting for or growing cells comprising mutated polynucleotides.

28. The method of claim 25, wherein the chimeric endonuclease and the chimeric recognition site are combined in at least one cell via crossing of organisms, via transformation, or via transport mediated via a SecIII or SecIV peptide fused to the chimeric endonuclease.
